SEQUENCE LISTING (87 sequences)

SEQ ID NO: 1 - RNA, 46 nt, Artificial Sequence
  t-NAT21
uuaauacacccacuucauggauaaguaaaacugaggcuuggagaag

SEQ ID NO: 2 - RNA, 62 nt, Artificial Sequence
  t-NAT1
uuaauacacccacuucauggauaaguaaaacugaggcuuggagaagcccacauugacacagc

SEQ ID NO: 3 - RNA, 63 nt, Artificial Sequence
  t-NAT2s
uuaauacacccacuucauggauaaguaaaacugaggcuuggagaagcccacauugacacagca

SEQ ID NO: 4 - DNA, 74 bp, Artificial Sequence
  MIR3 reverse-complement
  misc_feature (8)..(8): TBC
  misc_feature (8)..(8): n=a,c,g or t
ccaacccnctcattttacagatgaggaaaactgaggcccagagaggtgaagtgacttgcccaaggtcacacagc

SEQ ID NO: 5 - DNA, 73 bp, Artificial Sequence
  MIR reverse-complement
ttattatccccattttacagatgaggaaactgaggcacagagaggttaagtaacttgcccaaggtcacacagc

SEQ ID NO: 6 - DNA, 73 bp, Artificial Sequence
  MIRc reverse-complement
ttattatccccattttacagatgaggaaactgaggctcagagaggttaagtgacttgcccaaggtcacacagc

SEQ ID NO: 7 - DNA, 73 bp, Artificial Sequence; identical to SEQ ID NO: 6
  MIRb reverse complement

SEQ ID NO: 8 - DNA, 65 bp, Artificial Sequence
  reverse-complement of the Gilbert & Labuda 65-bp CORE-SINE sequence
  misc_feature (42)..(42): r=a or g
  misc_feature (44)..(44): r=a or g
cccattttacagatgaggaaactgaggctcagagaggttaartracttgcccaaggtcacacagc

SEQ ID NO: 9 - DNA, 65 bp, Artificial Sequence
  65-bp CORE-SINE sequence described by Gilbert & Labuda
  misc_feature (22)..(22): y=c or t
  misc_feature (24)..(24): y=c or t
gctgtgtgaccttgggcaagtyayttaacctctctgagcctcagtttcctcatctgtaaaatggg

SEQ ID NO: 10 - DNA, 73 bp, Artificial Sequence
  direct sequence for MIRc
gctgtgtgaccttgggcaagtcacttaacctctctgagcctcagtttcctcatctgtaaaatggggataataa

SEQ ID NO: 11 - RNA, 449 nt, Artificial Sequence
  t-NAT1
gccccagucugcggagagggagggcgaggggcggcggcgcaggggugcacagaggcggacggcgaggcagauuucggagccgcggcgcuuaccugauagucgacagaggcgaggacgggagaggacagcggaggaggagaagguggcugugguggcggcggcagaaggagucagaacaaggacgggccugugaugugucugucuaugacgaggaggacaauguccuaaggaauggagaggaagaaagugaagggaaccaggucccuggauuccauguggaacaguagcccaggaugugcacuauggacuguuacaagagagagacaugaucuuccuuaaggcauugauuugucaugagucucuuuguuauagccacuuaacguuaccuuaauacacccacuucauggauaaguaaaacugaggcuuggagaagcccacauugacacagc

SEQ ID NO: 12 - RNA, 544 nt, Artificial Sequence
  t-NAT2L
gcggccgccgaguccguccacaucgccaggccaggaccuuggacagggcccagcacaggggcccuggaauguggacugucucaguggauucuuguuuauaggaauuagaggaagguggaagaagcucauuccagagugcaaucaauaacaguggaaagaagucccuggagcccagugcaguggcuugaacugagaaugcacucacuggcuguugcagccaagacuccaguucucgccucucuuucccugacuccccugugcccaccuuuccccugcaggagucagaacaaggacgggccugugaugugucugucuaugacgaggaggacaauguccuaaggaauggagaggaagaaagugaagggaaccaggucccuggauuccauguggaacaguagcccaggaugugcacuauggacuguuacaagagagagacaugaucuuccuuaaggcauugauuugucaugagucucuuuguuauagccacuuaacguuaccuuaauacacccacuucauggauaaguaaaacugaggcuuggagaag

SEQ ID NO: 13 - RNA, 924 nt, Artificial Sequence
  t-Nat2s
agaaagaaauccgccccgagaugcaccugcagccccgcgcccauccgugcguggcugcggcugugcgugcccgcgaacggggaccagcggccgccgaguccguccacaucgccaggccaggagucagaacaaggacgggccugugaugugucugucuaugacgaggaggacaauguccuaaggaauggagaggaagaaagugaagggaaccaggucccuggauuccauguggaacaguagcccaggaugugcacuauggacuguuacaagagagagacaugaucuuccuuaaggcauugauuugucaugagucucuuuguuauagccacuuaacguuaccuuaauacacccacuucauggauaaguaaaacugaggcuuggagaagcccacauugacacagcaaagaaggcuggggcucuugugcugguuaaauggaccuugaugucuuccaaauguuuucaugcuggauucugagccacucuccuggcugcaacucugaggccgggcagacuuuccgcuggaaagagaacucaaauacuggugagagguggauuugaagccaggagccccagguuuuggcucugcagguuugcacccagcucugugguguaugcccucacaggucacccaggaccuccuguccaggcuucuucaggccacccaugauggaguagauuugaggaaugagguuuucaaagcaguuagugugcgggagaguaacccuggggccuccggaaccagaagggagggauuugggggcuggagcuuggcaguccagguuccauuguccucauguucuccccacuggccuggccugucacuccagucucugcauagguugucacauggcauucucccugugugucucugugucucuucucuucuuuauaaagacauaguuauaauggauuagggcccacccu

SEQ ID NO: 14 - DNA, 75 bp, Artificial Sequence
  t-NAT1 that overlaps with MAPT non-coding exon (-1)
ctgatagtcgacagaggcgaggacgggagaggacagcggaggaggagaaggtggctgtggtggcggcggcagaag

SEQ ID NO: 15 - DNA, 92 bp, Artificial Sequence
  t-NAT1 that overlaps with MAPT intron -1
gccccagtctgcggagagggagggcgaggggcggcggcgcaggggtgcacagaggcggacggcgaggcagatttcggagccgcggcgcttac

SEQ ID NO: 16 - DNA, 34 bp, Artificial Sequence
  t-NAT21 that overlaps with MAPT intron (-1)
gcggccgccgagtccgtccacatcgccaggccag

SEQ ID NO: 17 - DNA, 120 bp, Artificial Sequence
  t-NAT2s that overlaps with MAPT intron (-1)
agaaagaaatccgccccgagatgcacctgcagccccgcgcccatccgtgcgtggctgcggctgtgcgtgcccgcgaacggggaccagcggccgccgagtccgtccacatcgccaggccag

SEQ ID NO: 18 - RNA, 1869 nt, Artificial Sequence
  18S rRNA sequence
uaccugguugauccugccaguagcauaugcuugucucaaagauuaagccaugcaugucugaguacgcacggccgguacagugaaacugcgaauggcucauuaaaucaguuaugguuccuuuggucgcucgcuccucuccuacuuggauaacugugguaauucuagagcuaauacaugccgacgggcgcugacccccuucgcgggggggaugcgugcauuuaucagaucaaaaccaacccggucagccccucuccggccccggccggggggcgggcgccggcggcuuuggugacucuagauaaccucgggccgaucgcacgccccccguggcggcgacgacccauucgaacgucugcccuaucaacuuucgaugguagucgccgugccuaccauggugaccacgggugacggggaaucaggguucgauuccggagagggagccugagaaacggcuaccacauccaaggaaggcagcaggcgcgcaaauuacccacucccgacccggggagguagugacgaaaaauaacaauacaggacucuuucgaggcccuguaauuggaaugaguccacuuuaaauccuuuaacgaggauccauuggagggcaagucuggugccagcagccgcgguaauuccagcuccaauagcguauauuaaaguugcugcaguuaaaaagcucguaguuggaucuugggagcgggcgggcgguccgccgcgaggcgagccaccgcccguccccgccccuugccucucggcgcccccucgaugcucuuagcugagugucccgcggggcccgaagcguuuacuuugaaaaaauuagaguguucaaagcaggcccgagccgccuggauaccgcagcuaggaauaauggaauaggaccgcgguucuauuuuguugguuuucggaacugaggccaugauuaagagggacggccgggggcauucguauugcgccgcuagaggugaaauucuuggaccggcgcaagacggaccagagcgaaagcauuugccaagaauguuuucauuaaucaagaacgaaagucggagguucgaagacgaucagauaccgucguaguuccgaccauaaacgaugccgaccggcgaugcggcggcguuauucccaugacccgccgggcagcuuccgggaaaccaaagucuuuggguuccggggggaguaugguugcaaagcugaaacuuaaaggaauugacggaagggcaccaccaggaguggagccugcggcuuaauuugacucaacacgggaaaccucacccggcccggacacggacaggauugacagauugauagcucuuucucgauuccgugggugguggugcauggccguucuuaguugguggagcgauuugucugguuaauuccgauaacgaacgagacucuggcaugcuaacuaguuacgcgacccccgagcggucggcgucccccaacuucuuagagggacaaguggcguucagccacccgagauugagcaauaacaggucugugaugcccuuagauguccggggcugcacgcgcgcuacacugacuggcucagcgugugccuacccuacgccggcaggcgcggguaacccguugaaccccauucgugauggggaucggggauugcaauuauuccccaugaacgaggaauucccaguaagugcgggucauaagcuugcguugauuaagucccugcccuuuguacacaccgcccgucgcuacuaccgauuggaugguuuagugaggcccucggaucggccccgccggggucggcccacggcccuggcggagcgcugagaagacggucgaacuugacuaucuagaggaaguaaaagucguaacaagguuuccguaggugaaccugcggaaggaucauua

SEQ ID NO: 19 - RNA, 432 nt, Artificial Sequence
  18S rRNA sequence active region
aauuagaguguucaaagcaggcccgagccgccuggauaccgcagcuaggaauaauggaauaggaccgcgguucuauuuuguugguuuucggaacugaggccaugauuaagagggacggccgggggcauucguauugcgccgcuagaggugaaauucuuggaccggcgcaagacggaccagagcgaaagcauuugccaagaauguuuucauuaaucaagaacgaaagucggagguucgaagacgaucagauaccgucguaguuccgaccauaaacgaugccgaccggcgaugcggcggcguuauucccaugacccgccgggcagcuuccgggaaaccaaagucuuuggguuccggggggaguaugguugcaaagcugaaacuuaaaggaauugacggaagggcaccaccaggaguggagccuaaggaucauua

SEQ ID NO: 20 - RNA, 7 nt, Artificial Sequence
  RNA motif
cacccac

SEQ ID NO: 21 - RNA, 7 nt, Artificial Sequence
  RNA motif
cugaggc

SEQ ID NO: 22 - RNA, 9 nt, Artificial Sequence
  RNA motif
aacugaggc

SEQ ID NO: 23 - DNA, 7 bp, Artificial Sequence
  DNA motif
ctgaggc

SEQ ID NO: 24 - RNA, 8 nt, Artificial Sequence
  RNA motif
aacgaggc

SEQ ID NO: 25 - DNA, 8 bp, Artificial Sequence
  DNA motif
cctcccct

SEQ ID NO: 26 - DNA, 8 bp, Artificial Sequence
  DNA motif
aataaaat

SEQ ID NO: 27 - DNA, 28 bp, Artificial Sequence
  NT1-5′ F (BamHI)
ggcggatccgccccagtctgcggagagg

SEQ ID NO: 28 - DNA, 29 bp, Artificial Sequence
  NT1 3′ R (XhoI)
ggcctcgaggctgtgtcaatgtgggcttc

SEQ ID NO: 29 - DNA, 27 bp, Artificial Sequence
  TOPO2-F (EcoRV)
ggcgatatcccatgaagtgggtgtatt

SEQ ID NO: 30 - DNA, 29 bp, Artificial Sequence
  TOPO2-R (BamHI)
ggcggatcccgagtccgtccacatcgcca

SEQ ID NO: 31 - DNA, 22 bp, Artificial Sequence
  B1b-MluI-F
gtcacgcgtgagtgcgcgcgtg

SEQ ID NO: 32 - DNA, 28 bp, Artificial Sequence
  B2-F-NheI
agtgctagctgccgctgttcgccatcag

SEQ ID NO: 33 - DNA, 31 bp, Artificial Sequence
  B2-R-EcoRV
tagccgatatcaccctcagaataaaagccag

SEQ ID NO: 34 - DNA, 29 bp, Artificial Sequence
  B1F-NruI
ccttcgcgacaaatgctctgcgatgtgtt

SEQ ID NO: 35 - DNA, 29 bp, Artificial Sequence
  B1R-HindIII
cctaagcttggacagcggatttcagattc

SEQ ID NO: 36 - DNA, 27 bp, Artificial Sequence
  NT1 delta 5-BamHI
gaaggatccgagtcagaacaaggacgg

SEQ ID NO: 37 - DNA, 27 bp, Artificial Sequence
  NT1 delta 5-XhoI
gaactcgagttgctgtgtcaatgtggg

SEQ ID NO: 38 - DNA, 26 bp, Artificial Sequence
  NT1 delta 3-BamHI
gaaggatccagtctgcggagagggag

SEQ ID NO: 39 - DNA, 24 bp, Artificial Sequence
  NT1 delta 3-XhoI
gaactcgagcttctgccgccgcca

SEQ ID NO: 40 - DNA, 81 bp, Artificial Sequence
  NT1overS
gatcccttctgccgccgccaccacagccaccttctcctcctccgctgtcctctcccgtcctcgcctctgtcgactatcagc

SEQ ID NO: 41 - DNA, 81 bp, Artificial Sequence
  NT1overAS
ggaagacggcggcggtggtgtcggtggaagaggaggaggcgacaggagagggcaggagcggagacagctgatagtcgagct

SEQ ID NO: 42 - DNA, 81 bp, Artificial Sequence
  NT1overFlipS
gatccctgatagtcgacagaggcgaggacgggagaggacagcggaggaggagaaggtggctgtggtggcggcggcagaagc

SEQ ID NO: 43 - DNA, 81 bp, Artificial Sequence
  NT1overFlipAS
ggactatcagctgtctccgctcctgccctctcctgtcgcctcctcctcttccaccgacaccaccgccgccgtcttcgagct

SEQ ID NO: 44 - DNA, 93 bp, Artificial Sequence
  NT1nonoverS
gatccagtctgcggagagggagggcgaggggcggcggcgcaggggtgcacagaggcggacggcgaggcagatttcggagccgcggcgcttacc

SEQ ID NO: 45 - DNA, 93 bp, Artificial Sequence
  NT1nonoverAS
gtcagacgcctctccctcccgctccccgccgccgcgtccccacgtgtctccgcctgccgctccgtctaaagcctcggcgccgcgaatggagct

SEQ ID NO: 46 - DNA, 27 bp, Artificial Sequence
  NT2 delta 5F-BamHI
gaaggatccgagtcagaacaaggacgg

SEQ ID NO: 47 - DNA, 31 bp, Artificial Sequence
  NT2 delta 5R-XhoI
gaactcgagccatgaagtgggtgtattaagg

SEQ ID NO: 48 - DNA, 25 bp, Artificial Sequence
  NT2 delta 3F-BamHI
gaaggatccccgagtccgtccacat

SEQ ID NO: 49 - DNA, 25 bp, Artificial Sequence
  NT2 delta 3R-XhoI
gaactcgagctgcaggggaaaggtg

SEQ ID NO: 50 - DNA, 47 bp, Artificial Sequence
  NT1 delta mir1-XhoI
gaactcgagttgctgtgtcaatgtgggggtaacgttaagtggctata

SEQ ID NO: 51 - DNA, 29 bp, Artificial Sequence
  NT1 delta mir2-XhoI
gaactcgagggtaacgttaagtggctata

SEQ ID NO: 52 - DNA, 41 bp, Artificial Sequence
  NT2 delta mirR-XhoI
gaactcgagggtaacgttaagtggctataacaaagagactc

SEQ ID NO: 53 - DNA, 29 bp, Artificial Sequence
  B1LucF-BamHI
gaaggatcccttggcaatccggtactgtt

SEQ ID NO: 54 - DNA, 29 bp, Artificial Sequence
  B1LucR-HindIII
gaaaagcttccgtcttcgagtgggtagaa

SEQ ID NO: 55 - DNA, 29 bp, Artificial Sequence
  B1LucF-HindIII
gaaaagcttcttggcaatccggtactgtt

SEQ ID NO: 56 - DNA, 29 bp, Artificial Sequence
  B1LucR-BamHI
gaaggatccccgtcttcgagtgggtagaa

SEQ ID NO: 57 - DNA, 26 bp, Artificial Sequence
  CP-F
gagctccaaatgctctgcgatgtgtt

SEQ ID NO: 58 - DNA, 26 bp, Artificial Sequence
  CP-R
gctagcggacagcggatttcagattc

SEQ ID NO: 59 - DNA, 27 bp, Artificial Sequence
  NP-F
gagctagctgccgctgttcgccatcag

SEQ ID NO: 60 - DNA, 28 bp, Artificial Sequence
  NP-R
gtgctagcaccctcagaataaaagccag

SEQ ID NO: 61 - DNA, 23 bp, Artificial Sequence
  Fr1-F
gaggtccctggggcggtcaataa

SEQ ID NO: 62 - DNA, 25 bp, Artificial Sequence
  Fr1-R
aagcttaggcagtgattgggctctc

SEQ ID NO: 63 - DNA, 24 bp, Artificial Sequence
  FR2-F
gagctcgtagggggctgagttgag

SEQ ID NO: 64 - DNA, 26 bp, Artificial Sequence
  Fr2-R
aagcttaccagaagtggcagaattgg

SEQ ID NO: 65 - DNA, 26 bp, Artificial Sequence
  Fr3-F
gagctccagactgggttcctctccaa

SEQ ID NO: 66 - DNA, 24 bp, Artificial Sequence
  Fr3-R
aagcttgccagcatcacaaagaag

SEQ ID NO: 67 - DNA, 20 bp, Artificial Sequence
  RVp3-F
tagcaaaataggctgtcccc

SEQ ID NO: 68 - DNA, 19 bp, Artificial Sequence
  Luc-R
atgtgcgtcggtaaaggcg

SEQ ID NO: 69 - DNA, 27 bp, Artificial Sequence
  pRTF-EcoRI
ggtttgaattcggacggccgagcggca

SEQ ID NO: 70 - DNA, 47 bp, Artificial Sequence
  pRTF-NcoI
taaccatggcctggttcaaagttcacctgatagtcgacagaggcgag

SEQ ID NO: 71 - DNA, 28 bp, Artificial Sequence
  pRTFDover-EcoRI
ggtttgaattcccttctgccgccgccac

SEQ ID NO: 72 - DNA, 24 bp, Artificial Sequence
  pRTFDover-NcoI
taaccatggtgggcggtggcagcg

SEQ ID NO: 73 - DNA, 40 bp, Artificial Sequence
  pRTF-mTOP
ccgcgctgcccgcccaataaaatggggaggctcgcgttcc

SEQ ID NO: 74 - DNA, 28 bp, Artificial Sequence
  pRTFDover-PvuII
ggtttcagctgccttctgccgccgccac

SEQ ID NO: 75 - DNA, 23 bp, Artificial Sequence
  pRFseq-F
gaaggtgccaagaagtttcctaa

SEQ ID NO: 76 - DNA, 21 bp, Artificial Sequence
  siNT1nover S
cggcgaggcagauuucggatt

SEQ ID NO: 77 - DNA, 21 bp, Artificial Sequence
  siNT1nover AS
uccgaaaucugccucgccgtc

SEQ ID NO: 78 - DNA, 21 bp, Artificial Sequence
  siNT2nover S
gccgccgaguccguccacatt

SEQ ID NO: 79 - RNA, 21 nt, Artificial Sequence
  siNT2nover AS
uguggacggacucggcggccg

SEQ ID NO: 80 - DNA, 21 bp, Artificial Sequence
  siEx4-n268302 S
aggacaauguccuaaggaatt

SEQ ID NO: 81 - RNA, 21 nt, Artificial Sequence
  siEx4-n268302 AS
uuccuuaggacauuguccucc

SEQ ID NO: 82 - DNA, 21 bp, Artificial Sequence
  siEx4-n268298 S
gauuugucaugagucucuutt

SEQ ID NO: 83 - RNA, 21 nt, Artificial Sequence
  siEx4-n268298 AS
aagagacucaugacaaaucaa

SEQ ID NO: 84 - DNA, 268 bp, Artificial Sequence
  MIRc
aataataataaccccttacatttgtatagcactttacagtttacaaagcgctttcacatacattatctcatttgatcctcacaacaaccctgtgaggtaggcagggcaggtattattatccccattttacagatgaggaaactgaggctcagagaggttaagtgacttgcccaaggtcacacagctagtaagtggcagagccaggactcgaacccaggtcttcctgactccaagtccagtgctctttccactgcaccacactgcctcg

SEQ ID NO: 85 - DNA, 268 bp, Artificial Sequence
  MIRb
aataatagagctaccatttattgagcgcttactgtgtgccaggcactgtgctaagcgctttacatgcattatctcatttaatcctcacaacaaccctgcgaggtaggtattattatccccattttacagatgaggaaactgaggctcagagaggttaagtgacttgcccaaggtcacacagctagtaagtggcagagccaggattcgaacccaggtctgcctgactccaaagcccgtgctctttccactgcaccacgctgcccctctg

SEQ ID NO: 86 - DNA, 260 bp, Artificial Sequence
  MIR
taataaccaacatttattgagcgcttactatgtgccaggcactgttctaagcgctttacatgtattaactcatttaatcctcacaacaaccctatgaggtaggtactattattatccccattttacagatgaggaaactgaggcacagagaggttaagtaacttgcccaaggtcacacagctagtaagtggcagagccgggattcgaacccaggcagtctggctccagagtccgtgctcttaaccactatgctatactgt

SEQ ID NO: 87 - DNA, 224 bp, Artificial Sequence
  MIR3
  misc_feature (71)..(71): n=a,c,g or t
tttttttagaatcatagaatcatagaatgttagagctggaagggaccttagagatcatctagtccaacccnctcattttacagatgaggaaaactgaggcccagagaggtgaagtgacttgcccaaggtcacacagctagttagtggcagagctaggactagaacccaggtctcctgactcctagtccagttgttctttccactataccatactgcttccagaa

(19) United States
(12) Patent Application Publication
(10) Pub. No.: US 2019/0314398 A1
(43) Pub. Date: Oct. 17, 2019

(54) MEANS FOR MODULATING GENE EXPRESSION

(71) Applicant: UCL BUSINESS PLC, London Greater London (GB)

(72) Inventors: Roberto SIMONE, London Greater London (GB); Rohan DE SILVA, London Greater London (GB)

(21) Appl. No.: 16/303,094

(22) PCT Filed: May 19, 2017
(86) PCT No.: PCT/GB2017/051397

§ 371 Date: Nov. 19, 2018

(30) Foreign Application Priority Data
May 20, 2016 (GB) 1608907.0

(51) Int. Cl. A61K 31/7105; A61P 25/28; C12N 15/113

(52) U.S. Cl. CPC A61K 31/7105; C12N 15/113; A61P 25/28

(57) ABSTRACT

The present invention provides vectors for delivering to a cell, or expressing in a cell, a therapeutic RNA that is capable of reducing expression of a target gene. Compositions comprising the vectors and comprising the therapeutic RNAs are also provided, as are methods for their use.

### FIELD OF THE INVENTION

The present invention relates to means for modulating gene expression and, in particular, means for suppressing gene expression. In particular, the invention relates to the suppression of genes associated with neurological disorders, e.g. the MAPT gene that expresses tau protein.

### BACKGROUND OF THE INVENTION

Long non-coding RNA Mammalian genomes are pervasively transcribed, producing a vast array of transcripts with a wide range of size and coding potential^{1,2,3,4}. This includes many thousands of long non-coding RNAs (lncRNA), some of which are antisense relative to protein-coding genes (AS-lncRNAs). It has been shown that AS-lncRNA can regulate the chromatin state, transcription, RNA stability, and translation of the gene^{5,6}.

### Tau Protein

The MAPT gene expresses the microtubule-associated tau protein, which is associated with a large class of neurodegenerative diseases, collectively known as tauopathies. Tau is primarily expressed in the nervous systems, where it is involved in the dynamic stabilization of the axonal microtubule network. Expression and splicing of MAPT gene is developmentally regulated, with six isoforms expressed in the adult CNS. These consist of equal ratios of isoforms with three-(3R-tau) and four-(4R-tau) MT-binding repeat domains.

Fibrillar aggregates of abnormally hyperphosphoylated tau form the pathological hallmarks of a diverse class of neurodegenerative disorders called tauopathies, including Alzheimer's disease (AD), frontotemporal dementia (FTLD-tau), progressive supranuclear palsy (PSP) and corticobasal degeneration (CBD). Mutations in MAPT cause familial FTLD-tau⁷, and common variation in the form of the MAPT H1 haplotype are significant risk factors in PSP⁸, CBD⁹ and Parkinson's disease (PD)¹⁰. These genetic factors contribute to disruptions in tau isoform homeostasis or impaired MT-binding, resulting in increased levels of aggregation-prone unbound cytoplasmic tau.

Recent studies in different tauopathy mouse models have shown that conditional knockout or reduction of tau levels halt progression of tau pathology with behavioral improvements^{11,12,13}. Studies of the role of the MAPT H1 haplotype suggest increased expression and altered splicing, although little is yet known of the molecular mechanisms governing the physiological regulation of MAPT expression.

### SUMMARY OF THE INVENTION

The present inventors have characterised MAPT-AS1, a lncRNA gene that is antisense to the human MAPT gene and they have identified, for the first time, RNA transcripts of MAPT-AS1, which were found to inhibit MAPT expression. Furthermore, the present inventors have found that this inhibition of MAPT expression occurs at the stage of tau translation, not transcription. The inventors have also identified regions of the MAPT-AS1 lncRNA transcripts that mediate translational repression: They find that the presence of a MIR element in a reverse orientation is required for repressive activity, and that the deletion or inversion of the MIR element can lead instead to enhancement of MAPT expression, rather than repression.

The present inventors have also extended their findings from MAPT to other genes that have associated AS-lncRNAs. The inventors have shown that RNA molecules, which have sequences that correspond with an AS-lncRNA, can modulate the expression of a target gene that is associated with the AS-lncRNA. The inventors have experimentally validated their findings, demonstrating the modulation of expression of proteins such as tau protein.

Accordingly, at its broadest, the invention relates to a therapeutic RNA molecule that comprises one or more nucleotide sequences that correspond with an antisense long non-coding RNA, which therapeutic RNA molecule can modulate expression of a target gene.

In a first aspect, the invention provides a therapeutic RNA that is capable of reducing expression of a target gene,
wherein the therapeutic RNA comprises one or more nucleotide sequences that correspond with an antisense long non-coding RNA (AS-lncRNA), wherein the AS-lncRNA comprises a MIR domain in inverse orientation and wherein the AS-lncRNA is encoded by a genomic DNA sequence that is antisense to the target gene, and
wherein the therapeutic RNA comprises a sequence that corresponds with the MIR domain. The target gene may express tau protein.

In a second aspect, the invention provides a therapeutic RNA that is capable of enhancing expression of a target gene,
wherein the therapeutic RNA comprises one or more nucleotide sequences that correspond with an antisense long non-coding RNA (AS-lncRNA), wherein the AS-lncRNA comprises a MIR domain in direct orientation and wherein the AS-lncRNA is encoded by a genomic DNA sequence that is antisense to the target gene, and
wherein the therapeutic RNA comprises a sequence that corresponds with the MIR domain.

In a third aspect, the invention provides a vector for delivering to a cell, or expressing in a cell, a therapeutic RNA,
wherein the therapeutic RNA is capable of reducing expression of a target gene,
wherein the therapeutic RNA comprises one or more nucleotide sequences that correspond with an antisense long non-coding RNA (AS-lncRNA), wherein the AS-lncRNA comprises a MIR domain in inverse orientation and wherein the AS-lncRNA is encoded by a genomic DNA sequence that is antisense to the target gene, and
wherein the therapeutic RNA comprises a sequence that corresponds with the MIR domain. The target gene may express tau protein.

In a fourth aspect, the invention provides a vector for delivering to a cell, or expressing in a cell, a therapeutic RNA,
wherein the therapeutic RNA is capable of enhancing expression of a target gene,
wherein the therapeutic RNA comprises one or more nucleotide sequences that correspond with an antisense long non-coding RNA (AS-lncRNA), wherein the AS-lncRNA comprises a MIR domain in direct orientation and wherein the AS-lncRNA is encoded by a genomic DNA sequence that is antisense to the target gene, and
wherein the therapeutic RNA comprises a sequence that corresponds with the MIR domain.

In some embodiments of the invention (e.g., embodiments of the first, second, third and/or fourth aspect of the invention), the genomic sequence encoding the AS-lncRNA overlaps with the genomic sequence of the target gene. In some embodiments, the genomic sequence encoding the AS-lncRNA overlaps with the genomic sequence of an intron of the target gene. The part of the AS-lncRNA genomic sequence that overlaps with the genomic sequence encoding the target gene may be an exon at the 5′ end of the AS-lncRNA.

In some embodiments, the AS-lncRNA comprises an exon at the 5′ end of the AS-lncRNA that overlaps with the target gene and wherein the therapeutic RNA comprises a nucleotide sequence that corresponds with the exon at the 5′ end of the AS-lncRNA. In some embodiments, the exon at the 5′ end of the AS-lncRNA overlaps at least partially with the 5′ UTR of the target gene. The exon at the 5′ end of the AS-lncRNA may overlap at least partially with an intron of the target gene, including in those embodiments in which the exon at the 5′ end of the AS-lncRNA partially overlaps with the 5′ UTR of the target gene. In some embodiments, the exon at the 5′ end of the AS-lncRNA overlaps at least partially with an exon encoding the target gene. The target gene may be selected from the group consisting of the target genes listed in Table 1. In some embodiments, the target gene is selected from the group consisting of MAPT, SNCA, APP, MBNL1, SLC1A2, TPP1, DHCR24, ECE1, IMMT, FADD, MATR3, CDKN2A, DDX20, UCHL1, PRPH, GARS, DCTN1, ZNF224, KLK6, BDNF, PPP3CB, CELF1 and DERL1.

In some embodiments, the sequence of the therapeutic RNA that corresponds with the MIR domain comprises a nucleotide sequence having at least 70% identity to a portion of the MIR domain of any one of SEQ ID NOs: 1-10 that is able to drive modulation of expression of the target gene, wherein sequence identity is determined across the full length of the portion. For example, a therapeutic RNA that suppresses target gene expression may have a sequence that has at least 70% identity to a portion of the MIR domain of any one of SEQ ID NOs: 1-8, whereas a therapeutic RNA that enhances target gene expression may have a sequence that has at least 70% identity to a portion of the MIR domain of any one of SEQ ID NOs: 9 or 10.

In some embodiments, the sequence of the therapeutic RNA that corresponds with the MIR domain comprises a ‘CACCCAC’ and/or a ‘CUGAGGC’ motif.

In some embodiments of the invention (e.g. embodiments of the third or fourth aspects), the vector comprises a cDNA which encodes the therapeutic RNA. The vector may be a plasmid vector or the vector may be a viral vector comprising the cDNA, such as an AAV vector. Where the vector is a plasmid vector, it may be associated with a nanoparticle, a dendrimer, a polyplex, a liposome, a micelle or a lipoplex. In other embodiments of the invention (e.g. other embodiments of the third or fourth aspects), the vector comprises the therapeutic RNA itself. The vector may be a nanoparticle, a dendrimer, a polyplex, a liposome, a micelle or a lipoplex.

In a fifth aspect, the invention provides the therapeutic RNA of the first or second aspect, or the vector of the third or fourth aspect for use in methods of treating the human or animal body by therapy. Said methods of treating the human or animal body are hereby disclosed.

In a sixth aspect, the invention provides the therapeutic RNA of the first or second aspect, or the vector of the third or fourth aspect for use in methods of treating a neurodegenerative condition in a subject, wherein the methods comprise administering the therapeutic RNA or the vector to the subject. Said methods of treating neurodegenerative conditions are hereby disclosed.

In some embodiments, the neurodegenerative condition is a tauopathy, such as Alzheimer's disease. In some embodiments, the neurodegenerative condition is Parkinson's disease.

In a seventh aspect, the invention provides a method of producing a genetically engineered organism, the method comprising introducing the MAPT-AS1 gene into one or more cells of an organism to produce the genetically engineered organism.

In an eighth aspect, the invention provides genetically engineered organisms that have one or more additional copies of the MAPT-AS1 gene. The genetically engineered organisms have one or more additional copies of the MAPT-AS1 gene compared with an equivalent organism that is not engineered to have one or more additional copies of the MAPT-AS1 gene. In some embodiments of the eighth aspect, the equivalent organism does not have an endogenous copy of the MAPT-AS1 gene.

In a ninth aspect, the invention provides a method of producing a lncRNA that is capable of modulating the expression of a protein-coding gene, the method comprising;
(a) identifying a population of genes that encode a lncRNA, wherein each member of the population comprises a sequence that overlaps a 5′ untranslated region (UTR), an intron, a coding sequence (CDS), and/or a 3′ UTR of a protein-coding gene, and wherein each member of the population is in antisense orientation with respect to the respective protein-coding gene,
(b) identifying members of the population of genes that encode a lncRNA identified in step (a) that comprise a MIR domain,
(c) selecting a gene from the population identified in (b), and
(d) causing or allowing a transcript of the selected gene to be expressed, which transcript is the produced lncRNA. In some embodiments, steps (a) and/or (b) and/or (c) may be performed in silico, e.g. by using a computer-implemented program which is run on a computer.

The modulation may be suppression of expression of the respective protein-coding gene that overlaps the gene that encodes the lncRNA if the MIR domain of the lncRNA is in inverse orientation, or the modulation may be enhancement of expression of the respective protein-coding gene that overlaps the gene that encodes the lncRNA if the MIR domain of the lncRNA is in direct orientation.

In some embodiments, a further step of isolating the produced lncRNA may be performed. In some embodiments of the ninth aspect, further steps of determining the minimum portions of the lncRNA that are required to modulate expression of the protein-coding (target) gene may be performed and yet further steps including the production of a cDNA encoding only the minimum portions may also be performed.

In a tenth aspect, the invention provides a method of selecting a target gene by
(a) identifying a population of genes that encode a lncRNA, wherein each member of the population comprises a sequence that overlaps a 5′ untranslated region (UTR), an intron, a coding sequence (CDS), and/or a 3′ UTR of a protein-coding gene, and wherein each member of the population is in antisense orientation with respect to the respective protein-coding gene,
(b) identifying members of the population of genes that encode a lncRNA identified in step (a) that comprise a MIR domain,
(c) selecting the target gene from a population of protein coding genes that comprise a 5′ untranslated region (UTR), an intron, a coding sequence (CDS), and/or a 3′ UTR that overlap with a member of the population of genes that encode a lncRNA and comprise a MIR domain, identified in step (b) of claim 42. In some embodiments, steps (a) and/or (b) and/or (c) may be performed in silico, e.g. by using a computer-implemented program.

In some embodiments, expression of the target gene is identified as being susceptible to being suppressed by a therapeutic RNA if the MIR domain of the overlapping lncRNA gene is in inverse orientation, or wherein the expression of the target gene is identified as being susceptible to being enhanced by a therapeutic RNA if the MIR domain of the overlapping lncRNA gene is in direct orientation. The target gene may be associated with a neurodegenerative disease.

In some embodiments, the methods further comprise a step of providing a therapeutic RNA molecule comprising one or more sequences that correspond with one or more sequences of the overlapping lncRNA and may also comprise a step of modulating the expression of the target gene by contacting a cell comprising the target gene with a therapeutic RNA.

Kits for performing the methods of the invention are also disclosed.

### Therapeutic RNA of the Invention

As described herein, the therapeutic RNA of the invention is capable of modulating expression of a target gene, and the therapeutic RNA comprises one or more nucleotide sequences that correspond with sequences of an antisense long non-coding RNA (AS-lncRNA). In some embodiments, the invention provides therapeutic RNA molecules that comprise only key functional domains of the AS-lncRNA (which may be termed ‘MININATs’). In other embodiments, the invention provides therapeutic RNA molecules that correspond with the entire length of an AS-lncRNA transcript. Intermediate configurations in which the therapeutic RNA corresponds with part- or most-of an AS-lncRNA transcript are also encompassed by the invention.

The therapeutic RNA of the invention modulates translation. The data disclosed herein suggests that the modulatory action is exerted at the ribosome and not in the nucleus. The therapeutic RNA of the invention may have advantages over conventional RNAi technologies such as siRNA, which are essentially restricted to act via the RISC complex located at P-bodies. The therapeutic RNA of the invention may e.g. exhibit higher potency than RNAi.

The therapeutic RNA of the invention finds uses in both in vivo and in vitro applications. In some embodiments of the invention, the therapeutic RNA is used in vivo. In some embodiments of the invention, the therapeutic RNA is used in vitro.

In some aspects, the therapeutic RNA of the invention comprises one or more nucleotide sequences that correspond with sequences of t-NAT1 (also denoted as tau-NAT1), which is a transcript of MAPT-AS1. In this aspect, the therapeutic RNA of the invention comprises a nucleotide sequence that corresponds with the MIR repeat domain in distal 3′-exon of MAPT-AS1. Preferably, in this aspect, the therapeutic RNA of the invention also comprises a nucleotide sequence that corresponds with the 5′ region of t-NAT1 that overlaps the 5′-untranslated region (5′-UTR; exon (−1)) of MAPT. In some embodiments of this aspect, the therapeutic RNA of the invention corresponds with the full-length t-NAT1 transcript. In other embodiments of this aspect, the therapeutic RNA of the invention corresponds with a functionally active truncated derivative of tau-NAT1 (denoted t-NAT1 MININAT).

In some aspects, the therapeutic RNA of the invention comprises one or more nucleotide sequences that correspond with sequences of t-NAT2L (also denoted tau-NAT2L), which is a transcript of MAPT-AS1. In this aspect, the therapeutic RNA of the invention comprises a nucleotide sequence that corresponds with the MIR repeat domain in distal 3′-exon of MAPT-AS1. Preferably, in this aspect, the therapeutic RNA of the invention also comprises a nucleotide sequence that corresponds with the 5′ exon of t-NAT2L, which overlaps with the first intron of MAPT. In some embodiments of this aspect, the therapeutic RNA of the invention corresponds with the full-length t-NAT2L transcript. In other embodiments of this aspect, the therapeutic RNA of the invention corresponds with a functionally active truncated derivative of tau-NAT2L (denoted t-NAT2L MININAT).

In some aspects, the therapeutic RNA of the invention comprises one or more nucleotide sequences that correspond with sequences of the transcript of a non-protein-coding gene that has a 5′-head-to-head sense-antisense overlapping sequence with a protein-coding gene, which non-protein-coding gene also has a distal MIR-repeat domain. In this aspect, the therapeutic RNA of the invention comprises a nucleotide sequence that corresponds with the MIR repeat domain. Preferably, in this aspect, the therapeutic RNA of the invention also comprises a nucleotide sequence that corresponds with a 5′ exon (or part of the 5′ exon) of the non-protein-coding gene that overlaps with the 5′ UTR of the protein-coding gene. In other embodiments of this aspect, the therapeutic RNA of the invention comprises a nucleotide sequence that corresponds with a 5′ exon (or the part of the 5′ exon) of the non-protein-coding gene that overlaps with an intron of the protein-coding gene. In some embodiments of this aspect, the therapeutic RNA of the invention corresponds with the full-length non-protein-coding transcript. In other embodiments of this aspect, the therapeutic RNA of the invention corresponds with a functionally active truncated derivative of the non-protein-coding.

### Degree of Correspondence

The following paragraphs describe the different degrees to which the therapeutic RNA of the invention may correspond with a lncRNA, for instance MIR-AS-lncRNAs such as t-NAT1 and t-NAT21.

In the context of this disclosure, two sequences are said to correspond with each other when they share a degree of sequence identity. The degree of sequence identity may be exactly 100% or less than 100%, e.g. at least 50%, 60%, 70%, 75%, 80%, 85%, 90%, 91%, 92%, 93%, 94%, 95%, 96%, 97%, 98%, 99% (wherein sequence identity is determined across the full length of either sequence). In the context of this disclosure, “at least 50%, 60%, 70%, 75%, etc . . . ” means “at least 50%, at least 60%, at least 70%, at least 75%, etc . . . ”. This definition of correspondence applies, for example to the correspondence between regions of the therapeutic RNA of the invention with the MIR domain of an lncRNA and to the correspondence between regions of the therapeutic RNA of the invention with the part of an lncRNA that overlaps with a target gene.

As well as sharing a degree of sequence identity, in the context of this disclosure, two sequences are said to correspond with each other when they are in the same orientation, irrespective of whether the sequence identity of the two sequences is 100% or less than 100%. Hence, in the context of this disclosure, two sequences correspond with each other when they are in the same orientation and they share a degree of sequence identity.

In some embodiments, the therapeutic RNA of the invention comprises (or consists of, or consists essentially of) a nucleotide sequence having having at least 50%, 60%, 70%, 75%, 80%, 85%, 90%, 91%, 92%, 93%, 94%, 95%, 96%, 97%, 98%, 99% or exactly 100% sequence identity to the MIR domain of a MIR-lncRNA (e.g. any one of SEQ ID NOs: 1-3), wherein sequence identity is determined across the full length of the MIR domain. In some embodiments, the MIR domain is that of a MAPT-AS1 transcript.

In some embodiments, the therapeutic RNA of the invention comprises (or consists of, or consists essentially of) a nucleotide sequence having having at least 50%, 60%, 70%, 75%, 80%, 85%, 90%, 91%, 92%, 93%, 94%, 95%, 96%, 97%, 98%, 99% or exactly 100% sequence identity to the CORE-SINE domain of one or more of the MIR sequences of classes MIR, MIR3, MIRb and MIRc (e.g. any one of SEQ ID NOs: 4-7 or 10), or of the CORE-SINE domain disclosed by Gilbert and Labuda (SEQ ID NOs: 8 or 9) wherein sequence identity is determined across the full length of the MIR domain. As disclosed herein, the orientation of the MIR domain determines whether the therapeutic RNA suppresses target gene expression (inverse orientation) or enhances target gene expression (direct orientation). In some embodiments, the MIR domain is that of a MAPT-AS1 transcript.

In some embodiments, the therapeutic RNA of the invention comprises (or consists of) a nucleotide sequence having at least 50%, 60%, 70%, 75%, 80%, 85%, 90%, 91%, 92%, 93%, 94%, 95%, 96%, 97%, 98%, 99% or exactly 100% sequence identity to a portion of the MIR domain of a class MIR, MIR3, MIRb or MIRc, or of a MIR-lncRNA, or of the CORE-SINE domain disclosed by Gilbert and Labuda, (e.g. any one of SEQ ID NOs: 1-10), which is able to drive repression or enhancement of expression of the target gene (e.g. a “minimum portion”), wherein sequence identity is determined across the full length of this portion.

The portion of the MIR domain which is able to drive repression or enhancement of expression of the target gene may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 nucleotides in length, or the portion may have a length between any two of these values. Alternatively, the portion may be at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 nucleotides in length. Using the disclosure of the present application, the skilled person is able to readily determine what portion of a given MIR domain is able to repress, or enhance, target gene expression. In some embodiments, the portion of the MIR domain is a portion of the MIR domain of a MAPT-AS1 transcript. The portion of a MIR domain which is able to drive repression or enhancement may comprise a “kmer” motif (for example a 7-mer as shown in Table 1) that corresponds with a kmer motif in the 5′-UTR of the target gene that is complementary to a motif in the 18S rRNA “active region” as defined by Weingarten-Gabbay et al²⁵ and by Petrov et al³⁵ (SEQ ID NO: 19). The portion of the MIR domain may comprise a 7-mer motif that is complementary or identical to a 7-mer motif in the active region of the human 18S rRNA sequence (SEQ ID NO: 19), which MIR domain motif may also find its complementary sequence an IRES of the target gene. The 7-mer motif in the IRES of the target gene may be complementary with a 7-mer motif of the 18S rRNA “active region”, e.g. at a position shown in FIG. 17. Hence, the therapeutic RNA may comprise a 7-mer motif that is itself identical to a motif in the “active region” of the human 18S rRNA. Additionally or alternatively, the therapeutic RNA may comprise a 7-mer motif that is complementary to a motif in the “active region” of the human 18S rRNA. In some embodiments, the portion may comprise ‘CUGAGGC’ or ‘AACUGAGGC’ and/or ‘CACCCAC’ motifs.

In some embodiments, the therapeutic RNA of the invention comprises a nucleotide sequence having having at least 50%, 60%, 70%, 75%, 80%, 85%, 90%, 91%, 92%, 93%, 94%, 95%, 96%, 97%, 98%, 99% or exactly 100% sequence identity to the 5′ region of t-NAT1 that (partially) overlaps the 5′-untranslated region (5′-UTR; also referred to as exon (−1)) of MAPT (i.e. SEQ ID NO: 14), wherein sequence identity is determined across the full length of SEQ ID NO: 14.

In some embodiments, the therapeutic RNA of the invention comprises a nucleotide sequence having having at least 50%, 60%, 70%, 75%, 80%, 85%, 90%, 91%, 92%, 93%, 94%, 95%, 96%, 97%, 98%, 99% or exactly 100% sequence identity to the 5′ region of t-NAT1 that (partially) overlaps the first intron of MAPT (also referred to as intron (−1)) (i.e. SEQ ID NO: 13), wherein sequence identity is determined across the full length of SEQ ID NO: 15.

In some embodiments, the therapeutic RNA of the invention comprises a nucleotide sequence having having at least 50%, 60%, 70%, 75%, 80%, 85%, 90%, 91%, 92%, 93%, 94%, 95%, 96%, 97%, 98%, 99% or exactly 100% sequence identity to either the 5′ exon of t-NAT21, which overlaps with the first intron of MAPT (i.e. SEQ ID NO: 16), or the 5′ exon of t-NAT2s, which overlaps with the first intron of MAPT (i.e. SEQ ID NO: 17), wherein sequence identity is determined across the full length SEQ ID NO: 16 or SEQ ID NO: 17.

In some embodiments, the therapeutic RNA of the invention comprises a nucleotide sequence having having at least 20%, 30%, 40%, 50%, 60%, 70%, 75%, 80%, 85%, 90%, 91%, 92%, 93%, 94%, 95%, 96%, 97%, 98%, 99% or exactly 100% sequence identity to an exon at the 5′ end of a MIR-AS-lncRNA that overlaps with an untranslated region or an intron or a coding sequence of a sense protein-coding gene, wherein sequence identity is determined across the full length of the exon at the 5′ end of the MIR-AS-lncRNA.

In some embodiments, the therapeutic RNA of the invention comprises a nucleotide sequence having having at least 50%, 60%, 70%, 75%, 80%, 85%, 90%, 91%, 92%, 93%, 94%, 95%, 96%, 97%, 98%, 99% or exactly 100% sequence identity to a portion of the exon at the 5′ end of the MIR-AS-lncRNA which is able to drive repression or enhancement of expression of the target gene, wherein sequence identity is determined across the full length of the portion of the exon at the 5′ end of the MIR-AS-lncRNA which is able to drive repression or enhancement of expression of the target gene.

The portion of the exon at the 5′ end of the MIR-AS-lncRNA which is able to drive repression or enhancement of expression of the target gene may overlap with the 5′ UTR of the target gene, and/or it may overlap with an intron of the target gene and/or it may overlap with a coding exon of the target gene. The nucleotide sequence corresponding with the portion of the exon at the 5′ end of the MIR-AS-lncRNA which is able to drive repression or enhancement of expression of the target gene may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 nucleotides in length, or the portion may have a length between any two of these values. Alternatively, the portion may be at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 nucleotides in length. In some embodiments, said portion may be a portion of SEQ ID NO: 13, SEQ ID NO: 14, SEQ ID NO: 15, or SEQ ID NO: 16. Said portion may comprise a 7-mer motif that is the reverse-complement of the 7-mer motif in the 5′-UTR of the target gene that itself is complementary with a motif of the 18S rRNA “active region” as defined by Weingarten-Gabbay et al²⁵ and by Petrov et al³⁵ (e.g. as shown in Table 1).

The complex folding of the 5′-UTR of the MAPT transcript leads to two main domains that together function as an internal ribosome entry site (IRES)²², providing the cis-acting signals for an alternative mode of translational regulation. In some embodiments, the therapeutic RNA of the invention comprises one or more sequences that correspond with sequences of an AS-lncRNA that interacts with one or more of the domains that function as an internal ribosome entry site (IRES). The therapeutic RNA of the invention may comprise one or more sequences that correspond with sequences of a MAPT-AS1 transcript that compete with or interact with the IRES in the 5′-UTR of the MAPT transcript.

In some embodiments, the therapeutic RNA of the invention comprises a nucleotide sequence having having at least 50%, 60%, 70%, 75%, 80%, 85%, 90%, 91%, 92%, 93%, 94%, 95%, 96%, 97%, 98%, 99% or exactly 100% sequence identity to a MIR-AS-lncRNA, wherein sequence identity is determined across the full length of the MIR-AS-lncRNA. The MIR-AS-lncRNA may be a transcript of MAPT-AS1, e.g. t-NAT1 (SEQ ID NO: 11) or t-NAT21 (SEQ ID NO: 12).

In some embodiments, the therapeutic RNA of the invention consists of, or consists essentially of, a nucleotide sequence having having at least 50%, 60%, 70%, 75%, 80%, 85%, 90%, 91%, 92%, 93%, 94%, 95%, 96%, 97%, 98%, 99% or exactly 100% sequence identity to a MIR-AS-lncRNA, wherein sequence identity is determined across the full length of the MIR-AS-lncRNA. The MIR-AS-lncRNA may be a transcript of MAPT-AS1, e.g. t-NAT1 (SEQ ID NO: 11) or t-NAT21 (SEQ ID NO: 12).

In some embodiments, the therapeutic RNA of the invention comprises a sequence that corresponds with the region of a transcript of MAPT-AS1 that overlaps with the MAPT core propmoter.

### MIR Domains

All three transcripts of MAPT-AS1 have a mammalian-wide interspersed repeat domain (MIR domain) at the 3′-end that corresponds with the reverse-complement of the CORE-SINE sequence described by Gilbert & Labuda (1999)¹⁹ (which reverse-complement is denoted SEQ ID NO: 8 herein). As noted herein, this MIR domain is thus described as being in the ‘inverted’ or ‘inverse’ orientation. The orientation of a MIR domain is therefore taken as being relative to the ‘direct’ CORE-SINE sequence described by Gilbert & Labuda (1999)¹⁹, which is denoted SEQ ID NO: 9 herein.

There is a high degree of conservation of the CORE-SINE in all MIR subclasses¹⁹ and the skilled person can readily identify the orientation of MIR domains (i.e. ‘inverted’ or ‘direct’) by sequence comparison. Online tools such as Repeatmasker can be utilized in this regard.

The MIR domain of MAPT-AS1 is highly conserved in all primates and it shows homology to the CORE-SINE sequence, common to all MIR repeats¹⁹ (FIG. 6d, 7d).

As noted herein, the MAPT-AS1 gene comprises a MIR domain. This is expressed in t-NAT21 as the following sequence:

[SEQ ID NO: 1]

UUAAUACACC CACUUCAUGG AUAAGUAAAA CUGAGGCUUG
GAGAAG

In t-NAT1 and t-NAT2s, the MIR domain is expressed as:

[SEQ ID NO: 2]

UUAAUACACC CACUUCAUGG AUAAGUAAAA CUGAGGCUUG
GAGAAGCCCA CAUUGACACA GC

The primate consensus sequence of the MAPT-AS1 MIR domain is:

[SEQ ID NO: 3]

UUAAUACACC CACUUCAUGG AUAAGUAAAA CUGAGGCUUG
GAGAAGCCCA CAUUGACACA GCA

In embodiments of the invention that use a therapeutic RNA to suppress target gene expression, the therapeutic RNA will include a MIR sequence in the inverse orientation, e.g. as shown above in SEQ ID NOs: 1-3. In embodiments that use a therapeutic RNA to enhance target gene expression, the therapeutic RNA will include a MIR sequence in the direct orientation, which will correspond with the reverse-complement of SEQ ID NOs: 1-3.

These MIR domains of the MAPT-AS1 transcripts have a high degree of homology the CORE-SINE elements of all MIR repeats (i.e., MIR3, MIR, MIRc and MIRb)¹⁹. In some embodiments of the invention, the therapeutic RNA comprises a MIR domain of subclass MIRc, or the therapeutic RNA comprises a MIR domain that corresponds with the sequence of MIRc.

The sequences of the CORE-SINE domain of MIR classes MIR, MIR3, MIRb, MIRc are shown here, as reverse-complement sequences (i.e. in the inverse orientation):

MIR3 reverse-complement:

[SEQ ID NO: 4]

CCAACCCNCTCATTTTACAGATGAGGAAAACTGAGGCCCAGAGAGGTGAA
GTGACTTGCCCAAGGTCACACAGC
MIR reverse-complement:

[SEQ ID NO: 5]

TTATTATCCCCATTTTACAGATGAGGAAACTGAGGCACAGAGAGGTTAAG
TAACTTGCCCAAGGTCACACAGC
MIRc reverse-complement:

[SEQ ID NO: 6]

TTATTATCCCCATTTTACAGATGAGGAAACTGAGGCTCAGAGAGGTTAAG
TGACTTGCCCAAGGTCACACAGC
MIRb reverse-complement:

[SEQ ID NO: 7]

TTATTATCCCCATTTTACAGATGAGGAAACTGAGGCTCAGAGAGGTTAAG
TGACTTGCCCAAGGTCACACAGC

The alignment between the above inverted MIR, MIR3, MIRb and MIRc sequences with the MIR domains of the DNA encoding t-NAT1 and t-NAT2s is shown in FIG. 6 and the alignment between the inverted MIR, MIR3, MIRb and MIRc sequences with the MIR domains of the DNA encoding and t-NAT21 is shown in FIG. 7.

The skilled person will appreciate that the ‘direct’ MIR sequences are unambiguously derivable from the above inverted sequences. For example, the direct sequence for MIRc is:

[SEQ ID NO: 10]

GCTGTGTGACCTTGGGCAAGTCACTTAACCTCTCTGAGCCTCAGTTTCCT
CATCTGTAAAATGGGGATAATAA

The 65-bp CORE-SINE sequence described by Gilbert & Labuda (1999)¹⁹ has the following (direct) sequence:

[SEQ ID NO: 9]

gctgtgtgaccttgggcaagtyayttaacctctctgagcctcagtttcct
catctgtaaaatggg

The reverse-complement of the Gilbert & Labuda 65-bp CORE-SINE sequence is:

[SEQ ID NO: 8]

cccattttacagatgaggaaactgaggctcagagaggttaartracttgc
ccaaggtcacacagc

Interestingly, the inverted MIR element of the MAPT-AS1 DNA sequence contains two 7-mer motifs that are either complementary or identical to two conserved regions of the human 18S rRNA. In accordance with the underlined motifs in the above RNA sequences (SEQ ID NOs: 1-3), these motifs are CACCCAC and CTGAGGC in the MAPT-AS1 DNA sequence. CACCCAC is complementary to nucleotides 1318-1324 of the 18S rRNA at the basis of helix 33, and CTGAGGC is identical to nucleotides 905-911 in the stem 21esd6, which is part of the 18S rRNA expansion segment 6, only present in Eukaryotes. It is thought that both of these MIR motifs could mediate MAPT IRES repression due to a direct competition for pairing with the 40S rRNA (FIG. 18).

It is envisaged that any MIR domain can be used in the therapeutic RNA of the invention. Preferably, the MIR domain used in the invention comprises one or both of the ‘CACCCAC’ and ‘CUGAGGC’ motifs. (In light of the conservation of these sequences between humans, non-human primates and mice; in some embodiments of the present invention, the CTGAGGC DNA motif (CUGAGGC RNA motif) may be taken as including the adjacent adenine residues to form an AACTGAGGC DNA motif (AACUGAGGC RNA motif), which may be present as AACUGAGGC in the therapeutic RNA of the invention.) Where the therapeutic RNA of the invention comprises both of the ‘CACCCAC’ and ‘CUGAGGC’ motifs, these motifs will typically be separated by about 17 nucleotides, e.g. by about 9-25 nucleotides, by about 10-24 nucleotides, by about 11-23 nucleotides, by about 12-22 nucleotides, by about 13-21 nucleotides, by about 14-20 nucleotides, by about 15-19 nucleotides, by about 16-18 nucleotides, or by 17 nucleotides.

The inventors' kmer-enrichment analysis on all antisense MIR-lncRNAs revealed that MIR-lncRNAs target genes are enriched for 7-mer motifs in their 5′-UTRs, matching the 18S rRNA “active region” as defined by Weingarten-Gabbay et al.²⁵ (Table 1). Surprisingly, 135 (27.7%) out of 487 human genes overlapping within their 5′-UTR with antisense MIR-lncRNAs, possess one or more motifs (7-mers) of 18S complementarity which are also found in the MIR element of their paired MIR-lncRNA. Strikingly these motifs cluster in specific areas within the 18S rRNA “active region” (FIG. 17, Table 1).

### Target Genes

The target gene, the expression of which is capable of being modulated by the therapeutic RNA of the invention, may be selected from Table 1. For example, the target gene may be MAPT, SNCA, APP, MBNL1, SLC1A2, TPP1, DHCR24, ECE1, IMMT, FADD, MATR3, CDKN2A, DDX20, UCHL1, PRPH, GARS, DCTN1, ZNF224, KLK6, BDNF, PPP3CB, CELF1 or DERL1.

### BRIEF DESCRIPTION OF THE FIGURES

FIG. 1—MAPT-AS1 is a brain-enriched antisense lncRNA expressed during neuronal differentiation

a, human MAPT-AS1 and MAPT genomic region (hg19).

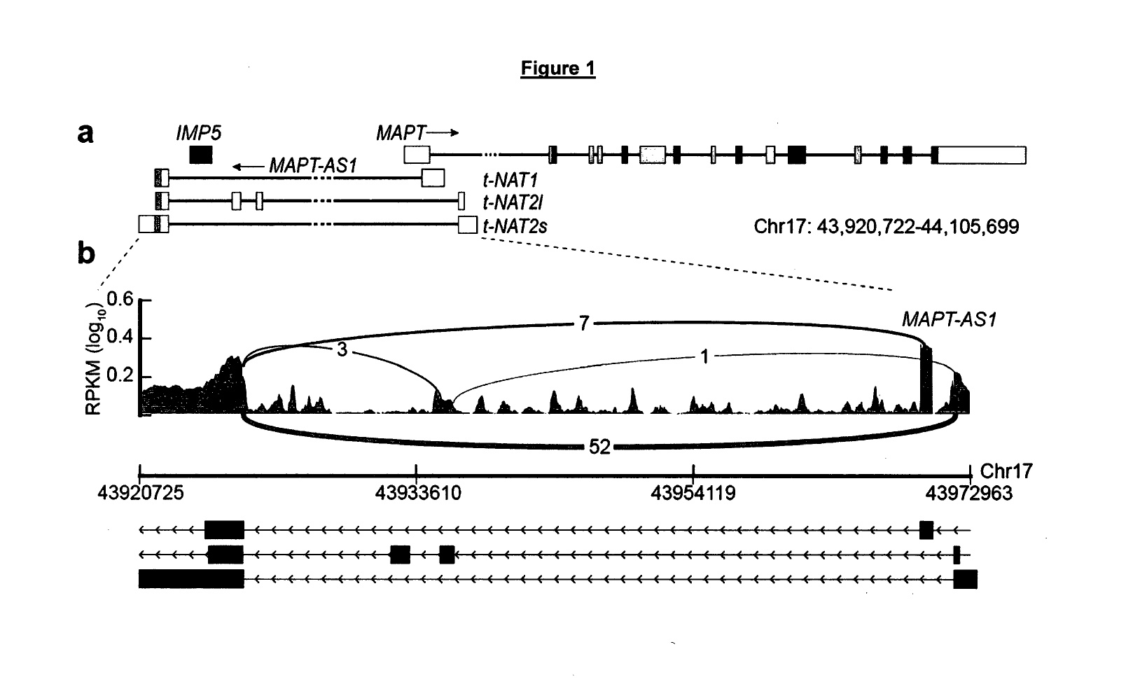

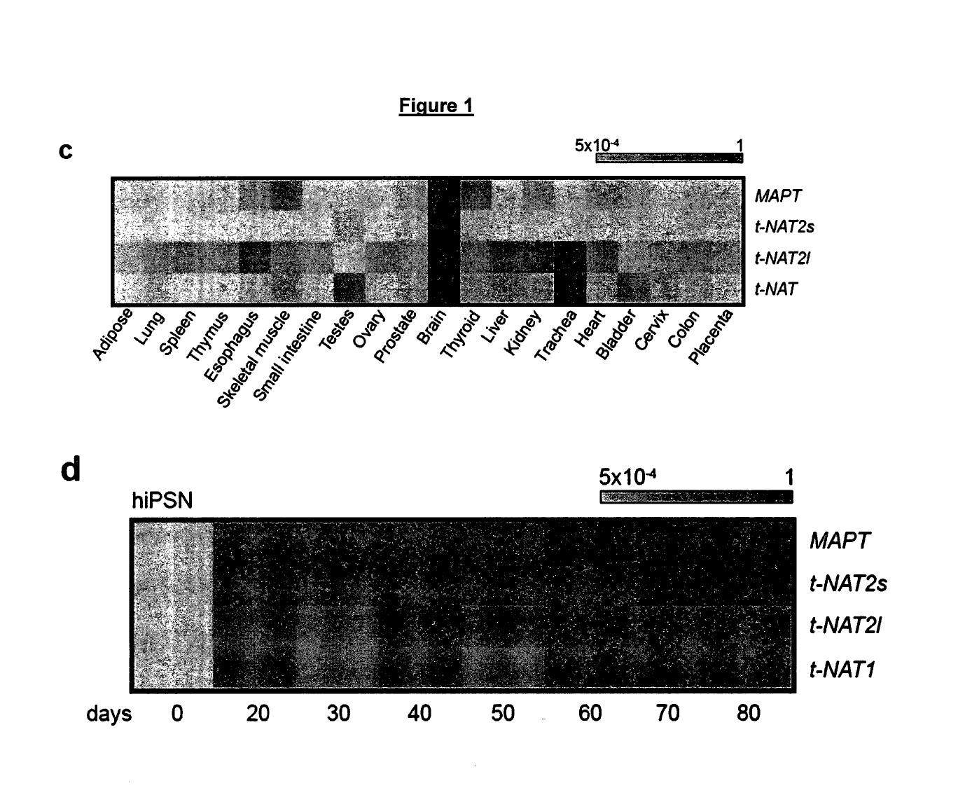

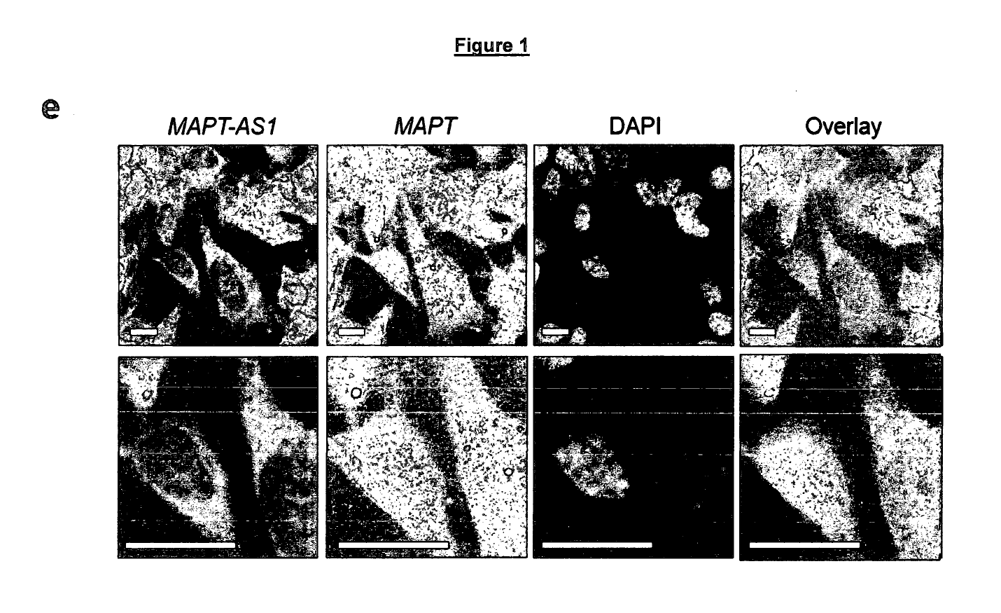

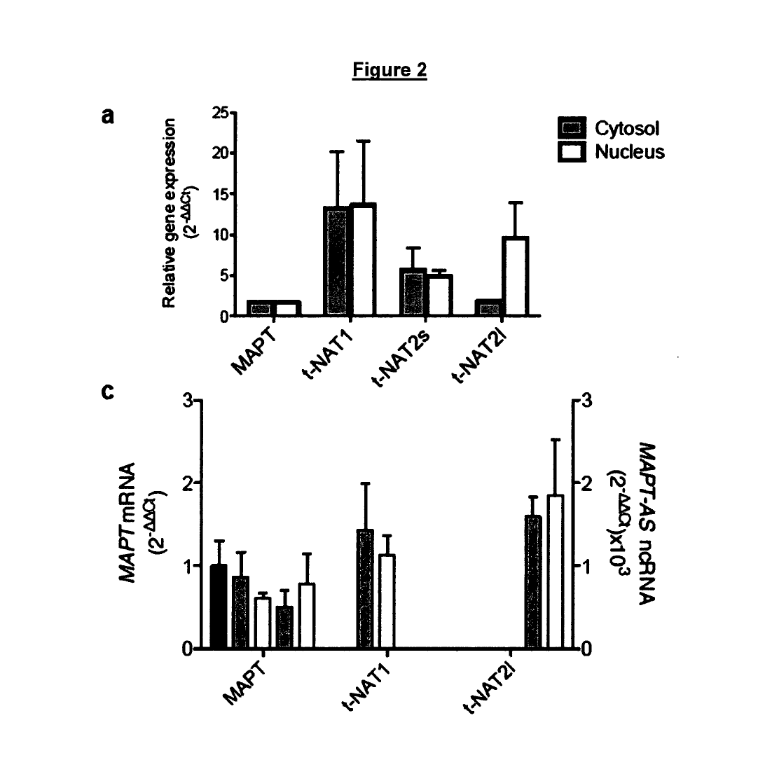

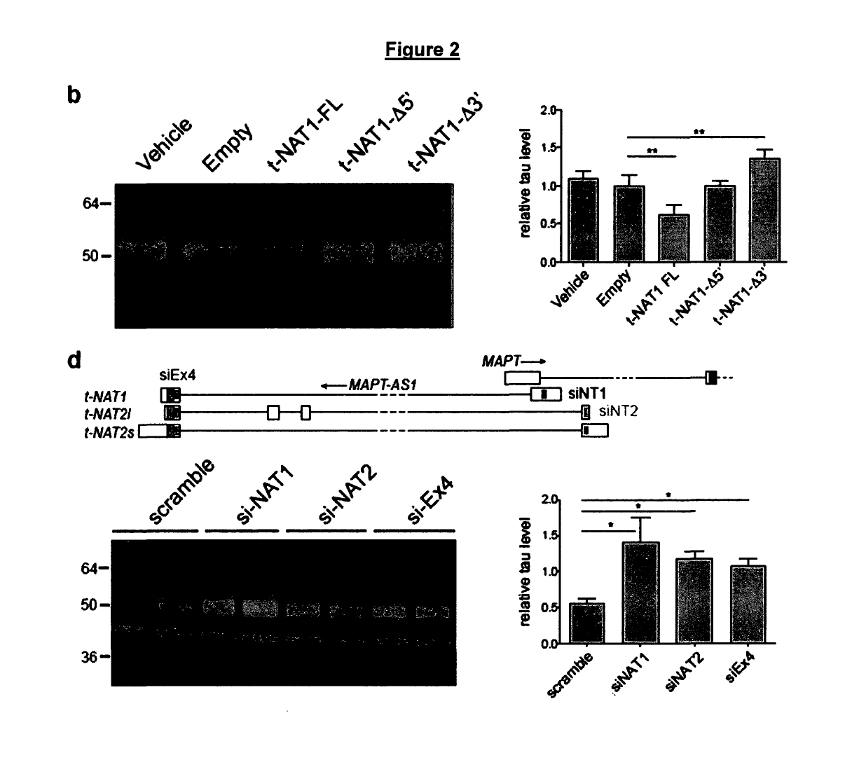

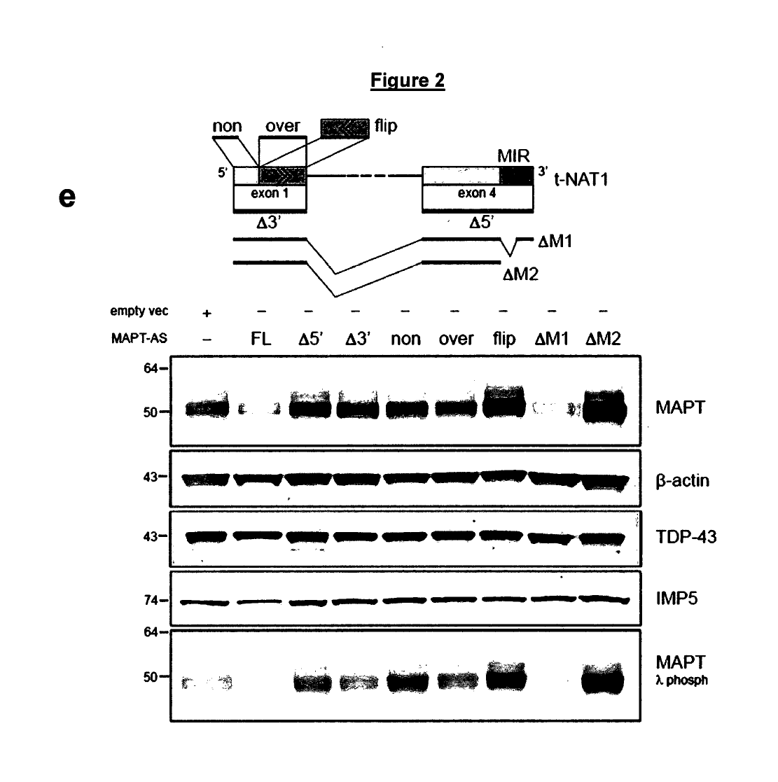

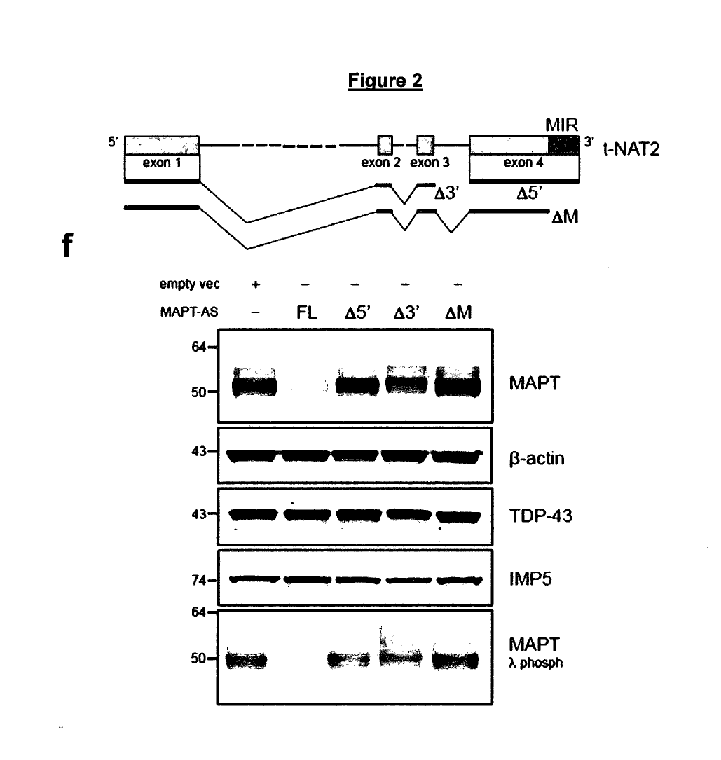

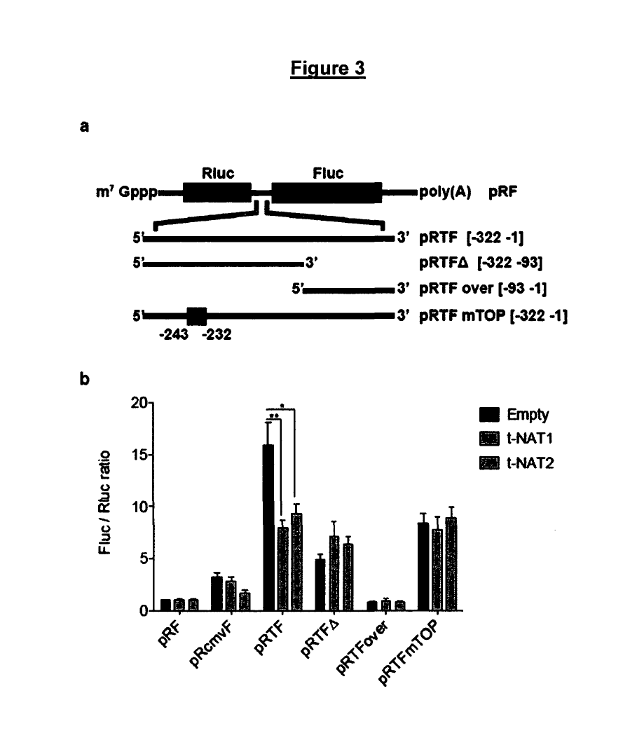

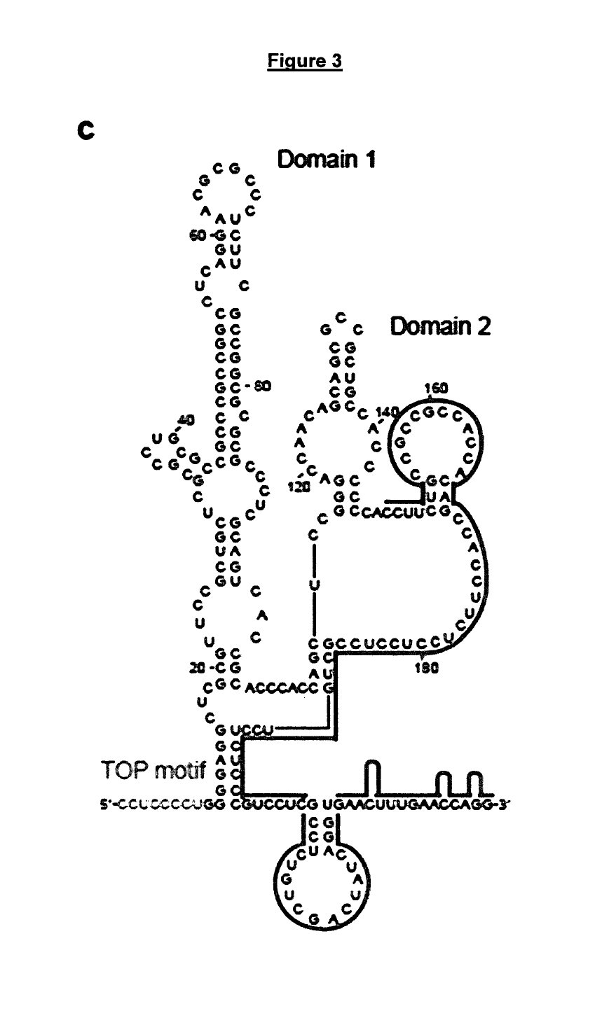

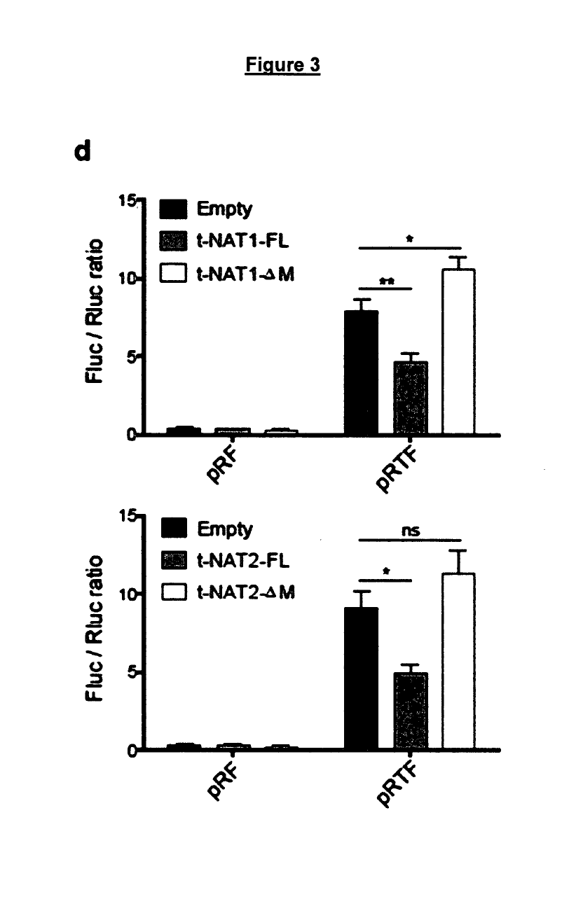

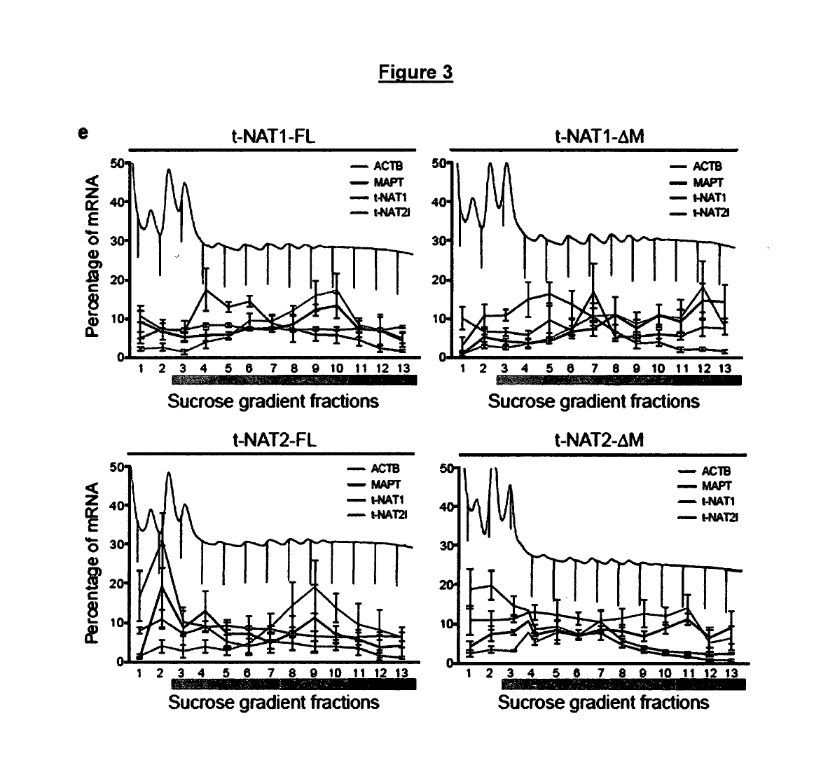

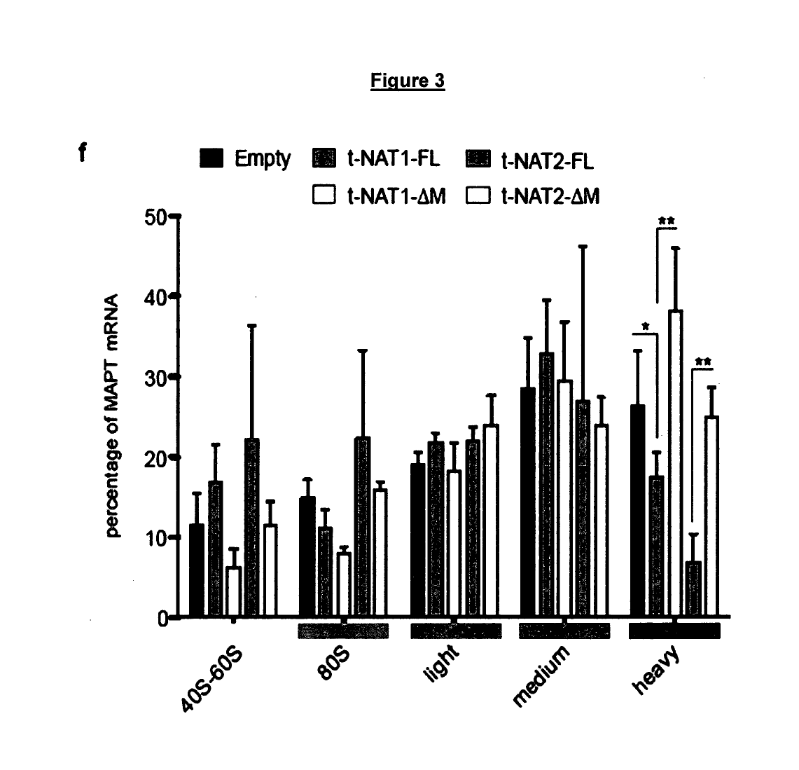

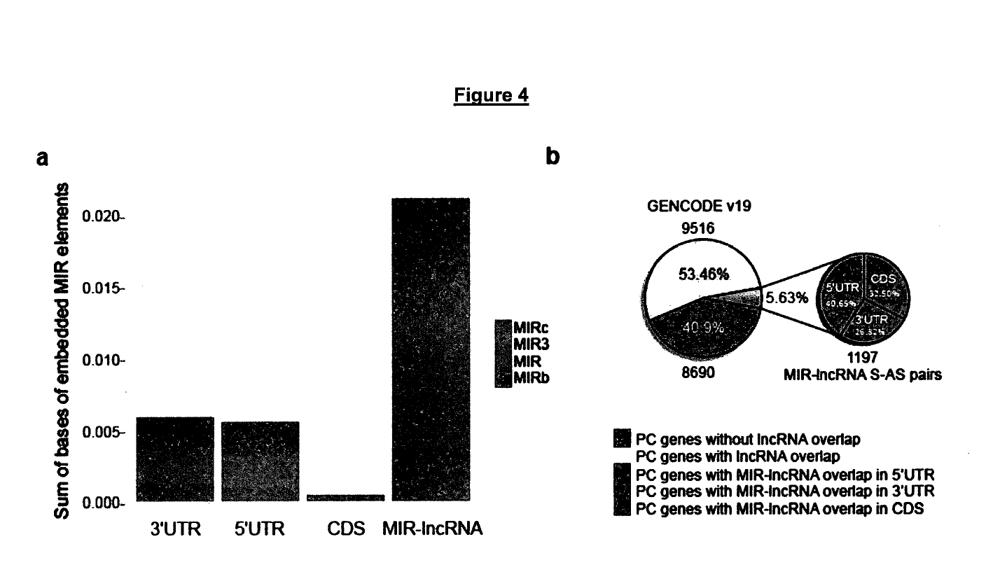

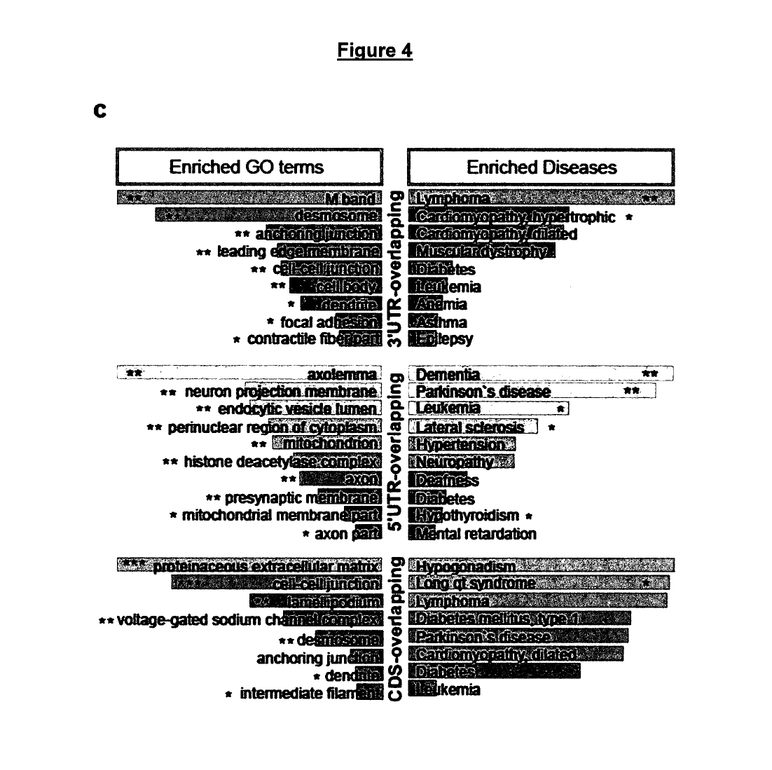

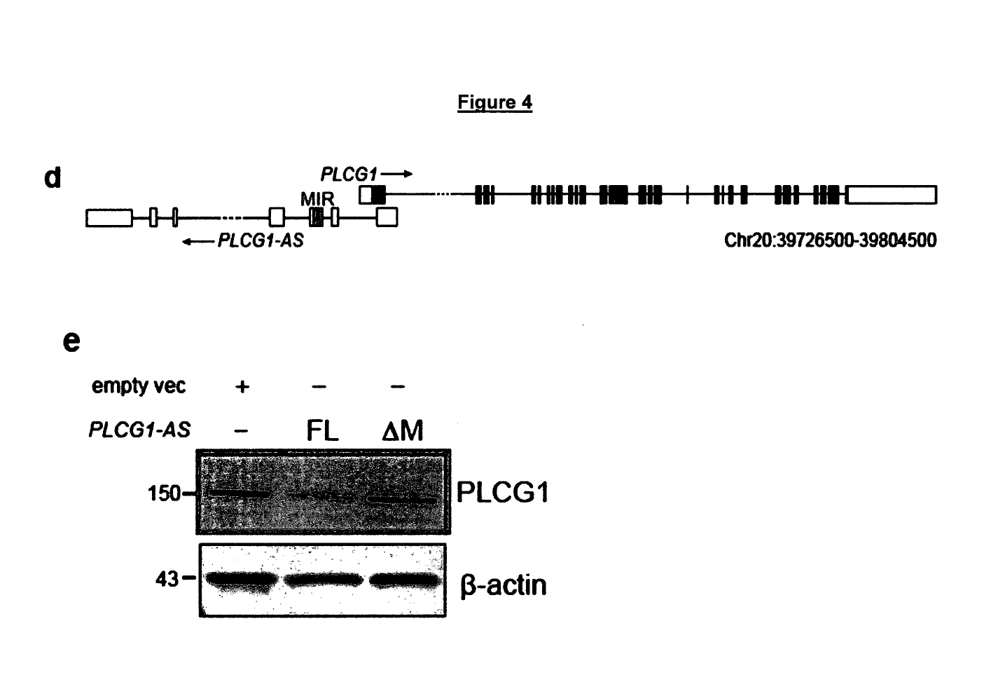

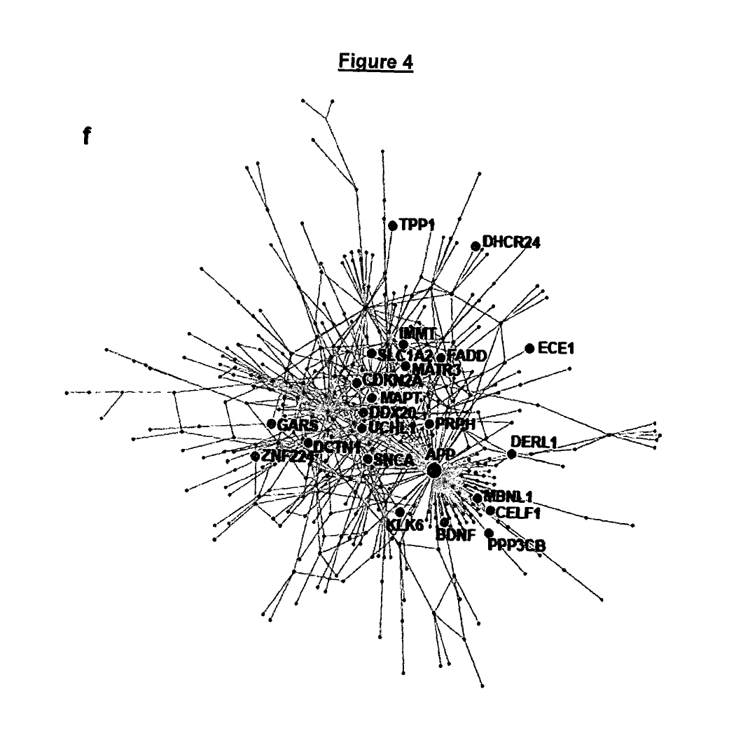

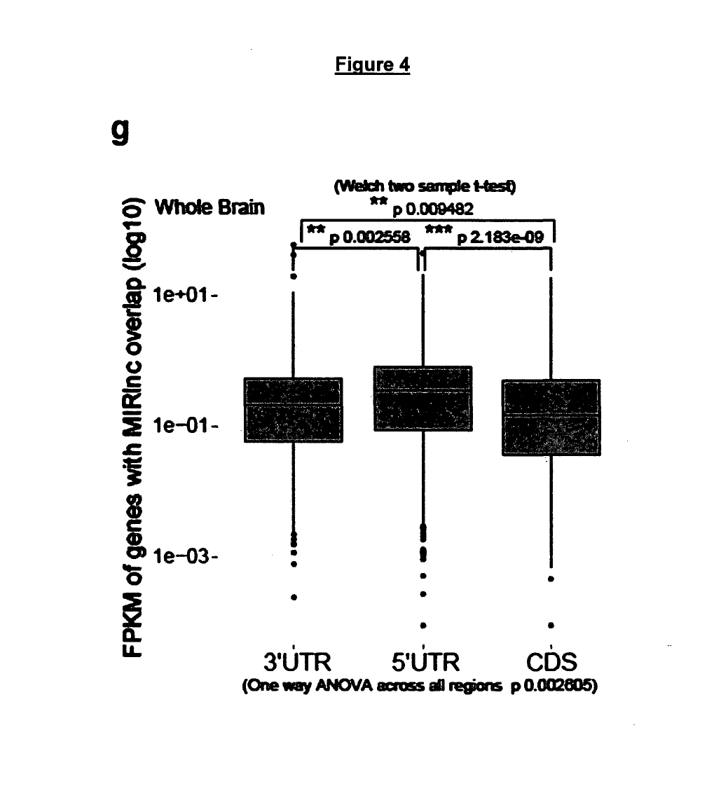

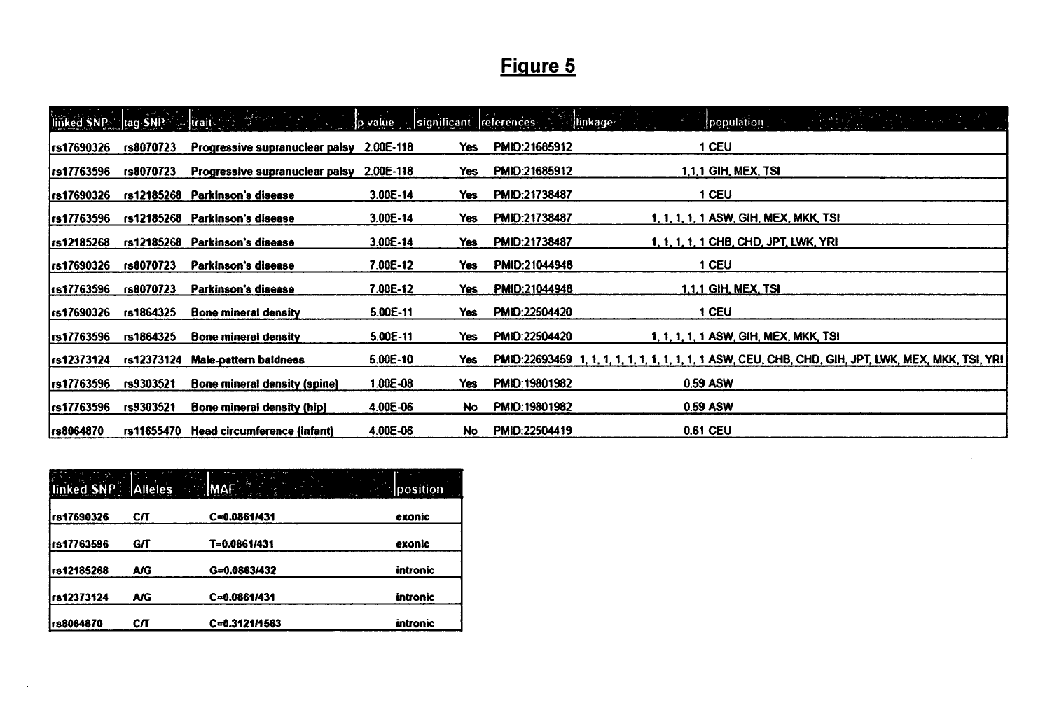

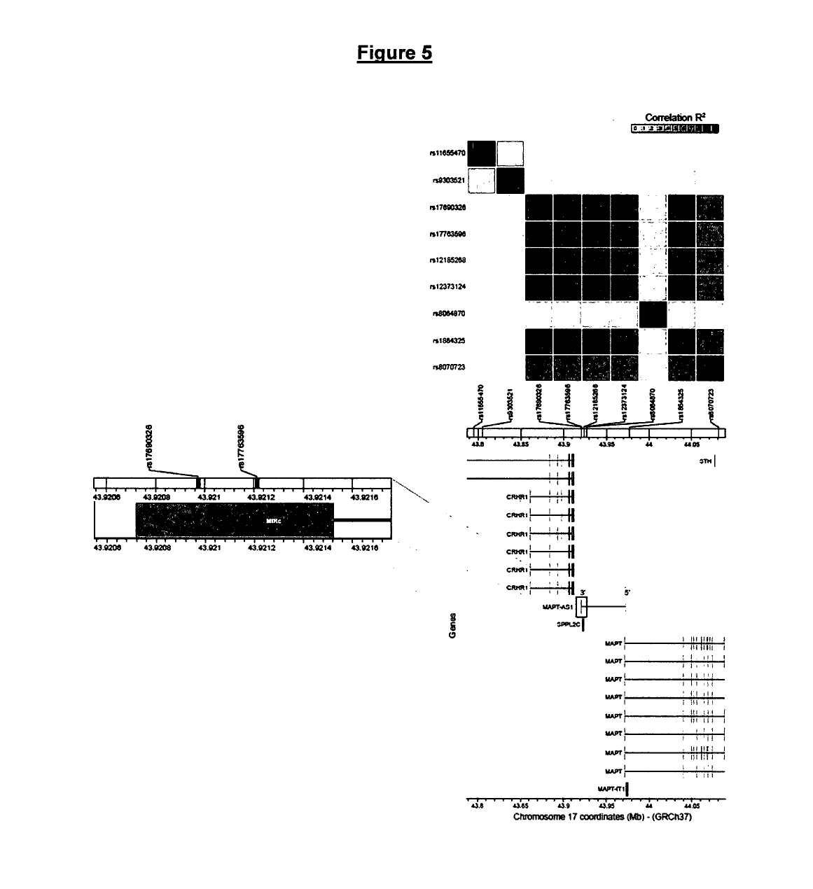

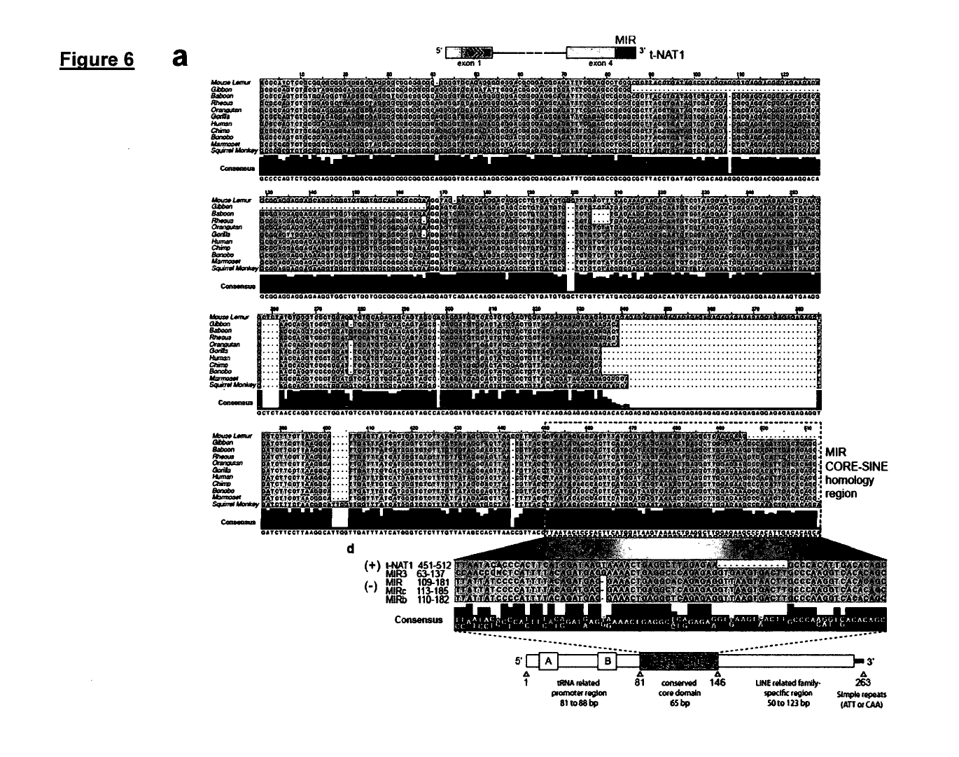

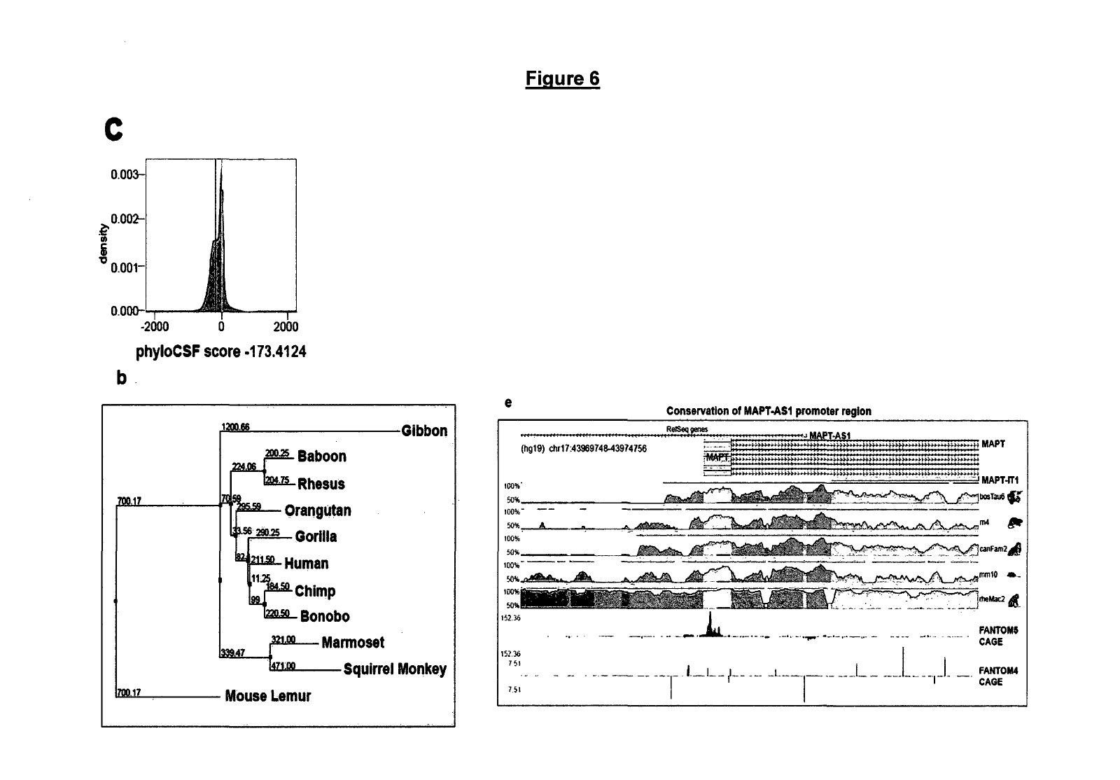

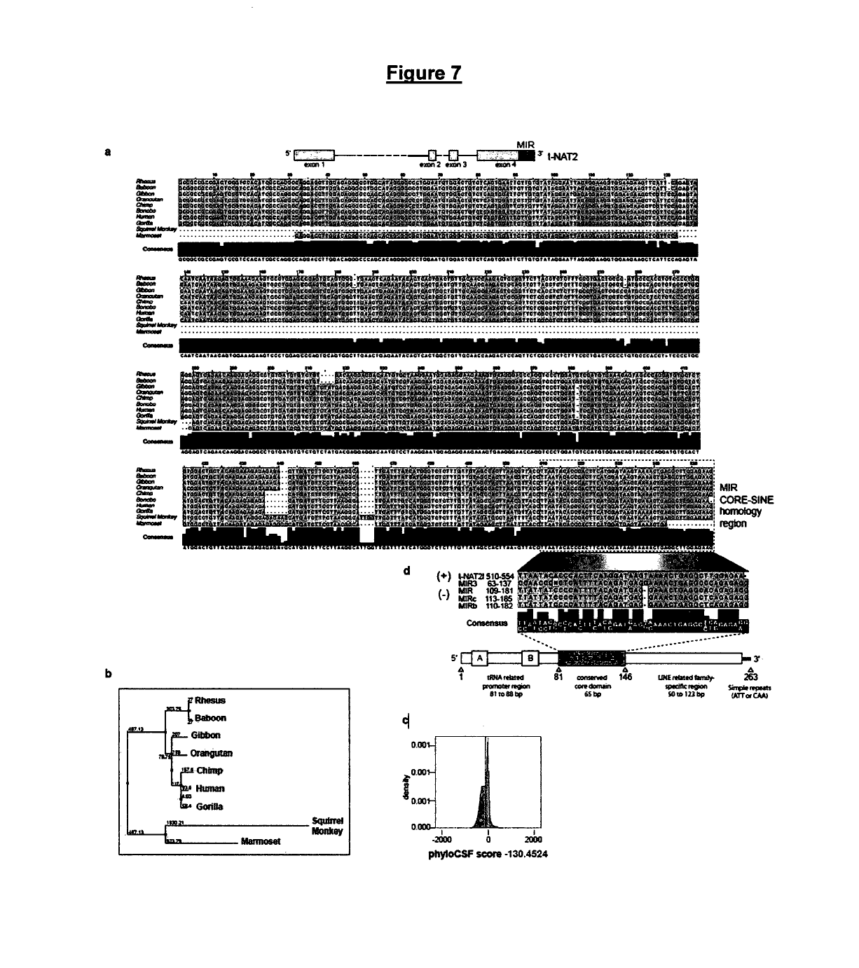

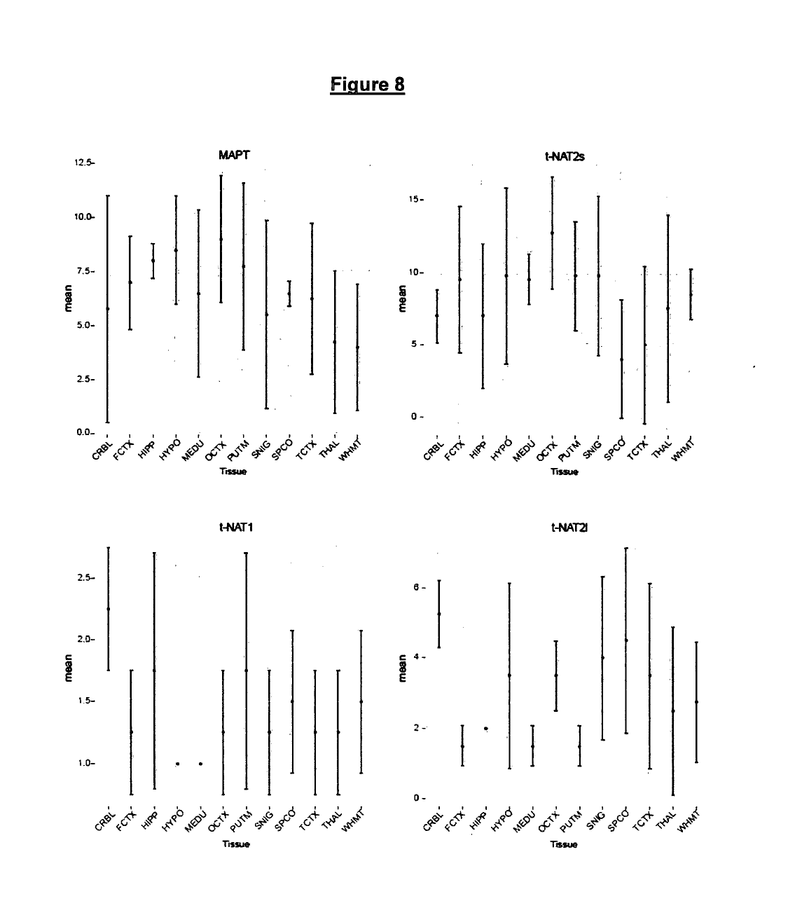

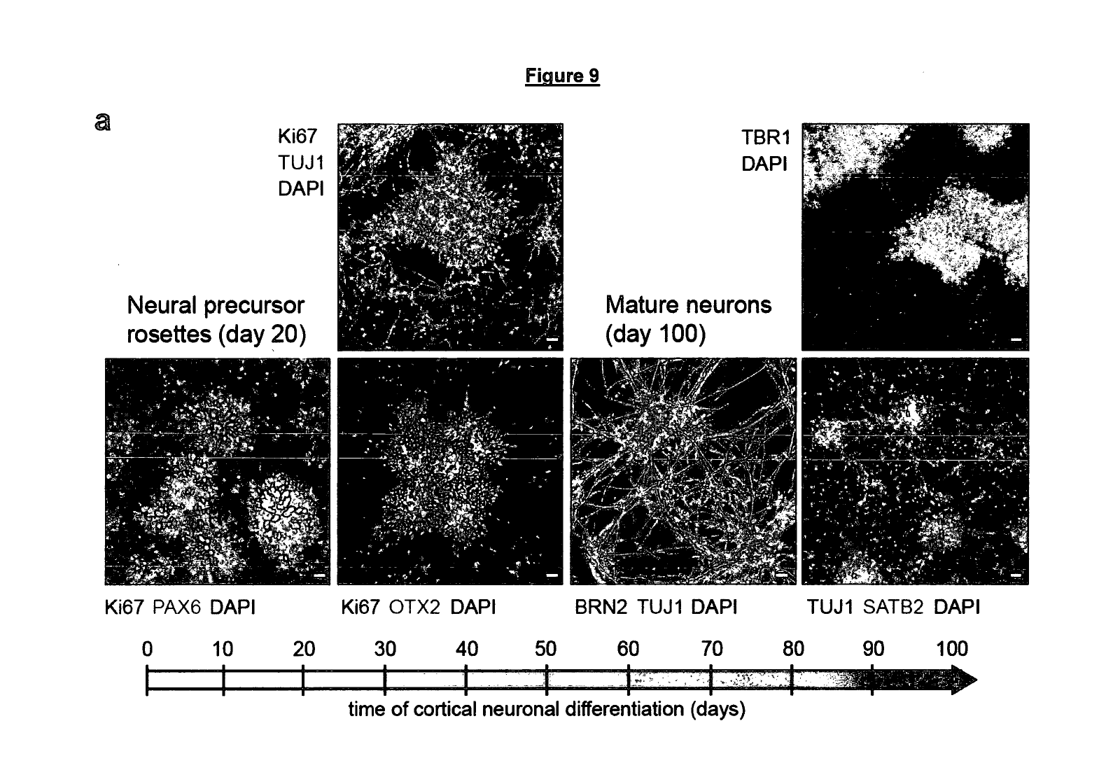

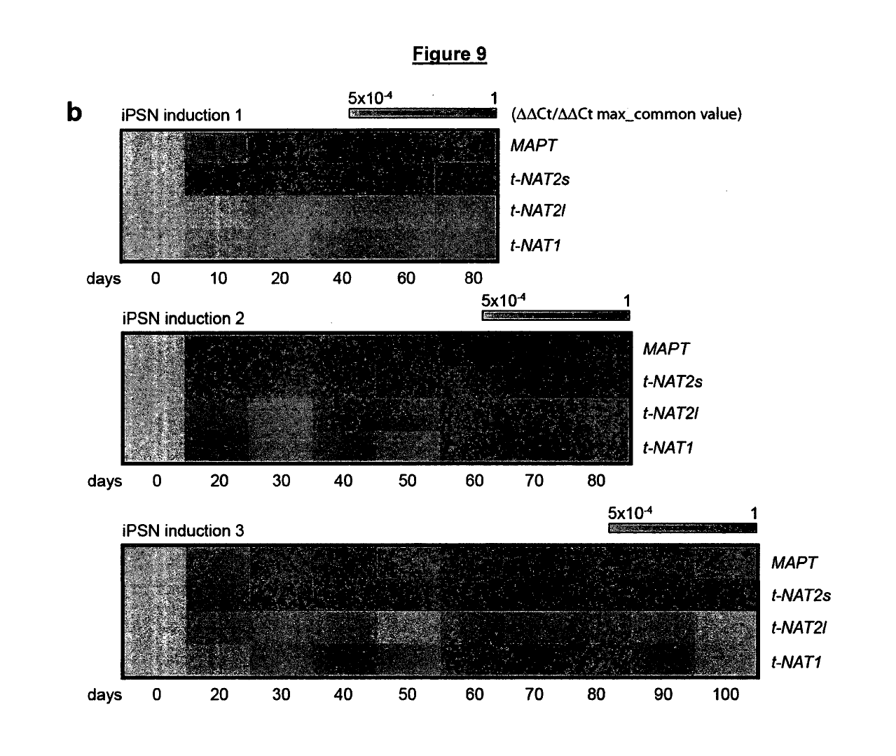

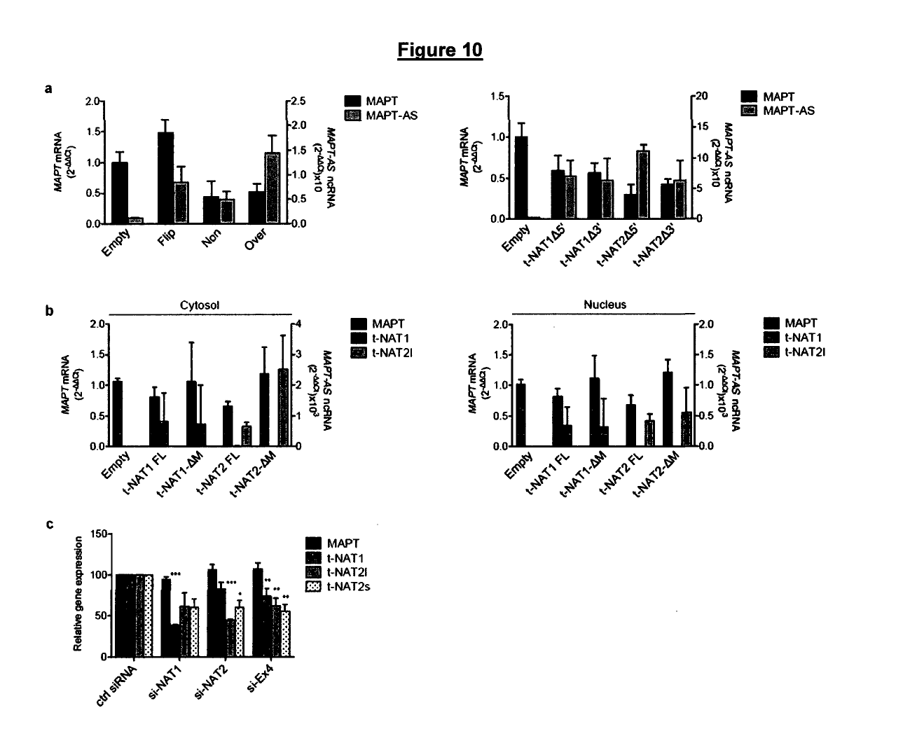

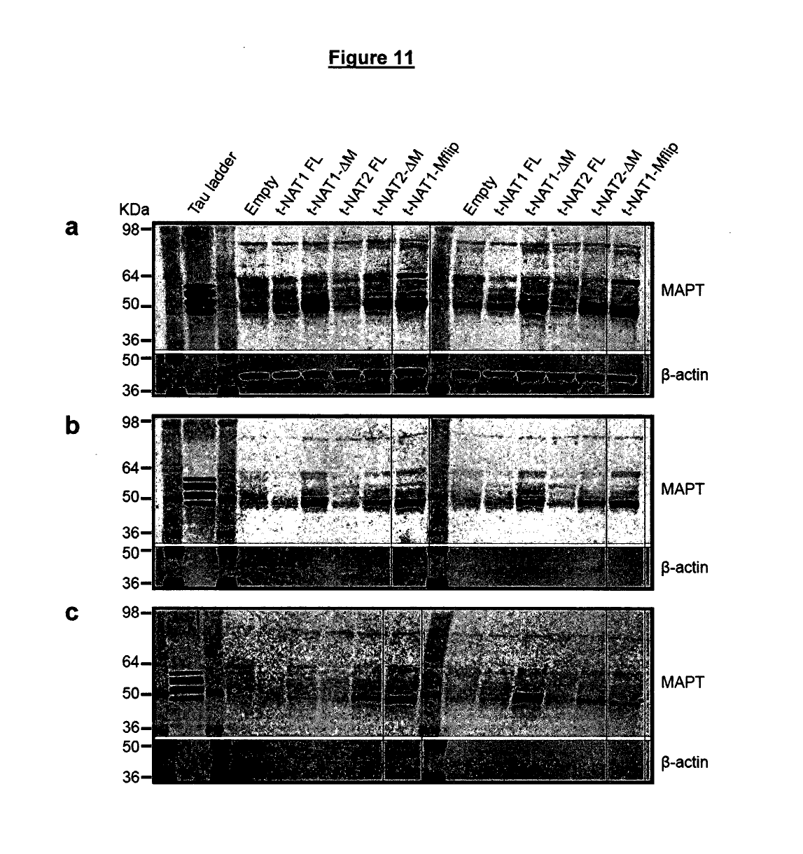

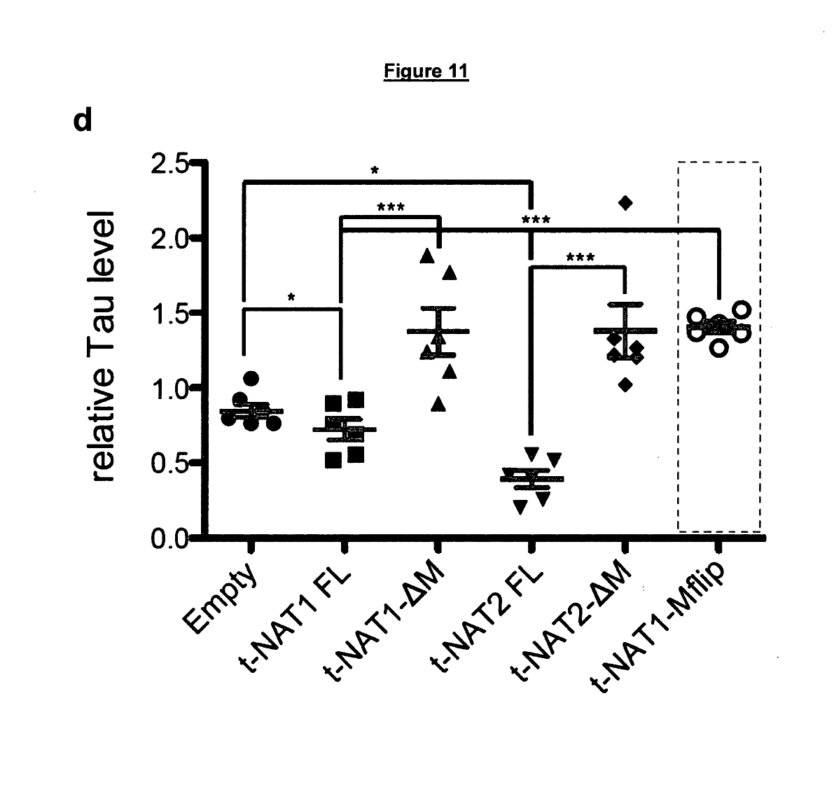

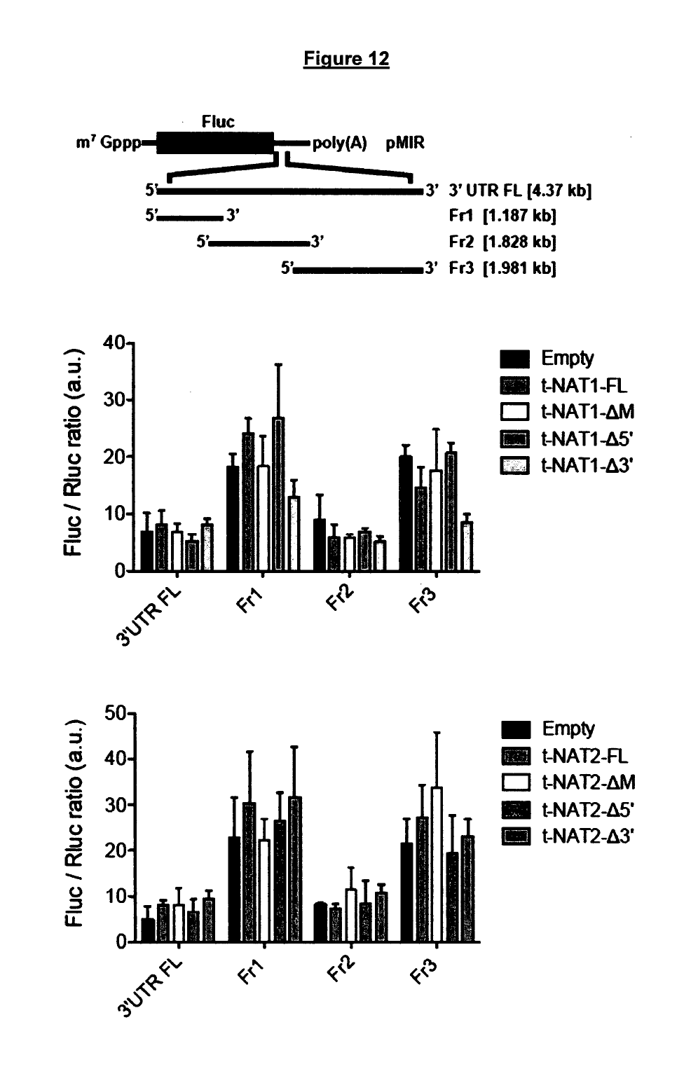

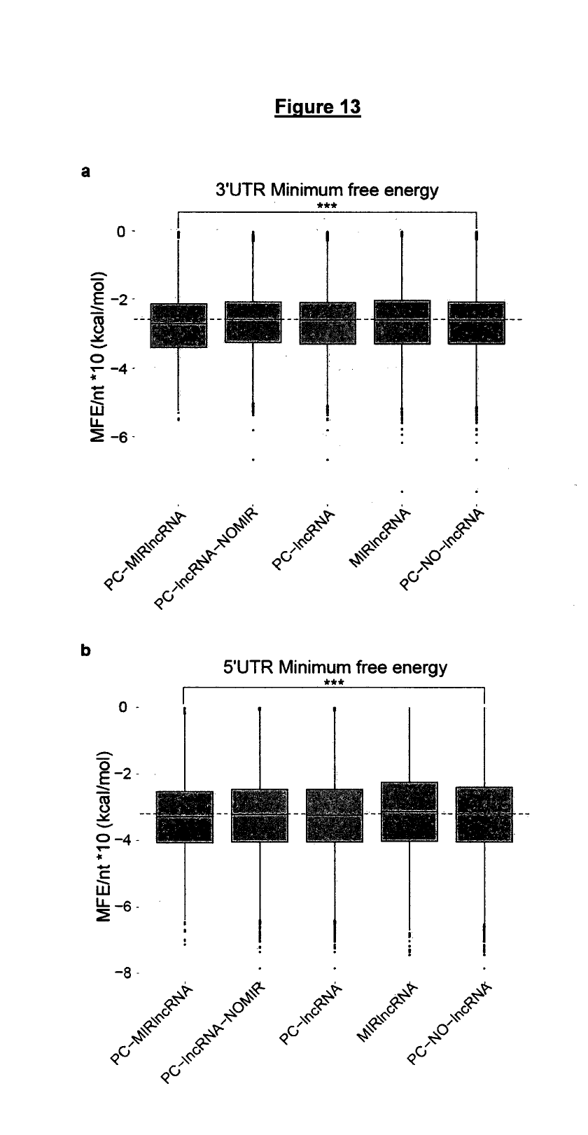

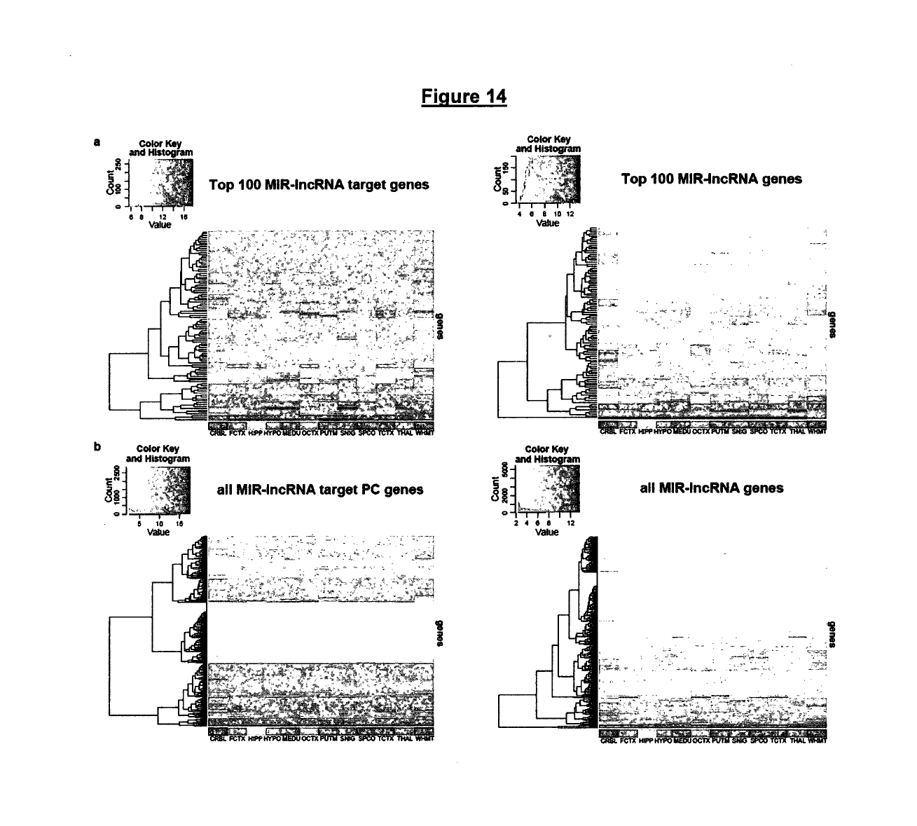

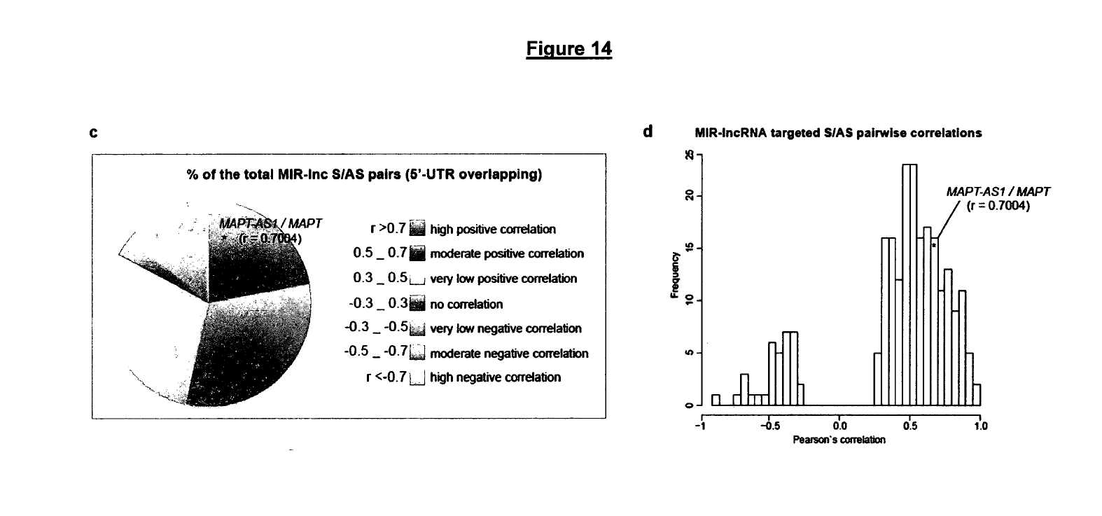

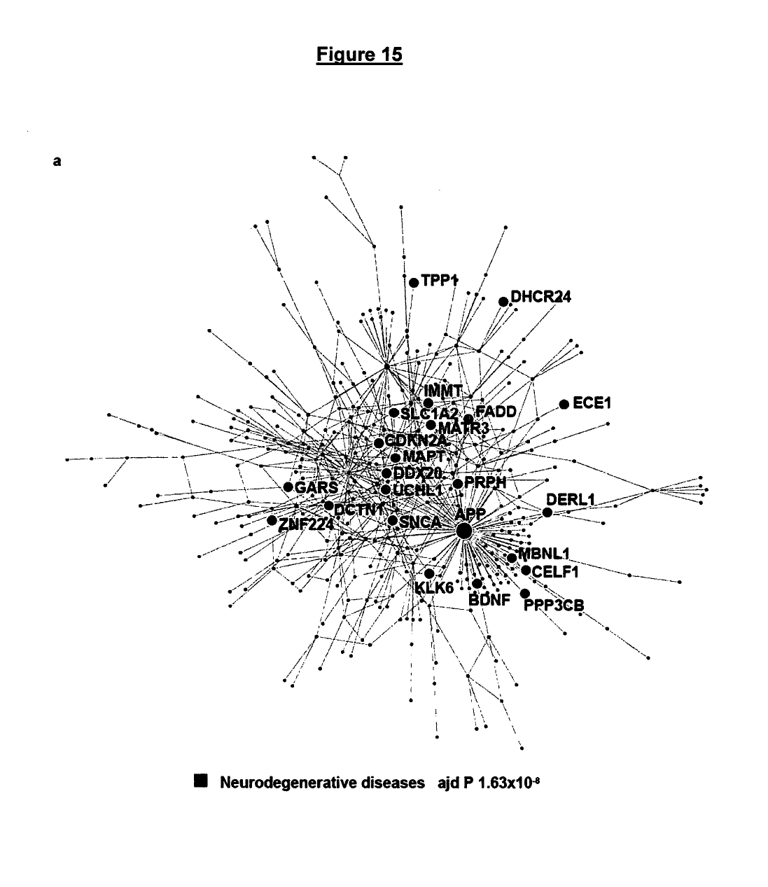

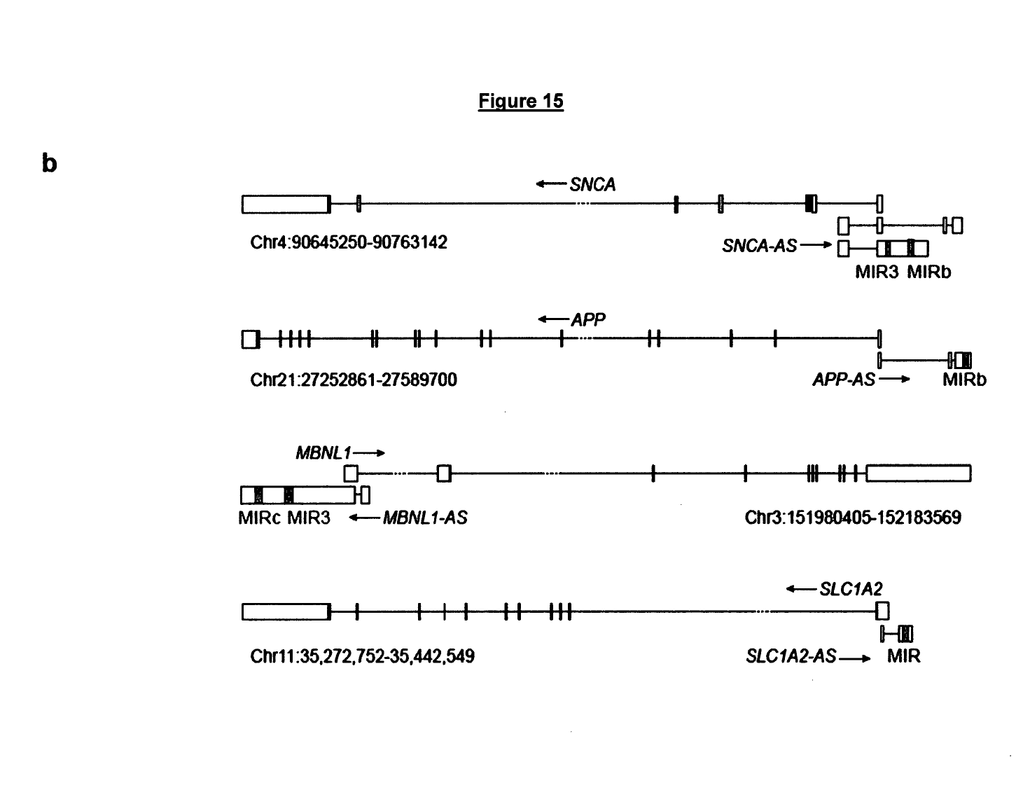

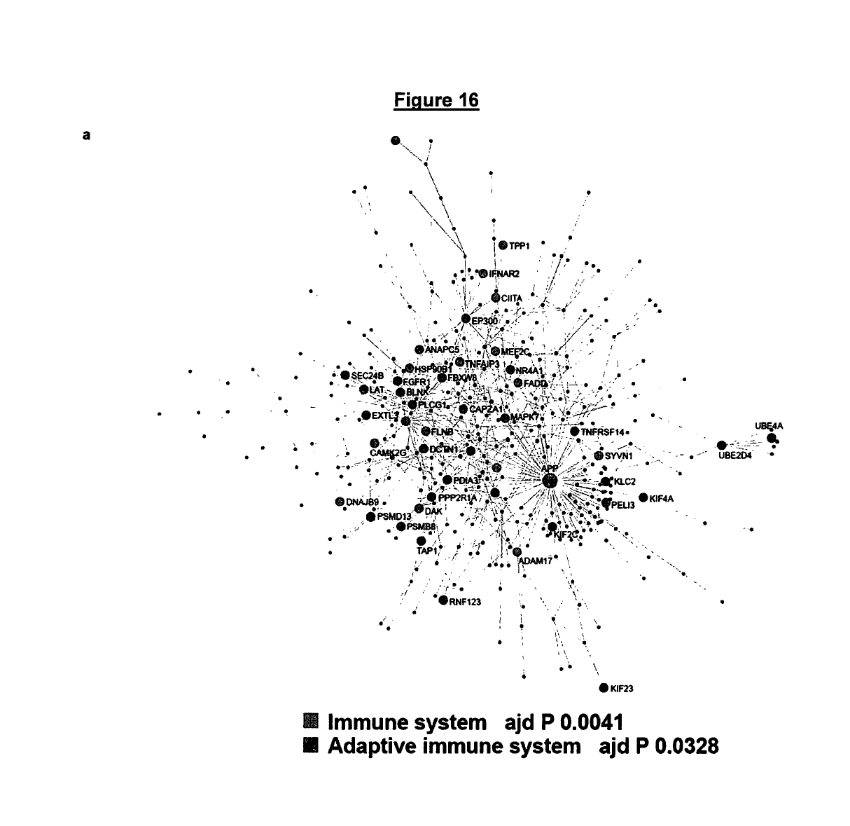

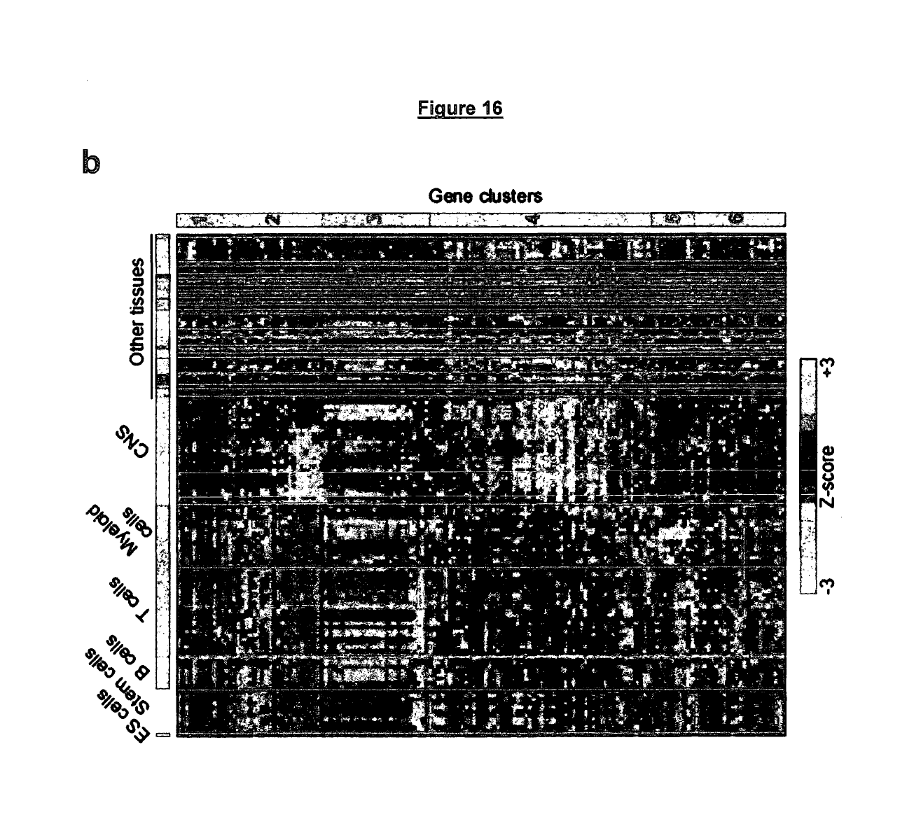

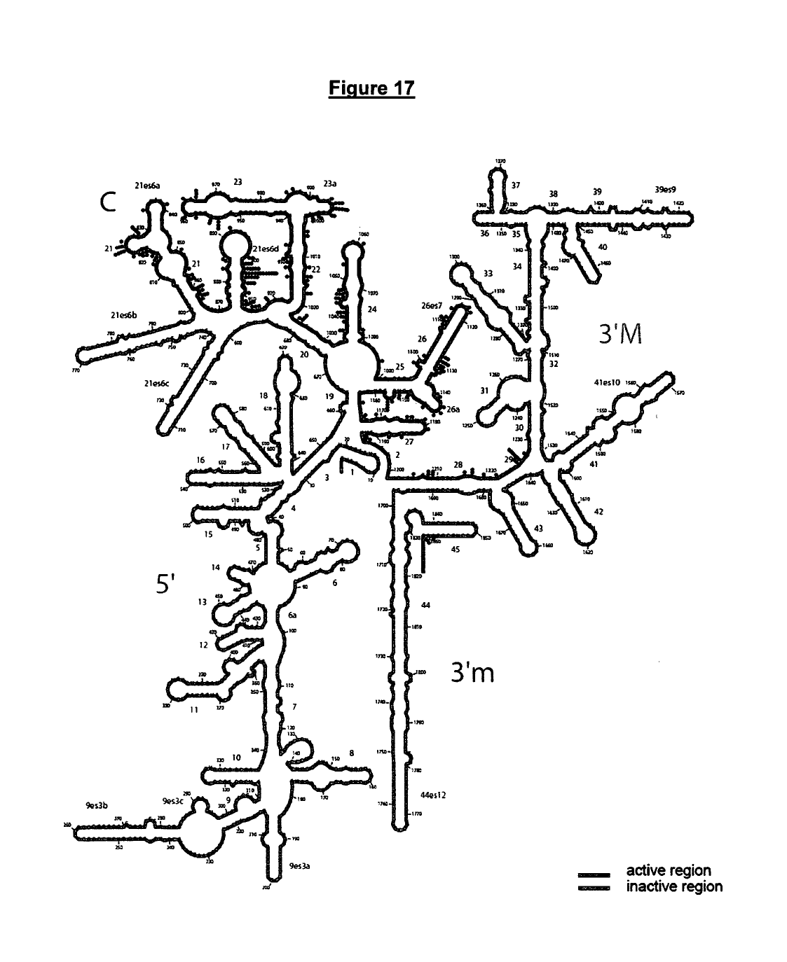

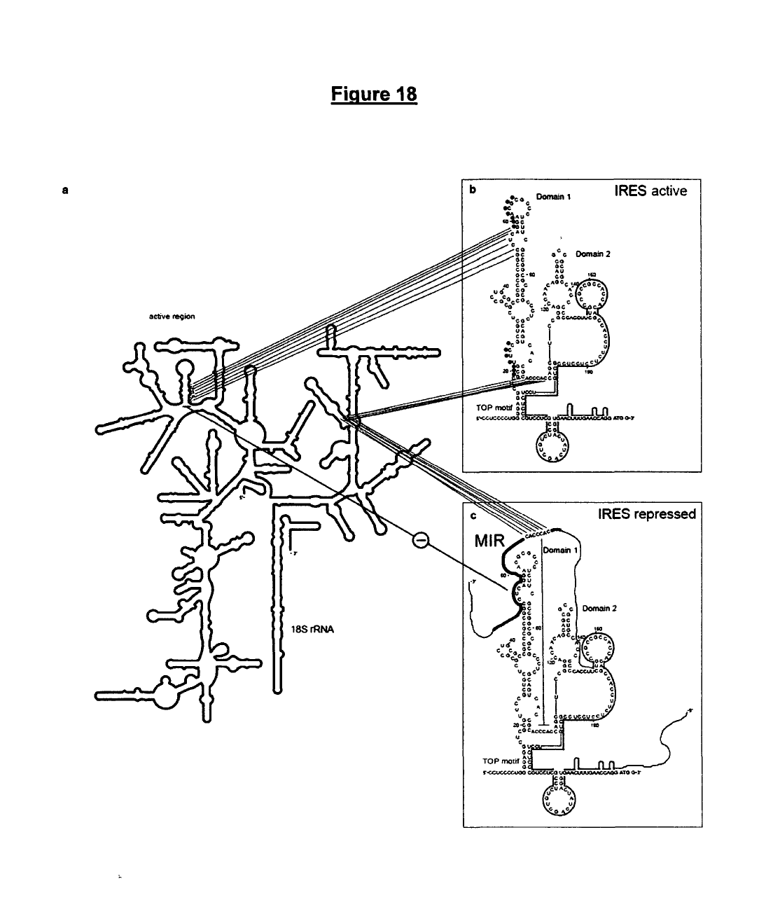

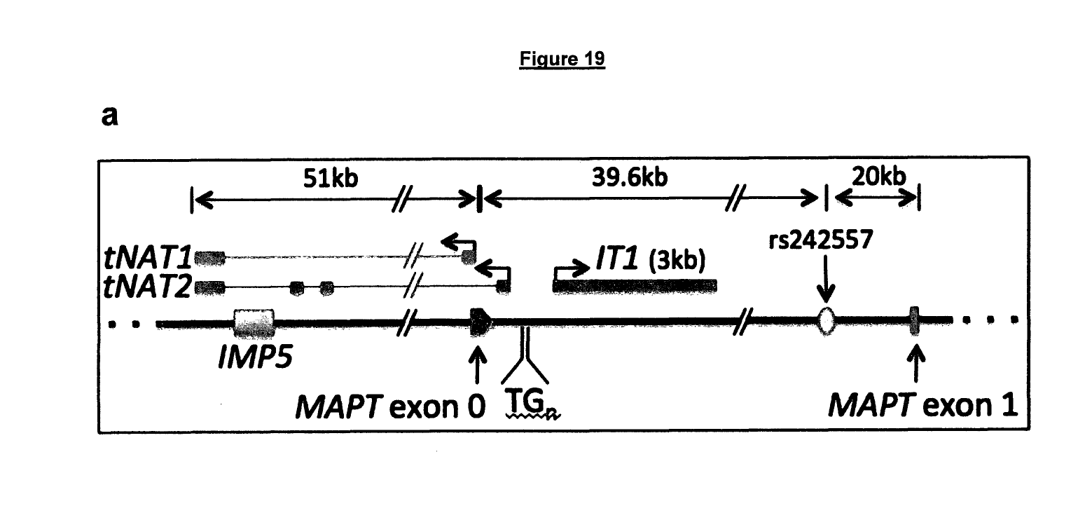

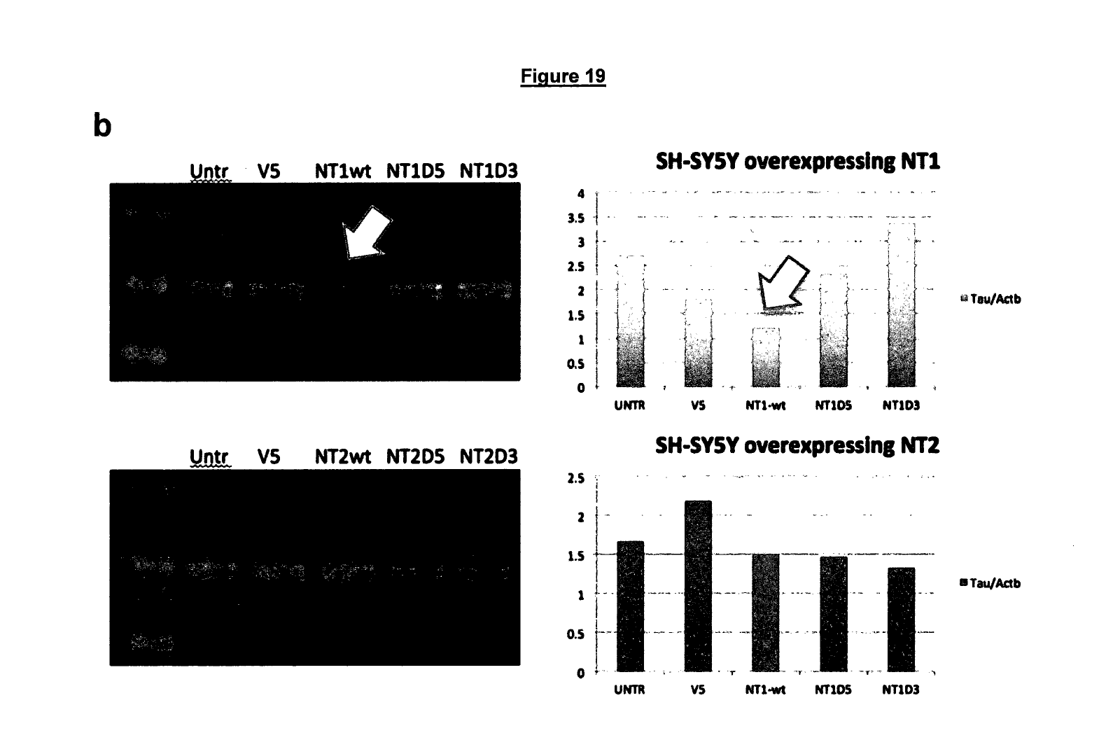

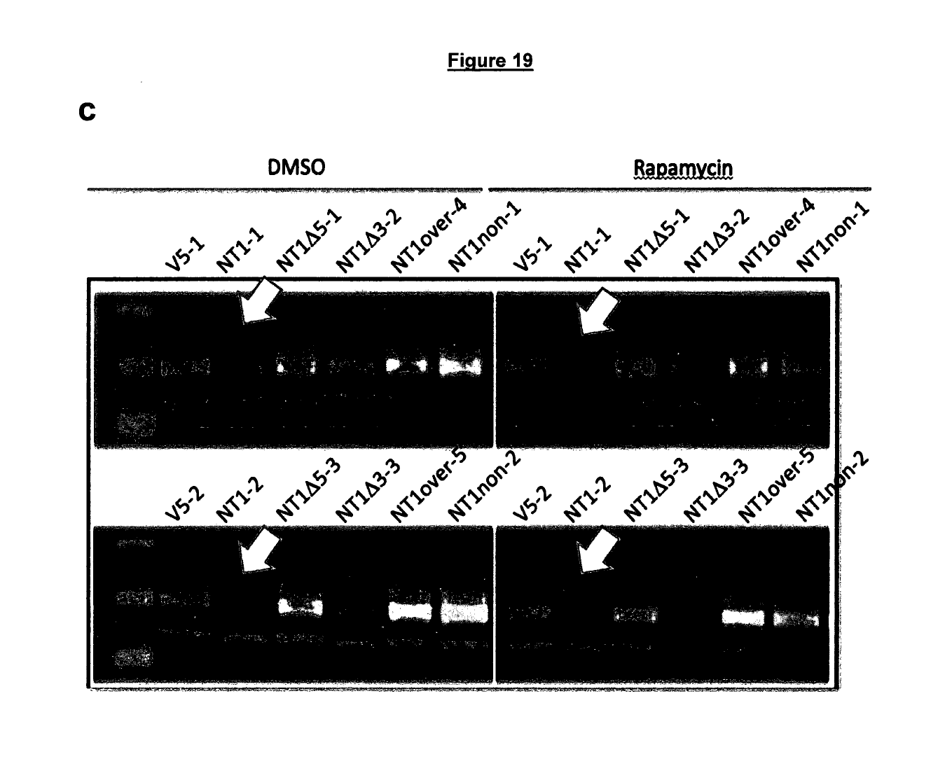

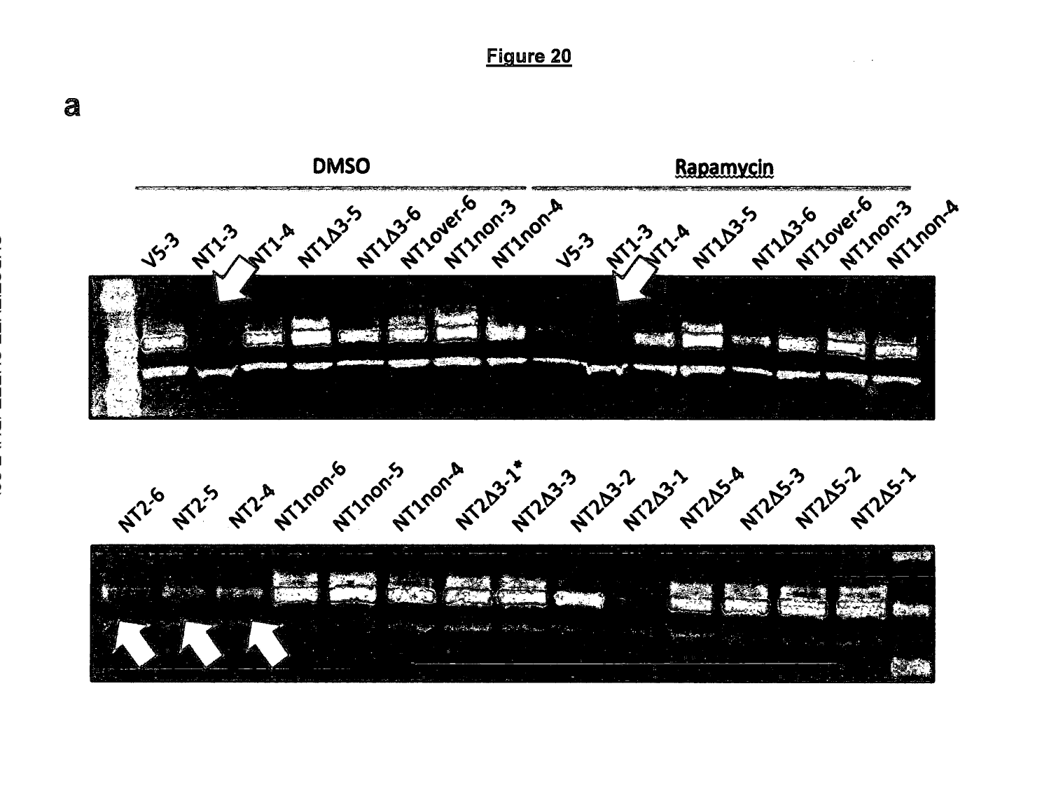

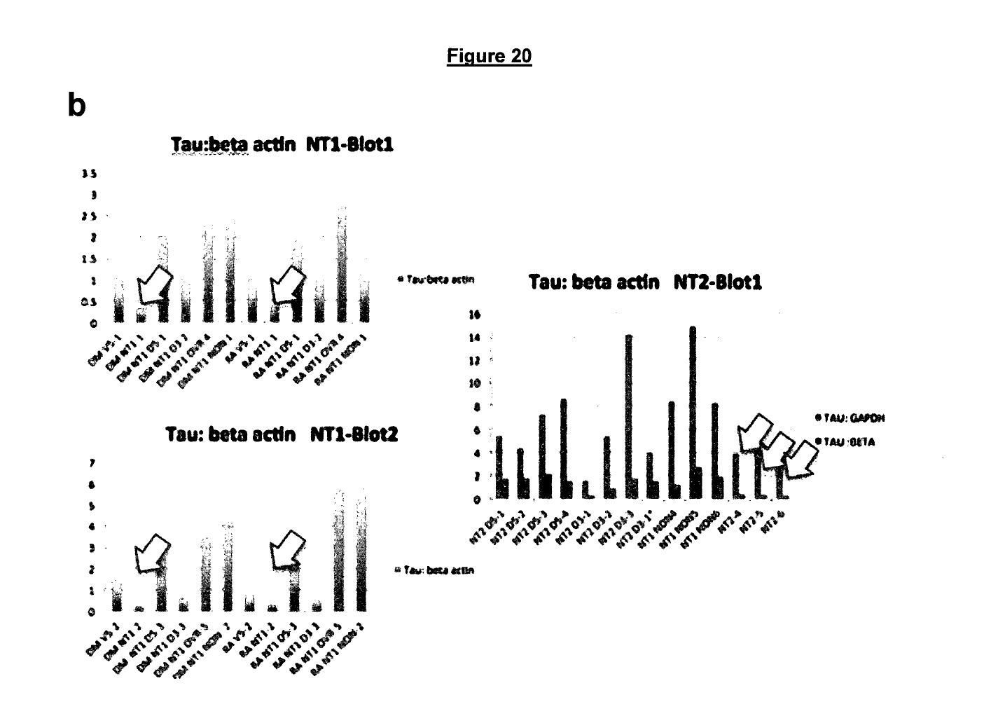

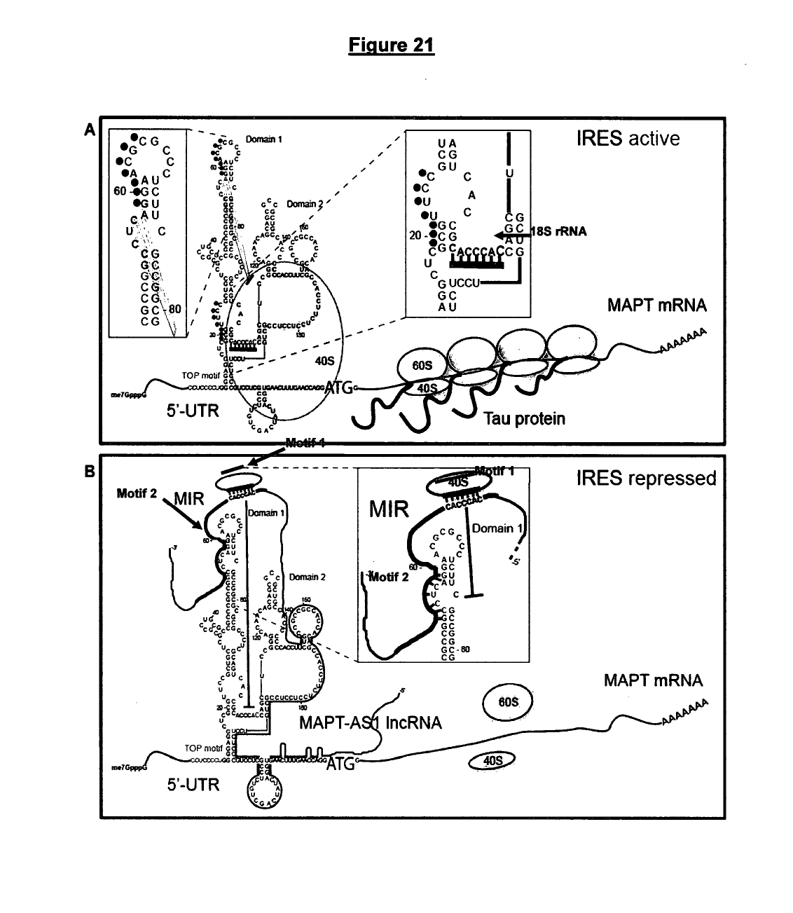

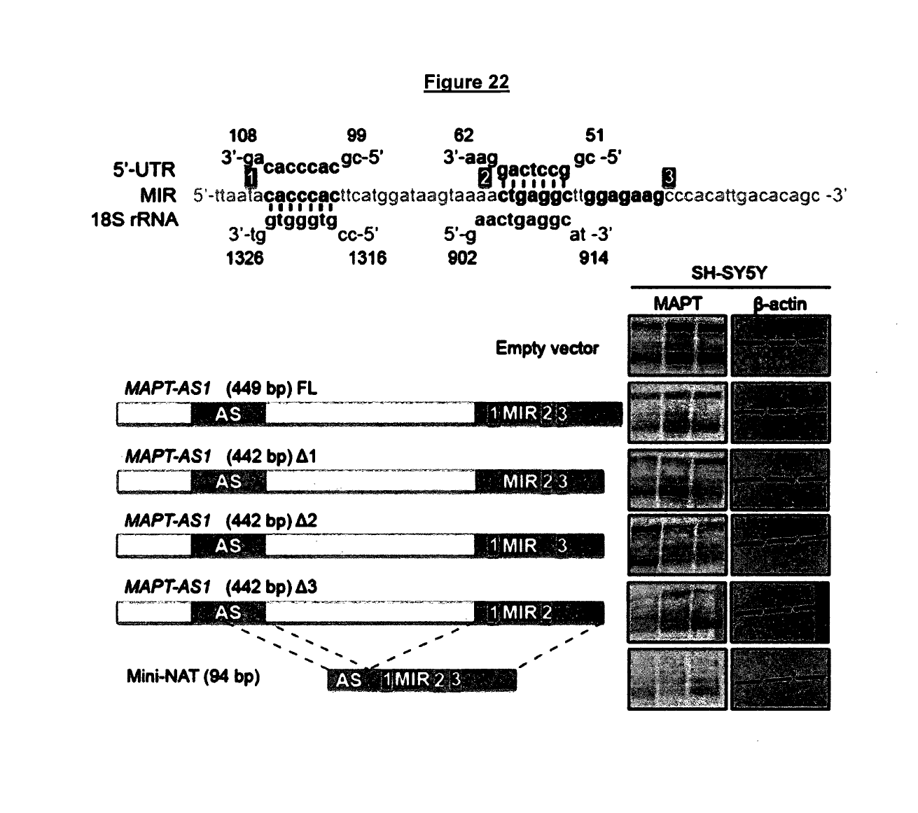

MAPT constitutive exons are in black; alternatively spliced exons are in grey; 3′ and 5′ UTRs are in white; antisense MAPT exons are in white; repetitive elements are in red (MIR), which are seen as the small rectangles within the exons at the left-hand side of FIG. 1a. Introns are represented as lines.

b, Sashimi plot of RNA-Seq peaks from human brain (log₁₀RPKM); numbers over connecting lines represent counts associated to each splice junction.

c, Quantitative expression of MAPT and MAPT-AS1 in twenty human tissues by qRT-PCR (2^{-ΔΔCt}/2^{-ΔΔCt}ₘₐₓ) d, Quantitative expression of MAPT and MAPT-AS1 in human iPSC differentiated into cortical neurons (from 0 to 80 days in culture) measured by qRT-PCR (ΔΔCt/ΔΔCtₘₐₓ).

e, MAPT-AS1 (green) and MAPT (grey) transcripts are expressed in the nucleus stained by DAPI (blue) and cytoplasm of SH-SY5Y neuroblastoma cells. Scale bars represent 10 μm.

FIG. 2—MAPT-AS1 controls MAPT translation through an embedded inverted MIR element

a, Quantitative expression of human MAPT-AS1 and MAPT transcripts as measured by qRT-PCR (2^{-ΔΔCt}) in SH-SY5Y cells after cellular fractionation.

b, Full-length MAPT-AS1-transfected SH-SY5Y cells (t-NAT1-FL) show decreased levels of endogenous tau protein (green) normalized to β-actin (red). Cells transfected with MAPT-AS1 deleted of the 5′exon (t-NAT1-Δ5′) do not show any significant change of endogenous tau levels, whereas deletion of the 3′-exon (t-NAT1-Δ3′) is associated with a significant increase in endogenous tau level. Data in a and b indicate mean±s.d., n≥3.

c, Quantitative expression of human MAPT-AS1 and MAPT transcripts as measured by qRT-PCR (2^{-ΔΔCt}) in independent clones stably expressing each type of construct (empty vector, t-NAT1 full-length, t-NAT21 full-length).

d, siRNAs targeting three different exons of MAPT-AS1, as shown in the scheme, cause an increase of endogenous tau protein in SH-SY5Y cells (mean±s.d., n≥3)

e, f, Full-length (FL) t-NAT1 and t-NAT21 are required for regulating endogenous tau (cells stably expressing t-NAT1, left panel and cells stably expressing t-NAT21, right panel). e, f, Inverted MIR is sufficient to control endogenous tau protein levels in stably expressing SH-SY5Y cells. Scheme of mutants is shown in 5′ to 3′ orientation: Δ5′, deletion of 5′exon; Δ3′, deletion of 3′-exons; non, nonoverlapping region; over, 5′UTR overlapping region; flip, overlapping region flipped; ΔM1, partial deletion of MIR; ΔM2 and AM, full deletion of MIR. Units for numbers along the left of gels in b, d, e and f indicate kDa.

FIG. 3—MAPT-AS1 selectively represses tau protein synthesis preventing IRES-mediated translation

a, Schematic representation of constructs used; full-length MAPT 5′-UTR (322 nt) was cloned between Renilla luciferase (Rluc) and Firefly luciferase (Fluc) ORFs into the previously characterized pRF bicistronic vector, resulting into the pRTF construct. Deletion of the 93 nt spanning MAPT-AS1-overlapping region resulted into pRTFΔ; the same t-NAT1-overlapping region was cloned separately to give rise to pRTFover construct. A mutated 5′-TOP motif (CCTCCCCT to AATAAAAT) at positions −243 to −232, relative to the +1 AUG starting codon, resulted into pRTFmTOP construct. A plasmid containing the hepatitis C virus IRES (pRhcvF) was used as a positive control.

b, SH-SY5Y cells stably expressing either an empty vector, t-NAT1 or t-NAT21, were transfected with constructs depicted in (a) and cap-independent translation (Fluc to Rluc ratio) was measured for each reporter. Cells expressing empty vector pcDNA3.1 transfected with pRTF showed a 15-fold increase of the Fluc/Rluc ratio over the negative control pRF vector, and a ˜3.7-fold increase over pRhcvF, providing a basal level of tau IRES activity. In cells expressing either full-length t-NAT1 or t-NAT21, tau IRES activity showed to be significantly reduced (** p<0.01, * p<0.05, one way ANOVA, Dunnett's test n=3). Cells transfected with pRTFΔ or pRTFmTOP showed a reduction in tau IRES activity, but no further decrease with t-NAT expression. In cells transfected with pRTFover, tau IRES activity was similar to the pRF control vector, indicating that the first 229 nt of the 5′-UTR are necessary for tau IRES function.

c, Secondary structure of the MAPT 5′-UTR (from −242 to −1 relative to the AUG) as reported by Veo and Krushel²². Domains I and II of tau IRES are indicated and a blue line indicates t-NAT1 overlapping sequence (5′-exon position 88-163), as previously shown in FIG. 2e. 5′-TOP motif is reported (green).

d, pRTF or pRF construct with either pcDNA3.1 empty vector, t-NAT1 full-length (FL) or a mutant deleted of the inverted MIR repeat (t-NAT1-ΔM) were co-transfected into SH-SY5Y cells, and relative luciferase levels were measured after 48 hours. A significant reduction of tau IRES activity (Fluc/Rluc ratio) was detected in cells expressing t-NAT1-FL, but not t-NAT1-ΔM, which resulted in a significant increase in tau IRES-mediated cap-independent translation.

e, Similarly t-NAT21-FL showed to repress tau IRES activity, whereas t-NAT21-AM, devoid of the inverted MIR repeat, showed to have no such effect. Data in d and e represent mean±s.d., n≥3 (** p<0.01, *p<0.05, one-way ANOVA and Dunnett's test)

f, 13 polysomal fractions, separated on sucrose gradient, were obtained from two independent clones for each cell stably expressing the indicated constructs, and were repeated in two independent experiments. Total RNA was extracted and equal volumes were converted into cDNA, and subjected to qRT-PCR and the percentage of MAPT mRNA in each fraction was calculated (see methods). Bar plot represents relative abundance of MAPT mRNA in pools of fractions corresponding to 40-60S, 80S monosomes, light polysomes, medium weight polysomes and heavy polysomes respectively. Both full-length t-NAT1 and t-NAT21 expressing cells exhibited a significant decrease in the percentage of MAPT mRNA associated to actively translating heavy polysomes. Deletion of the inverted MIR repeat is sufficient to shift tau mRNA into active heavy polysomes, resulting in a net increase in MAPT translation (n=4 for each construct; ** p<0.01, *p<0.05; one-way ANOVA, Dunnett's test). From left to right, the bars represent “Empty”, “t-NAT1-FL”, “t-NAT1-ΔM”, “t-NAT2-FL” and “t-NAT2-ΔM”.

g, Relative abundance of MAPT-AS1 lncRNA, MAPT and β-actin mRNAs in each polysomal fraction. Absorbance profiles (OD at 254 nm) are represented in the background of each plot.

FIG. 4—MIR-lncRNAs form S-AS pairs within a network of interacting protein-coding genes, enriched for neurodegenerative-associated loci

a, MIR repeats of all subfamilies (MIR, MIR3, MIRb, MIRc) constitute a larger fraction of the lncRNAs length than different regions of protein-coding mRNAs (5′-UTR, 3′-UTR, CDS).

b, 1496 lncRNAs annotated in GENCODE v19 contain at least one embedded MIR repeat and form S-AS pairs with 1045 unique protein-coding (PC) genes. Of these S-AS pairs, 40.69% overlap 5′-UTR, 32.50% overlap CDS and 26.81% overlap 3′-UTR.

c, Enriched Gene Ontology (GO)-terms for cellular components and associated diseases as calculated by Enrichr²⁷, are shown for each group of S-AS pairs sorted by the type of exonic overlap (3′-UTR-overlapping, red; 5′-UTR-overlapping, green; CDS-overlapping, blue). PC genes overlapping in 5′-UTR with MIR-lncRNAs are significantly enriched for loci associated to dementia, Parkinson's disease and Amyothrophic lateral sclerosis (** p<0.01, * p<0.05, Benjamini-Hochberg FDR).

d, schematic representation of the human PLCG1 gene overlapping along its first 5′-exon with a MIR-lncRNA (PLCG1-AS) on the opposite strand.

e, Western blot of SH-SY5Y cells stably expressing either an empty vector (Empty), a full-length PLCG1-AS (FL) or its mutant deleted of the MIR repeat (ΔM). PLCG1 protein level is reduced in cells expressing FL-but not ΔM-PLCG1-AS.

f, MIR-lncRNA antisense target genes form an extensive network of interacting proteins (PPI interactions were computed by NetworkAnalyst as a zero-degree interaction network starting from the InnateDB PPI dataset, with 392 seed proteins). Many proteins in this network are encoded by genes associated with neurodegenerative diseases (p=1.63×10⁻⁸, Benjamini-Hochberg FDR, WebGestalt).

g, PC genes overlapping with MIR-lncRNAs along their 5′-UTR are more expressed in human brain as detected by RNA-seq FPKM) when compared to PC genes overlapping in 3′-UTR or CDS (** p<0.01, *** p<0.0001, one way ANOVA across all brain regions).

FIG. 5—Linkage disequilibrium analysis of MAPT-AS1 region

a, SNPs within MAPT-AS1 genomic region (+/−5 kb) that are linked (R²≥0.5) to tagging SNPs from the NHGRI GWAS catalog are reported. The specific trait associated to each tagging SNP together with the P-value from the GWAS study are reported as from cited PubMed publications (references). All P-values 5×10⁻⁸ were considered to be significant. Linkage correlations (R²) were calculated using LDlink1.1 (PMID 26139635) for different populations. ASW: Americans of African Ancestry in SW USA; CEU: Utah Residents (CEPH) with Northern and Western European Ancestry; CHB: Han Chinese in Beijing, China; CHD: Chinese in Metropolitan Denver, Colo.; GIH: Gujarati Indians in Houston, Tex.; JPT: Japanese in Tokyo, Japan; LWK: Luhya in Webuye, Kenya; MXL: Mexican ancestry in Los Angeles, Calif.; MKK: Maasai in Kinyawa, Kenya; TSI: Toscani in Italia; YRI: Yoruba in Ibadan, Nigeria b, For each linked SNP listed in (a), the minor allele frequency (MAF) from the 1000 Genomes Project is reported, together with the exonic/intronic location. c, Pairwise linkage disequilibrium heatmap created using the LDmatrix webserver. Red squares of increasing hue indicate increasing linkage between SNPs. A physical map of the genomic region is reported together with annotated RefSeq transcripts for each gene. d, Enlarged view of the MAPT-AS1 3′-exon (in grey) containing the inverted MIRc element (in green), with two exonic linked SNPs downstream (r517690326, r517763596).

FIG. 6—Evolutionary conservation of t-NAT1 isoform and MAPT-AS1 promoter region across Primates

a, Scheme of the human t-NAT1 transcript isoform composed of two exons (grey), with the MAPT overlapping region (blue) and the inverted MIR element in 3′-end (red). b, Multiple sequence alignment of the human t-NAT1 transcript to the genomic sequence of 10 nonhuman Primates (Baboon, Bonobo, Chimp, Gibbon, Gorilla, Marmoset, Mouse Lemur, Orangutan, Rhesus, Squirrel Monkey). Sequences were aligned using MUSCLE 3.8, and graphically displayed using Jalview 2. Pyrimidines are in cyan and purines in magenta; the splice junction is highlighted in yellow. A consensus sequence is reported at the base of the multi-alignment with a bar plot representing percentage of sequence identity c, Phylogenetic tree associated to t-NAT1 multi-alignment represented in (b), obtained with the neighbor joining method using Jalview 2. Numbers reported on each connecting line in the tree represent Jaccard distances based on pairwise sequence similarity. d, t-NAT1 has a low protein-coding potential as shown by the negative PhyloCSF score. The plot represents the distribution of scores for each codon in each frame within t-NAT1 isoform, across 29 mammals. e, Multi-alignment showing sequence similarity between human t-NAT1 3′-end (388-449) and consensus MIR elements of different subfamilies (MIR3, MIR, MIRb, MIRc) shown as inverted-complement sequences, thus denoted “(-)”, as annotated by RepeatMasker. The homology region of 62 nt map to the CORE-SINE, a 65 nt evolutionary conserved domain at the center of each MIR repeat element, as schematically represented here and originally described by Labuda et al. f, Evolutionary conservation of MAPT-AS1 promoter region across 6 evolutionary distant species (Homo sapiens, Rhesus macaque, Mus musculus, Rattus norvegicus, Canis familiaris, Bos Taurus) was computed using the ECR browser. Exonic regions are in yellow, intronic regions are in orange and repeat elements are in green. Peaks represent identity percentage to the human sequence. On the bottom, CAGE tag clusters from FANTOM4 and FANTOM5 datasets retrieved from the ZENBU genome browser, are mapped to the MAPT-AS1 promoter region, either on the sense (blue) or antisense strand (red). Values on the y-axis represent CAGE counts normalized per million tags (tpm).

FIG. 7—Evolutionary conservation of t-NAT21 isoform across Primates

a, Scheme of the human t-NAT21 transcript isoform composed of four exons (grey) with the inverted MIR element in 3′-end (red). b, Multiple sequence alignment of the human t-NAT21 transcript to the genomic sequence of 9 nonhuman Primates (Baboon, Bonobo, Chimp, Gibbon, Gorilla, Marmoset, Orangutan, Rhesus, Squirrel Monkey). Sequences were aligned using MUSCLE 3.8, and graphically displayed using Jalview 2. Pyrimidines are in cyan and purines in magenta; splice junctions are highlighted in yellow. A consensus sequence is reported at the base of the multi-alignment with a barplot representing percentage of sequence identity c, Phylogenetic tree associated to t-NAT21 multi-alignment represented in (b), obtained with the neighbor joining method using Jalview 2. Numbers reported on each connecting line in the tree represent Jaccard distances based on pairwise sequence similarity. d, t-NAT21 has a low protein-coding potential as shown by the negative PhyloCSF score. The plot represents the distribution of scores for each codon in each frame within t-NAT21 isoform, across 29 mammals. e, Multi-alignment showing sequence conservation between human t-NAT21 3′-end (510-554) and consensus MIR elements of different subfamilies (MIR3, MIR, MIRb, MIRc), shown as inverted-complement sequences, thus denoted “(-)” as retrieved through RepeatMasker. The homology region of 45 nt (red dashed line) is shared with the CORE-SINE, a 65 nt evolutionary conserved domain at the center of each MIR repeat element, as schematically represented here and originally described by Labuda et al.

FIG. 8—RNA-seq read distribution across MAPT and MAPT-AS1 genes in different brain regions

a, RNA-seq read counts for the MAPT mRNA and MAPT-AS1 lncRNA transcripts (t-NAT2s, t-NAT1, t-NAT21) across 12 different regions of four independent human brains. Values represent mean counts+/−s.d. Brain regions are as follows: CBRL, Cerebellum; FCTX, frontal cortex; HIPP, hippocampus; HYPO, hypothalamus; MEDU, medulla; OCTX, occipital cortex; PUTM, putamen; SNIG, substantia nigra; SPCO, spinal cord; TCTX temporal cortex; THAL, thalamus; WHMT white matter.

FIG. 9—Characterization of human induced pluripotent stem cells-derived cortical neurons

a, Control iPSCs were differentiated into cortical neurons using a protocol of dual SMAD inhibition followed by a period of in vitro corticogenesis that generates both deep- and upper-layer cortical excitatory neurons. Neural precursor rosettes at day 20 were positive for primary cortical progenitor markers PAX6 and OTX2, the proliferation marker ki67 and neuronal βIII-tubulin (TUJ1). At this stage, early born neurons started appearing at the periphery of rosettes expressing deep-layer marker TBR1. By day 100, mature neurons had adopted a neuronal morphology, as highlighted by neuronal βIII-tubulin staining, and later-born neurons positive for upper-layer markers SATB2 and BRN2 had developed. Scale bars represent 20 μm. b, Quantitative expression of MAPT and MAPT-AS1 (t-NAT1, t-NAT2s, t-NAT21) in 3 independent inductions of human iPSC differentiated into cortical neurons (from 0 to 100 days in culture) measured by qRT-PCR (ΔΔCt/ΔΔCtmax).

FIG. 10—Expression and nucleo-cytoplasmic localization of endogenous MAPT mRNA are not altered in cells stably expressing MAPT-AS1

a, Normalized MAPT and MAPT-AS1 RNA levels as detected by qRT-PCR from SH-SY5Y cells stably expressing different deletion mutants of MAPT-AS1: t-NAT1 flipped overlapping region (Flip), t-NAT1 non-overlapping region (Non), t-NAT1 overlapping region (Over), tNAT1 deleted of the 5′-exon (t-NAT1Δ5′), tNAT1 deleted of the 3′-exon (t-NAT1Δ3′), tNAT21 deleted of the 5′-exon (t-NAT2Δ5′), tNAT21 deleted of the 3′-exon (t-NAT2Δ3′). Values are normalized to cells stably expressing an empty vector (Empty). Data represent 3 independent biological replicates, with two technical replicas (n=6, mean±s.d.). b, Both full-length (FL) and mutants deleted of the inverted MIR element (ΔM) of MAPT-AS1 isoforms (t-NAT1 and t-NAT2) localise to both cytosol and nucleus without altering the nucleo-cytoplasmic distribution of MAPT mRNA as detected by qRT-PCR. (n≥3, mean±s.d.). c, Silencing MAPT-AS1 does not alter significantly MAPT mRNA level in SH-SY5Y cells transiently transfected with isoform-specific siRNAs (si-NAT1, si-NAT2) or an siRNA common to all isoforms (si-Ex4) targeting a shared exon in 3′-end. Data represent relative gene expression detected by qRT-PCR and normalized to the control siRNA-treated cells (n=3, mean±s.d.).

FIG. 11—Expression level of MAPT protein in cells stably expressing MAPT-AS1 with a flipped MIR element

a-c, Western blots probed with anti-MAPT and anti-β-actin antibodies. Total protein lysates (20 μg) from independent clones of SH-SY5Y cells stably expressing different isoforms of MAPT-AS1, either full-length (t-NAT1-FL, t-NAT2-FL), deleted of the inverted MIR element (t-NAT1-ΔM, t-NAT2-ΔM) or containing a flipped MIR repeat (t-NAT1-Mflip). Samples in a, b, c represent independent biological replicates. d, Total tau protein normalized to β-actin levels, as quantified using ImageJ, is reported for each type of construct being expressed (* p<0.05, ** p<0.01, *** p<0.001; one-way ANOVA, Dunnett's test; n=6). Similarly to the deletion of the entire MIR repeat (t-NAT1-ΔM), flipping direction of the MIR repeat within t-NAT1 lncRNA (t-NAT1-Mflip, indicated by the red lines) results in an increased tau protein level.

FIG. 12—MAPT-AS1 have no significant effect on MAPT 3′-UTR

a, Schematic representation of the luciferase constructs (pMIR-reporter) used to study MAPT-AS1 effects on MAPT 3′-UTR following transfection in SH-SY5Y cells. Either the full-length (FL) or 3 partially overlapping fragments (Fr1, Fr2, Fr3) of MAPT 3′-UTR were cloned downstream of the Firefly luciferase ORF. b, Firefly luciferase (Fluc) normalized to Renilla luciferase (Rluc) was quantified in SH-SY5Y cells co-transfected with either an empty pcDNA3.1 vector or different versions of t-NAT1 antisense-lncRNA, (n=6, 2 experiments). c, Fluc to Rluc ratio was quantified in SH-SY5Y cells co-transfected with either an empty pcDNA3.1 vector or different versions of t-NAT21 antisense-lncRNA. (n=6, 2 experiments). In all cases differences were not statistically significant.

FIG. 13—Brain RNA-seq co-expression analysis

a, Co-expression heatmaps representing distribution of RNA-seq read counts for the top 100 most abundant MIR-lncRNA target protein-coding genes (on the left side) and the top 100 most abundant MIR-lncRNA genes (on the right side), both hierarchically clustered based on their expression level in 12 different regions of 4 independent post-mortem brains from healthy human donors. Genes are clustered on the y-axis. Brain regions, reported on the x-axis, are as follows: CBRL, Cerebellum; FCTX, frontal cortex; HIPP, hippocampus; HYPO, hypothalamus; MEDU, medulla; OCTX, occipital cortex; PUTM, putamen; SNIG, substantia nigra; SPCO, spinal cord; TCTX temporal cortex; THAL, thalamus; WHMT white matter. For each brain region, 4 independent brain samples are represented in each column. A color key with histogram relative to each heatmap, have z-values associated to each color on the x-axis and RNA-seq counts on the y-axis. The histogram represents distribution of the RNA-seq counts for each z-value. b, Similar co-expression heatmaps, as in (a), representing 1045 MIR-lncRNA target protein-coding genes (on the left side) and 1197 antisense MIR-lncRNA genes (on the right side). c, Pie chart showing the percentage of MIR-lncRNA S-AS pairs annotated in GENCODE v19 and overlapping in 5′-UTR, sorted by their Pearson's correlation coefficient. The majority of S-AS pairs show a positive correlation. d, Histogram representing frequency of occurrence for 1197 MIR-lncRNA S-AS pairs in bins of Pearson's correlation (from −1 to +1 in bins of 0.05). All MIR-lncRNA S-AS are visualized together, irrespective of their pattern of overlapping. MAPT-AS1-MAPT correlation coefficient is indicated.

FIG. 14—Genes paired with antisense MIR-lncRNAs have significantly more structured 5′- and 3′-UTRs 3′-UTR (a) or 5′-UTR (b) minimum free energy (MFE), normalized by its length was computed using RNAfold 2.1.9 for each protein-coding gene in the human genome (hg19), and sorted based on their respective type of lncRNA overlap. Boxplot presents median, upper and lower quartile boundaries for each group of protein-coding (PC) genes. PC genes pairing with MIR-lncRNAs have both 3′-UTR and 5′-UTR significantly more structured that PC genes with no lncRNA overlap (***, p<0.0001 one-way ANOVA, Dunnett's test). PC genes groups are as follows: PC genes overlapping antisense MIR-lncRNA, PC-MIRlncRNA; PC genes overlapping any lncRNA without embedded MIR repeat, PC-lncRNA-NOMIR; all PC genes with any overlapping lncRNA, PC-lncRNA; MIR-lncRNAs, MIRlncRNA; PC genes without lncRNA overlap, PC-NO-lncRNA.

FIG. 15—Majority of genes targeted by antisense MIR-lncRNAs interact in a PPI network and are enriched for neurodegenerative disease-associated proteins

a, Protein-protein interaction (PPI)-network obtained mapping literature-curated interactions data from the InnateDB database, using 392 seed proteins participating in S-AS pairs with MIR-lncRNAs. Genes encoding for proteins associated to neurodegenerative diseases, represented as red-filled circles, are significantly enriched into the network (p=1.63×10⁻⁸, Benjamini-Hochberg FDR using WebGestalt). Only primary interactions are represented in a zero-degree interaction network generated using the NetworkAnalyst tool. Self-interactions are not considered. b-e, Schematic structures of representative genes pairing with antisense MIR-lncRNAs and involved in different neurodegenerative diseases. GENCODE annotated isoforms of the human SNCA (b), APP (c), MBNL1 (d) and SLC1Δ2 (e) genes together with their respective overlapping antisense MIR-lncRNA. MIR elements (red) positions within each lncRNA are indicated.

FIG. 16—Majority of genes targeted by antisense MIR-lncRNAs interact in a PPI network and are enriched for immune system-associated genes

a, Protein-protein interaction (PPI)-network obtained mapping literature-curated interactions data from the InnateDB database, using 392 seed proteins participating in S-AS pairs with MIR-lncRNAs. Genes encoding for proteins associated to either immune system (green) or innate immune system (purple), are significantly enriched into the network (respectively p=0.0041, p=0.0328, Benjamini-Hochberg FDR using NetworkAnalyst). Only primary interactions are represented in a zero-degree interaction network generated using the NetworkAnalyst tool. Self-interactions are not considered. b, Gene expression heatmap for 487 protein-coding genes overlapping along 5′-UTR with antisense MIR-lncRNAs in 126 normal human tissues, from 557 publicly available microarray datasets, retrieved from the Enrichment Profiler Database (http://xavierlab2.mgh.harvard.edu/EnrichmentProfiler/index.html) Genes are clustered on the y-axis and tissues are clustered on the x-axis. The scale bar on the bottom indicates colors associated to each Z-score in the expression heatmap.

FIG. 17—Distribution of 7-mer MIR-complementary motifs along the human 18S rRNA secondary structure

The human 18S ribosomal RNA secondary structure as retrieved from (http://apollo.chemistry.gatech.edu/RibosomeGallery/) is divided into an “active region” (red) and an “inactive region” (grey). As described in Weingarten-Gabbay S. et al. 2016⁽²⁵⁾, the active region is enriched for motifs able to mediate 40S ribosome recruitment through direct RNA-RNA interactions with 5′-UTRs of about 10% of human genes. Here the 18S rRNA secondary structure is superimposed to 7-mers of complementary motifs (black dots) contained within each MIR element embedded in MIR-lncRNAs overlapping in 5′-UTR with PC genes. Only 7-mers complementary to the 18S active region are shown. The 7-mer motifs represented here map to both the MIR elements within antisense MIR-lncRNAs and the 5′-UTRs of the respective target genes, as reported in detail in Table 1.

FIG. 18—Model for inverted MIR-mediated repression of internal initiation

a, Contour line representing the human 18S rRNA secondary structure with the active region (red) and the inactive region (black), and two 7-mer motifs complementary to positions 53-59 and 102-108 within MAPT IRES, mapping respectively to stem 21es6d and at the basis of helix 33. b, In absence of MAPT-AS1 lncRNA, MAPT IRES is active and able to actively recruit the ribosome, potentially through a direct RNA-RNA interaction mediated by two 7-mer motifs complementary to a bulge region within domain 1 (red, 53-59 nt) and to a single strand loop connecting domain 1 to domain 2 (blue, 102-108 nt). Furthermore nucleotides 59-65 and 19-25 (black dots) are complementary to each other and their spatial proximity through a kissing-hairpin interaction, has been reported to be crucial for tau IRES activity. This may lead the tau IRES to assume a complex tertiary conformation and bringing rRNA-complementary regions in close vicinity, it might favor interaction of the 40S ribosome with the AUG starting codon. c, In the presence of MAPT-AS1, MAPT IRES is repressed, and this requires the presence of both a 5′-region complementary to the domain 2 (blue line) and the MIR element in 3′-end (purple thick line) of MAPT-AS1. The inverted MIR element embedded within MAPT-AS1 contains at least two conserved 7-mers, one (CACCCAC, blue) complementary to the same rRNA site at the basis of helix 33 (grey lines), and the other (CTGAGGC, red) identical to the 18S rRNA motif in stem 21esd6, which can mediate IRES repression due to a direct competition for pairing with the rRNA. The same strategy may explain a more widespread mode of action of embedded MIR elements within other antisense MIR-lncRNAs onto their target genes. Conversely the presence of a MAPT-AS1 deleted of the MIR element, leaves the 5′-region the only domain of the lncRNA able to pair with domain 2 of tau IRES (b, blue line), potentially stabilizing it in a more open conformation, favoring its interaction with rRNA.

FIG. 19—Expression of tNAT1, tNAT2 and IT1 in brain and in human neuroblastoma cell lines

a, The overlap of tNAT1, tNAT2 and IT1 with the MAPT promoter, particularly with the core promoter, is shown. Genomic region represented shows the MAPT 5′ promoter domain from core promoter at exon 0 (red arrow box, lower line, centre-left) to first coding exon 1 (blue box, labelled “MAPT exon 1”) and conserved downstream repressor domain (green oval, lower line, besides exon 1) containing rs242557. IMP5 gene is upstream to MAPT promoter. Non-coding RNA genes are shown above bold line. Relative distances (in kilobases are indicated, top. b, c, Reduction of tau levels with transient expression of MAPT-associated lncNRAs. Arrows indicate reduced tau protein levels. Deletion variants of t-NAT1 (NT1D5 and NT1D3) do not reduce tau levels.

FIG. 20—Reduction of tau protein levels with stable t-NAT expression

The effects of three independent clones overexpressing tNAT1 (NT1-1, NT1-2 and NT1-3) whereby tau protein levels are almost completely eliminated compared to empty vector clones (V5) and clones expressing variants of tNAT1 with deletions or rearrangements of the 5′ exon of the tNAT1 that overlaps with the MAPT promoter or the distal 3′ region of tNAT1.

a, Reduction of tau protein levels with stable t-NAT2. Arrows indicate reduced tau protein levels in independent clones expressing wild-type t-NAT1 compared to empty vector (V5) and deletion variants.

b, Reduction of tau protein levels with stable t-NAT1 and t-NAT2. Arrows indicate reduced tau protein levels in independent clones expressing wild-type t-NAT1 compared to empty vector (V5) and deletion variants. Variants with deletion of regions overlapping the MAPT promoter lose tau expression suppression activity, showing the importance of this overlap.

FIG. 21—Model of translational repression of MAPT mRNA by t-NAT1: a, schematic representation of the MAPT mRNA with its 5′-UTR secondary structure, as experimentally determined by Veo and Krushel, 2012. In the absence of MAPT-AS1, the MAPT IRES can recruit efficiently the 40S ribosomal subunits (green ovals), potentially by direct pairing with the 18S rRNA at two 7-mer complementary sites (red, 53-59 nt; blue, 102-108 nt; thick lines). Furthermore, nucleotides 59-65 and 19-25 (black dots) are complementary to each other and their spatial proximity through a kissing-hairpin interaction, has been reported to be crucial for tau IRES activity (Veo and Krushel [22]).

b, In the presence of MAPT-AS1 (t-NAT1), MAPT IRES is repressed, and this requires the presence of both a 5′-region complementary to the domain 2 (blue line) and the MIR element in 3′-end (purple thick line) of MAPT-AS1. The inverted MIR element embedded within MAPT-AS1 contains at least two conserved 7-mers, one (CACCCAC, motif 1) complementary to the same rRNA site at the basis of helix 34, and the other (CTGAGGC, motif 2) identical to the 18S rRNA motif in stem 21esd6, which can mediate IRES repression due to a direct competition for pairing with the rRNA.

FIG. 22—Functional features of t-NAT1: The t-NAT1 transcript (449 bp) contains two essential sequences, the 5′ region of antisense overlap with MAPT 5′UTR (AS; blue box, towards the left-hand side of the lower-left panel) and the MIR domain (red boxes at the right-hand side of the lower-left panel). The conserved MIR domain contains three 7-mer motifs (black boxes, Motif 1, Motif 2 and Motif 3, numbered in small black boxes both in the top panel and within the red boxes in the lower left panel) that mediate MAPT mRNA ribosomal interaction and translation. Western blots for tau protein (lower right panels) in cell lines overexpressing MAPT or β-actin (control) sequences confirm that, full-length t-NAT1 represses tau protein levels whereas deletion of Motif 1 and Motif 2 (but not of Motif 3), reduces this repression. Sequence of conserved MIR domain with the three motifs are shown at top of figure. The bottom bar depicts the MiniNAT that contains only the region of overlap (AS) and the MIR. Western blot (left) of stably overexpressed MiniNAT shows significantly reduced tau protein. MAPT knockdown is most pronounced in the bottom two bars and in the first bar below the empty vector control.

### DETAILED DESCRIPTION

The following applications of the present invention are provided by way of example and not limitation.

### MAPT-AS1

The present inventors have characterised MAPT-AS1, as a 5′ antisense long non-coding RNA (lncRNA) gene with head-to-head orientation overlapping with MAPT 5′-UTR. MAPT-AS1 extends for ˜52 kilobases upstream from MAPT (FIG. 1a) and resides within the extended region of linkage disequilibrium (LD) that defines the H1 and H2 haplotypes¹⁴ (Extended Data, FIG. 1a, 1b).

The inventors found an inverted MIR element within MAPT-AS1, which is required for its repressive activity, and they found that deletion or inversion of the MIR element converts MAPT-AS1 into an enhancer of tau translation. Complementarity between MAPT-AS1 and the internal ribosome entry site (IRES), within the 5′-untranslated region (5′-UTR) of MAPT mRNA was also found to lead to correlate with translationally repressive activity.

### MAPT-AS1 Transcripts (NATs)

The inventors identified three alternative splicing isoforms, referred to as tau-Natural Antisense Transcripts t-NAT1, t-NAT2s, t-NAT21. These tau-Natural Antisense Transcripts are associated with two alternative transcription start sites (TSS), with t-NAT2s and t-NAT21 each being associated with a TSS located in intron 1 of MAPT, with t-NAT1 being associated with a TSS that overlaps the 5′-untranslated region (5′-UTR), of MAPT (FIG. 1a).

```
MAPT-AS1 transcript sequences
t-NAT1 [449 bases]
[SEQ ID NO: 11]
                10 20 30 40 50

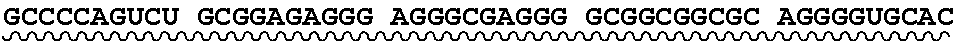

                60 70 80 90 100

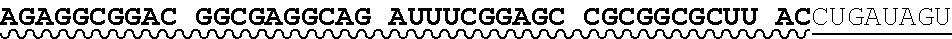

              110 120 130 140 150
CGACAGAGGC GAGGACGGGA GAGGACAGCG GAGGAGGAGA AGGUGGCUGU
              160 170 180 190 200
GGUGGCGGCG GCAGAAGGAG UCAGAACAAG GACGGGCCUG UGAUGUGUCU
              210 220 230 240 250
GUCUAUGACG AGGAGGACAA UGUCCUAAGG AAUGGAGAGG AAGAAAGUGA
              260 270 280 290 300
AGGGAACCAG GUCCCUGGAU UCCAUGUGGA ACAGUAGCCC AGGAUGUGCA
              310 320 330 340 350
CUAUGGACUG UUACAAGAGA GAGACAUGAU CUUCCUUAAG GCAUUGAUUU
              360 370 380 390 400

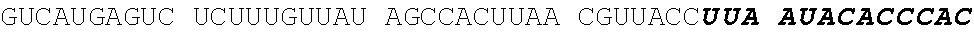

              410 420 430 440

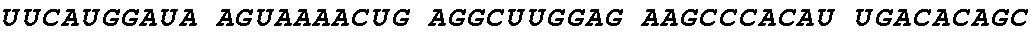

```

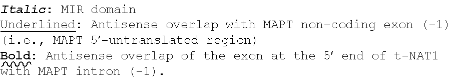

tau-NAT1 exon 1: 1-167
tau-NAT1 exon 4: 168-449

```
t-NAT2L [544 bases]
[SEQ ID NO: 12]
                10 20 30 40 50

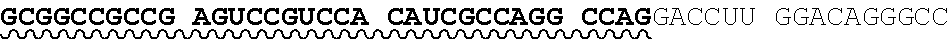

                60 70 80 90 100
CAGCACAGGG GCCCUGGAAU GUGGACUGUC UCAGUGGAUU CUUGUUUAUA
              110 120 130 140 150
GGAAUUAGAG GAAGGUGGAA GAAGCUCAUU CCAGAGUGCA AUCAAUAACA
              160 170 180 190 200
GUGGAAAGAA GUCCCUGGAG CCCAGUGCAG UGGCUUGAAC UGAGAAUGCA
              210 220 230 240 250
CUCACUGGCU GUUGCAGCCA AGACUCCAGU UCUCGCCUCU CUUUCCCUGA
              260 270 280 290 300
CUCCCCUGUG CCCACCUUUC CCCUGCAGGA GUCAGAACAA GGACGGGCCU
              310 320 330 340 350
GUGAUGUGUC UGUCUAUGAC GAGGAGGACA AUGUCCUAAG GAAUGGAGAG
              360 370 380 390 400
GAAGAAAGUG AAGGGAACCA GGUCCCUGGA UUCCAUGUGG AACAGUAGCC
              410 420 430 440 450
CAGGAUGUGC ACUAUGGACU GUUACAAGAG AGAGACAUGA UCUUCCUUAA
              460 470 480 490 500

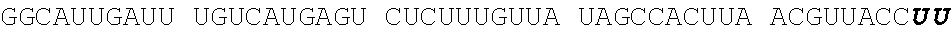

              510 520 530 540

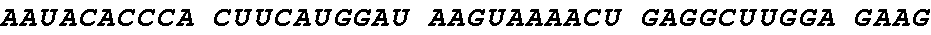

```

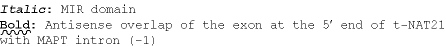

tau-NAT2L exon 1: 1-34
tau-NAT2L exon 2: 35-134
tau-NAT2L exon 3: 135-278
tau-NAT2L exon 4: 279-544

```
t-Nat2S [924 bases]
[SEQ ID NO: 13]
                10 20 30 40 50

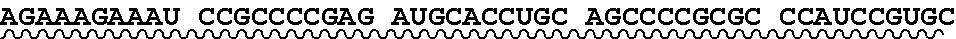

                60 70 80 90 100

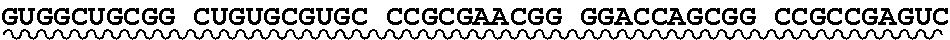

              110 120 130 140 150

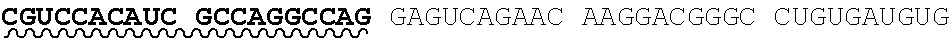

              160 170 180 190 200
UCUGUCUAUG ACGAGGAGGA CAAUGUCCUA AGGAAUGGAG AGGAAGAAAG
              210 220 230 240 250
UGAAGGGAAC CAGGUCCCUG GAUUCCAUGU GGAACAGUAG CCCAGGAUGU
              260 270 280 290 300
GCACUAUGGA CUGUUACAAG AGAGAGACAU GAUCUUCCUU AAGGCAUUGA
              310 320 330 340 350

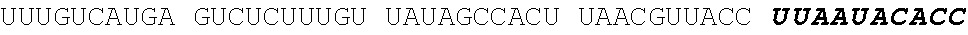

              360 370 380 390 400

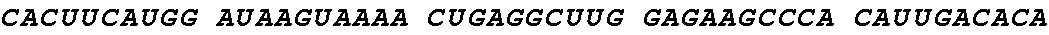

              410 420 430 440 450

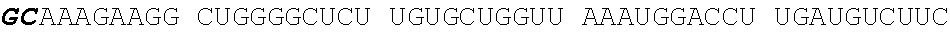

              460 470 480 490 500
CAAAUGUUUU CAUGCUGGAU UCUGAGCCAC UCUCCUGGCU GCAACUCUGA
              510 520 530 540 550
GGCCGGGCAG ACUUUCCGCU GGAAAGAGAA CUCAAAUACU GGUGAGAGGU
              560 570 580 590 600
GGAUUUGAAG CCAGGAGCCC CAGGUUUUGG CUCUGCAGGU UUGCACCCAG
              610 620 630 640 650
CUCUGUGGUG UAUGCCCUCA CAGGUCACCC AGGACCUCCU GUCCAGGCUU
              660 670 680 690 700
CUUCAGGCCA CCCAUGAUGG AGUAGAUUUG AGGAAUGAGG UUUUCAAAGC
              710 720 730 740 750
AGUUAGUGUG CGGGAGAGUA ACCCUGGGGC CUCCGGAACC AGAAGGGAGG
              760 770 780 790 800
GAUUUGGGGG CUGGAGCUUG GCAGUCCAGG UUCCAUUGUC CUCAUGUUCU
              810 820 830 840 850
CCCCACUGGC CUGGCCUGUC ACUCCAGUCU CUGCAUAGGU UGUCACAUGG
              860 870 880 890 900
CAUUCUCCCU GUGUGUCUCU GUGUCUCUUC UCUUCUUUAU AAAGACAUAG
              910 920
UUAUAAUGGA UUAGGGCCCA CCCU
```

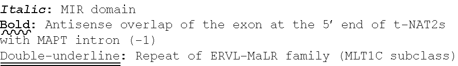

tau-NAT2s exon 1: 1-120
tau-NAT2s exon 4: 121-924

A schematic showing the position of the exons of t-NAT1, t-NAT2s and t-NAT21 in relation to MAPT is shown in FIG. 19a.

The portion of the DNA encoding the 5′ exon of t-NAT1 that overlaps with MAPT intron −1:

[SEQ ID NO: 15]

GCCCCAGTCT GCGGAGAGGG AGGGCGAGGG GCGGCGGCGC
AGGGGTGCAC AGAGGCGGAC GGCGAGGCAG ATTTCGGAGC
CGCGGCGCTT AC

The portion of the DNA encoding the 5′ exon of t-NAT1 that overlaps with MAPT non-coding exon (−1) (MAPT 5′-UTR):

[SEQ ID NO: 14]

CTGATAGTCG ACAGAGGCGA GGACGGGAGA GGACAGCGGA
GGAGGAGAAG GTGGCTGTGG TGGCGGCGGC AGAAG

The portion of the DNA encoding 5′ exon of t-NAT21 that overlaps with MAPT intron (−1)

[SEQ ID NO: 16]

GCGGCCGCCG AGTCCGTCCA CATCGCCAGG CCAG

The portion of the DNA encoding 5′ exon of t-NAT2s that overlaps with MAPT intron (−1)

[SEQ ID NO: 17]

AGAAAGAAAT CCGCCCCGAG ATGCACCTGC AGCCCCGCGC
CCATCCGTGC GTGGCTGCGG CTGTGCGTGC CCGCGAACGG
GGACCAGCGG CCGCCGAGTC CGTCCACATC GCCAGGCCAG

### Other MIR-lncRNAs

The inventors also found that AS-lncRNAs (besides MAPT-AS1) containing the MIR element (MIR-lncRNAs) often have reciprocal expression in central nervous system and immune cells, and often overlap with genes implicated in neurodegenerative disorders and encoding interacting proteins. To demonstrate that the embedded MIR repeats within AS-lncRNAs (besides MAPT-AS1) can repress translation of other proteins (besides tau), the inventors experimentally validated an MIR-lncRNA overlapping with the first 5′-exon of the human gene encoding for phospholipase c gamma 1 (PLCG1) (FIG. 4d). Cells expressing the antisense MIR-lncRNA (PLCG1-AS FL) showed robust reduction of PLCG1 protein (FIG. 4e).

### Genomic Location of MIR-lncRNAs

As described herein, the therapeutic RNAs of the invention comprise one or more sequences that correspond with a MIR-lncRNA. The MIR-lncRNA gene is antisense to a target gene (e.g., a protein-coding target gene). Hence, the MIR-lncRNA gene can be denoted AS-MIR-lncRNA or MIR-AS-lncRNA. Designation as ‘AS-lncRNA comprising a MIR domain’, or similar, can also be used.

The AS-MIR-lncRNA either overlaps with the target gene or the AS-MIR-lncRNA is positioned in the genomic region immediately adjacent to the target gene. Where the AS-MIR-lncRNA overlaps with the target gene, the AS-MIR-lncRNA may comprise an exon at the 5′ end of the AS-MIR-lncRNA gene that overlaps with an untranslated region of the target gene, or an intron of the target gene or a coding sequence of the target gene.

An overlapping sequence in the context of an AS sequence is complementary to whatever sequence that it overlaps with. The term “overlap” refers to any degree of overlap. For readability, the words “at least partial overlap” or similar may also be used, without any change in the meaning of “overlap” being implied where this term is used without a qualifier.

In some embodiments the AS-MIR-lncRNA overlaps with an untranslated region of the target gene. In some embodiments the AS-MIR-lncRNA overlaps with an intron of the target gene. In some embodiments the AS-MIR-lncRNA overlaps with a coding sequence of the target gene. In any of these three embodiments, it may be the 5′ exon of the AS-MIR-lncRNA that overlaps with the recited part of the target gene.

Where the AS-MIR-lncRNA is positioned in the genomic region immediately adjacent to the target gene, the distance between the transcriptional start site (TSS) of the AS-MIR-lncRNA and the TSS of the target gene may be less than 1000 kb, less than 800 kb, less than 500 kb, less than 300 kb, less than 200 kb, less than 100 kb, less than 80 kb, less than 50 kb, less than 30 kb, less than 20 kb, less than 10 kb, less than 8 kb, less than 5 kb, less than 3 kb, less than 2 kb or less than 1 kb.

The location of MAPT-AS1 relative to MAPT is shown in FIG. 1A. MAPT-AS1 overlaps with MAPT. The t-NAT1 transcript has a 5′ exon that overlaps with the 5′ UTR of MAPT and with the first intron of MAPT. The t-NAT21 and t-NAT2s transcript each have a 5′ exon that overlaps with the first intron of MAPT.

### Use of the Therapeutic RNA of the Invention

The therapeutic RNA of the invention can be used in methods of treatment of the human or animal body by therapy. Methods of treatment by administration of a vector of the invention, or administration of the therapeutic RNA of the invention are hereby disclosed. The method of treatment may include administration of the therapeutic RNA of the invention to a subject. The subject may be a mammalian subject. The subject may be a human. The subject may be suffering from a disease, e.g. a neurodegenerative disease. The subject may be suffering from a tauopathy such as Alzheimer's disease (A.D.).

The therapeutic RNA of the invention may be administered to a subject, e.g. by intravenous administration, intracranial administration, by injection into the CSF, by transdermal administration or by oral administration. The skilled person will appreciate that the vectors that deliver or express the therapeutic RNA of the invention can similarly be administered by these or other routes (e.g. intramuscular administration or by administering a sub-dermal dose).

The therapeutic RNA of the invention may be administered as a pharmaceutical preparation (e.g. as a tablet). The pharmaceutical preparation will typically include one or more pharmaceutically acceptable excipients.

The therapeutic RNA of the invention may be administered in combination with one or more other therapies. For example, where the therapeutic RNA of the invention is used in the treatment of tauopathies, the therapeutic RNA may be administered together with one or more other therapeutic agents that reduce tau aggregation.

Where the therapeutic RNA of the invention is used in the treatment of Alzheimer's disease, it may be used in combination with cognitive, behavioral or psychosocial therapies and/or in conjunction with one or more agents used to alleviate the symptoms of Alzheimer's disease.

The therapeutic RNA of the invention may be used prophylactically to modulate the gene expression levels of subjects at risk of developing a condition. For example, the therapeutic RNA of the invention may be administered prophylactically to patients at risk of contracting one or more neurodegenerative diseases for example a tauopathy such as Alzheimer's disease.

The therapeutic RNA of the invention finds uses in any application in which the expression of a target gene is to be modulated. For example, the therapeutic RNA of the invention may be used clinically, or in industrial or academic research.

Introduction of MAPT-AS1 into Genetically Engineered Organisms

Recent advances in genetic engineering techniques, e.g. those made available by CRISPR/Cas9 technology, allow the MAPT-AS1 gene to be inserted into model organisms that do not have an endogenous copy. The skilled person understands that genetically engineered cells comprising additional copies of the MAPT-AS1 gene can be readily produced. The skilled person also understands that non-human animal models can be readily produced, in which the genetically engineered non-human animal has extra copies of the MAPT-AS1 gene. Such genetically engineered cells and non-human animals form a part of this invention.

Similarly, the skilled person will understand that the methods of the invention can be used to specifically express particular transcripts of the MAPT-AS1 gene in genetically engineered cells and genetically engineered non-human animals. For instance, CRISPR/Cas9 technology or integrating viral vectors (e.g. lentiviral vectors) can be used to introduce cDNA that expresses any one of t-NAT1, t-NAT2S or t-NAT2L into the genome of a cell.

The skilled person will appreciate that the genetically engineered cell can be used to produce genetically engineered non-human animals that express any one of t-NAT1, t-NAT2S or t-NAT2L.

Such genetically engineered cells and non-human animals allow the study of tau biology and enable further characterisation of the therapeutic effects of MAPT-AS1 overexpression.

Section Headings

The section headings used throughout this disclosure are for the sole purpose of aiding readability and are not to be construed as limiting in any way.

### Definitions

### Therapeutic RNA

The therapeutic RNA of the invention may be chemically-produced or may be expressed (transcribed) from another nucleic acid. Where the therapeutic RNA of the invention is expressed from another nucleic acid, this may occur in a producer cell or it may occur within the target cell. Where the therapeutic RNA of the invention is produced outside the target cell (i.e. where it is chemically-produced or where it is expressed by a producer cell), the therapeutic RNA of the invention may be chemically-modified following its extraction/purification and prior to its use in the methods of the invention. For instance, the therapeutic RNA of the invention may be labeled and/or chemically-modified to enhance half-life and/or pharmacokinetic properties. In some embodiments, the therapeutic RNA of the invention is chemically-produced using modified nucleotides.

The skilled reader will appreciate that the word ‘therapeutic’ does not limit the use of the therapeutic RNA of the invention. The therapeutic RNAs of the invention find utility in the modulation of gene expression in all types of research, clinical and industrial applications. It is intended that any RNA molecule conforming to the structural and functional definitions of the claims can be considered to be a ‘therapeutic RNA of the invention’ even when not directly used in therapeutic applications.

### Modulation of Gene Expression

The present invention uses therapeutic RNA to modulate gene expression. Gene expression can be repressed or enhanced. The therapeutic RNA of the present invention modulates gene expression at the translational level. The skilled person will therefore understand that modulation of gene expression according to the present invention means that translation is modulated without a substantial corresponding modulation of gene transcription. Hence the present invention can be used to repress gene translation without substantially repressing gene transcription. Alternatively, the present invention can be used to enhance gene translation without substantially enhancing gene transcription.

### Other Nucleic Acid Molecules of the Invention

As described herein, this invention can be practiced by delivering into cells nucleic acid molecules other than RNA (such as DNA, modified nucleic acids or nucleic acid analogues) that lead to the expression of the therapeutic RNA of the invention in the cell. Expression of the therapeutic RNA of the invention by other nucleic acid molecules of the invention may be under the control of a tissue-specific promotor or a promoter that can be activated or switched off by application of external stimuli.

### Overlap

An overlapping sequence in the context of an AS sequence is a sequence that is complementary to whatever sequence that is stated to overlaps with it. The term “overlap” refers to any degree of overlap. Therefore, an AS-lncRNA gene overlaps a protein-coding gene if at least one base of the AS-lncRNA gene is complementary with at least one base of the protein-coding gene in situ in the genome. For readability, the words “at least partial overlap” or similar may also be used, without any change in the meaning of “overlap” being implied where this term is used without a qualifier.

### Vectors

This disclosure provides vectors for delivering to cells, or for expressing in cells, the therapeutic RNA of the invention. The term ‘vector’ is to be interpreted broadly, to include viral vectors and nonviral vectors. Nonviral vectors include plasmid vectors. The skilled person will appreciate that any means of delivering a therapeutic RNA of the invention to a target cell can be used as a vector.

The skilled person will appreciate that (i) ‘RNA vectors’ can be used to transfect or transduce the RNA of the invention directly into a target cell, or (ii) ‘DNA vectors’ can be used to transfect or transduce another nucleic acid (e.g. a DNA molecule, a modified DNA or DNA analogue), which expresses the RNA of the invention in the target cell. The skilled person will appreciate that transduction usually refers to the use of a viral vector while transfection usually refers to the use of a nonviral vector.

A wide range of vectors are known in the art, which can be used to deliver the RNA of the invention to a target cell as described above, either (i) ‘directly’ or (ii) by delivering a nucleic acid that encodes the RNA of the invention and expresses it in the target cell.

Methods for Gene Transfer to the Central Nervous System is the subject of reference³³, which is herein incorporated by reference in its entirety.

The following vector types are discussed as non-limiting examples of vectors that may be used to apply the present invention. The skilled person will appreciate that other vectors capable of delivering the therapeutic RNA of the invention to a target cell may also be used.

Adeno-associated virus (AAV) vectors deliver DNA to a transduced cell. Hence, AAV vectors can be used to deliver into cells a DNA molecule that expresses the therapeutic RNA of the invention. AAV has been the predominant choice for central nervous system-focused clinical trials³⁴. The AAV vector may be integrating or non-integrating. The AAV vector may be pseudotyped to increase transduction efficiency and/or to increase target cell specificity. The aav vector may be targeted to particular cell types, such as neurones. The AAV vector may be based on AAV9.

Adenoviral vectors deliver DNA to a transduced cell. Hence, adenoviral vectors can be used to deliver into cells a DNA molecule that expresses the therapeutic RNA of the invention. Typically, adenoviral vectors are non-integrating. The adenoviral vector may be pseudotyped to increase transduction efficiency and/or to increase target cell specificity. The adenoviral vector may be targeted to particular cell types, such as neurones.

Retroviral vectors are based on RNA viruses and can be used to deliver into cells the therapeutic RNA of the invention directly, or a nucleic acid molecule that expresses the therapeutic RNA of the invention. Most commonly, retroviral vectors are used to deliver an RNA molecule that expresses the therapeutic RNA of the invention in the target cell, following a process of reverse transcription.

Retroviral vectors include lentiviral vectors. Lentiviral vectors, e.g. HIV-based vectors, may be integrating or non-integrating. The retroviral vector may be pseudotyped to increase transduction efficiency and/or to increase target cell specificity. The retroviral vector may be targeted to particular cell types, such as neurones.

Herpes simplex virus (HSV) delivers DNA to infected cells. Hence, HSV-based vectors can be used to deliver into cells a DNA molecule that expresses the therapeutic RNA of the invention. The HSV-based vector may be integrating or non-integrating. HSV has a natural tropism for neuronal cells. HSV-based vectors can be pseudotyped to increase transduction efficiency and/or to increase target cell specificity. The HSV-based vector may be targeted to particular cell types, such as neurones.

Naked DNA/plasmid vectors can be used to deliver DNA encoding the therapeutic RNA of the invention into a target cell. The naked DNA/plasmid vector comprises a DNA sequence encoding the therapeutic RNA of the invention, operably linked to a promoter that can be functional in the target cell. The therapeutic RNA of the invention is thereby expressed by the naked DNA/plasmid vector in the target cell. The skilled person will appreciate that plasmid vectors are circular DNA molecules and may be themselves considered as naked DNA vectors if they are not associated with another chemical entity that assists cell entry. However, plasmid vectors can be linearised prior to transfection (this can enhance genomic integration of the plasmid). Other naked DNA vectors besides plasmids (linear DNA molecules, cosmids, etc) are also well-known.

Delivery of naked DNA/plasmid vectors into cells can be achieved by well-known means such as electroporation, sonoporation or by delivering gold nanoparticles coated the a with a plasmid vector into the cell e.g. using a ‘gene gun’. Plasmid vectors can also be associated/complexed with chemical entities such as antibodies, saccharide moieties and/or lipids to enhance cell entry. The association can be via covalent bonds or via non-covalent interactions. In this way, plasmid vectors can be targeted to particular cell types, such as neurones.

Nanoparticle vectors can be used to deliver into cells the therapeutic RNA of the invention directly, or a nucleic acid molecule that expresses the therapeutic RNA of the invention. Nanoparticle vectors include gold nanoparticles, silica nanoparticles, carbon nanoparticles, calcium phosphates, lipid nanoparticles and quantum dots. Lipid nanoparticles designed to deliver the therapeutic RNA directly to specific cells such as neurones may be used. Multi-layered nanoparticles, in which components intended to protect the therapeutic nucleic acid and/or target the nanoparticle to the cell may also be used with this invention. Nanoparticles may be functionalised with further components to enhance cell targeting and/or may deliver further therapeutic agents to the cell in addition to the therapeutic nucleic acid of the invention. Nanoparticles may be targeted to particular cell types, such as neurones.

Dendrimers can be used as vectors to deliver into cells the therapeutic RNA of the invention directly, or a nucleic acid molecule that expresses the therapeutic RNA of the invention. Dendrimers are highly branched macromolecules with an (approximately) spherical shape, which can be functionalised with the therapeutic RNA of the invention and/or another nucleic acid molecule expressing the therapeutic RNA of the invention. Dendrimers are taken into the target cell by endocytosis and may be targeted to particular cell types, such as neurones. The dendrimer may also be functionalised with further components to enhance cell targeting and/or to deliver further therapeutic agents to the cell.

Polyplexes can be used as vectors to deliver into cells the therapeutic RNA of the invention directly, or a nucleic acid molecule that expresses the therapeutic RNA of the invention. Polyplexes are complexes of (typically cationic) polymers with nucleic acids. The nucleic acid is usually a DNA molecule encoding a therapeutic RNA of the invention although RNA polyplexes (e.g. nanomicelles) can also be used. The polyplex may be functionalised with further components to enhance cell targeting and/or may deliver further therapeutic agents to the cell. Polyplexes may be targeted to particular cell types, such as neurones.

Liposomes can be used as vectors to deliver into cells the therapeutic RNA of the invention directly, or a nucleic acid molecule that expresses the therapeutic RNA of the invention. Liposomes may be targeted to particular cell types, such as neurones. Liposomes are spherical vesicles, having at least one lipid bilayer, which can be used for drug delivery. The liposome may be multilamellar or unilamellar. Liposomes designed to deliver the therapeutic RNA directly to specific cells such as neurones may be used. The liposome may be functionalised with further components to enhance cell targeting and/or to deliver further therapeutic agents to the cell.

Micelles or lipoplexes can be used as vectors to deliver into cells the therapeutic RNA of the invention directly, or a nucleic acid molecule that expresses the therapeutic RNA of the invention. Micelles are supramolecular assemblies of surfactant molecules, related to liposomes. However, the lipid layer of a micelle is a monolayer, not a lipid bilayer as in liposomes. Lipoplexes are supramolecular assemblies of cationic lipids and nucleic acids. Micelles or lipoplexes designed to deliver the therapeutic RNA directly to specific cells such as neurones may be used. The micelle or lipoplex may be functionalised with further components to enhance cell targeting and/or to deliver further therapeutic agents to the cell. The micelle or lipoplex may be targeted to particular cell types, such as neurones.

Cell-penetrating peptides also known as peptide transduction domains efficiently pass through cell membranes. Cell-penetrating peptides (CPPs)/peptide transduction domains (PTDs) themselves can be used as vectors, by associating the CPP/PTD with the therapeutic RNA of the invention, or with a nucleic acid molecule that expresses the therapeutic RNA of the invention. Alternatively, CPPs/PTDs can be used to functionalise another vector (e.g. as disclosed herein) to enhance the efficiency of cell entry of the vector. The CPP/PTD may be targeted to particular cell types, such as neurones.

Cell-based vectors may be used to deliver the therapeutic peptides of the invention. Cells may be taken from a donor (and the donor may be the subject of treatment with the cell-based vector comprising the vector of the invention). Cell-based vectors will comprise a nucleic acid molecule that expresses the therapeutic RNA of the invention. The cell-based vector will express the therapeutic RNA of the invention at the target site, e.g. in the brain. Expression of the therapeutic RNA of the invention by the cell-based vector may be under the control of a tissue-specific promotor or a promoter that can be activated or switched off by application of external stimuli.

### EXAMPLES

The following examples are set forth so as to provide those of ordinary skill in the art with a complete disclosure and description of how to practise the invention, and are not intended to limit the scope of the invention.

Identification and Characterisation of MIR-AS-lncRNAs that Repress Gene Translation

### Introduction

The present inventors identified MAPT-AS1, a lncRNA antisense to the human MAPT gene that encodes the microtubule-associated protein tau, which is associated with a large class of neurodegenerative diseases collectively known as tauopathies. The inventors have found that MAPT-AS1 inhibits MAPT translation, as evident from a shift of MAPT mRNA from actively translating polysomes to sub-polysomal fractions.

From human brain cDNA, the inventors cloned three alternative splicing isoforms (hereafter referred as tau-Natural Antisense Transcript t-NAT1, t-NAT2s, t-NAT21), associated with two alternative transcription start sites (TSS) (FIG. 1a) and supported by RNA-sequencing (RNA-seq) data from multiple human brain regions (FIG. 1b, FIG. 8) and by multiple CAGE tag clusters^{15,16} (FIG. 6e).

The inventors conclude that these MIR-lncRNAs may thus contribute to a new layer of translational regulation, with implications for homeostasis of neuronal proteins that are commonly disrupted in neurodegenerative diseases.

### MAPT-AS1 Transcript Identification and Characterisation

All MAPT-AS1 isoforms identified herein lack open reading frames (ORF) and are predicted to be bona-fide lncRNAs as denoted by their negative PhyloCSF scorer¹⁷ (FIGS. 6c, 7c). t-NAT1 starts at a proximal TSS and overlaps by 89 nucleotides with the MAPT 5′-UTR, upstream to the AUG start codon (FIG. 1a). t-NAT2s and t-NAT21 isoforms both start at a more downstream TSS, in an evolutionary conserved region spanning a large CpG island (FIG. 5e). The distal MAPT-AS1 3′-exon is shared by all isoforms and contains an embedded repetitive element of the mammalian-wide interspersed repeat (MIR) family, subclass MIRc, in inverse orientation (as defined by www.repeatmasker.org).

Alignment of each MAPT-AS1 isoform revealed a striking conservation of the lncRNA anatomy in non-human primates. t-NAT1 has perfect conservation of the splice junction in all primates (FIG. 6a, 6b). t-NAT21 alternative exons 1, 2 and 3 are highly conserved among great apes and Old World primates but not in New World primates (FIG. 7a, 7b), suggesting that t-NAT21 splicing pattern was only acquired after divergence of the New World monkeys from the other primate lineage, approximately 32-36 million years ago¹⁸. Notably, a highly conserved region of 62 nucleotides at the 3′-end of both isoforms of MAPT-AS1 is present in all primates, and shows homology to the CORE-SINE common to all MIR repeats¹⁹ (FIG. 6d, 7d), suggesting that this region likely represents a functional domain.

To characterize expression and localisation of MAPT-AS1 lncRNA, the inventors assessed the expression level of different splicing isoforms in a panel of 20 human tissues. All isoforms displayed a tissue-specific pattern of expression similar to MAPT, with highest levels in brain (FIG. 1c, FIG. 8). Analysis of RNA-Seq data from human post-mortem brain recently published²⁰, showed a positive correlation between MAPT-AS1 levels and MAPT mRNA (Pearson's correlation coefficient 0.7004; FIGS. 8, 14). Both MAPT-AS1 and MAPT mRNA showed parallel increases in expression during cortical neuronal differentiation of human induced pluripotent stem cells (hiPSC) (FIG. 1d, FIG. 9b). The inventors then assessed the intracellular localisation of t-NAT transcripts by single-molecule fluorescence RNA in situ hybridization (sm-FISH), employing 48 tiling antisense DNA probes covering all MAPT-AS1 isoforms and 26 probes complementary to MAPT mRNA (Extended Data Table 2). Mature MAPT mRNA and MAPT-AS1 were observed mainly in the cytoplasm, with few discrete nuclear spots, likely corresponding to transcription sites (FIG. 1e). Localisation in both nuclear and cytoplasmic compartments was confirmed by qRT-PCR after cellular fractionation (FIG. 2a).

Expression of neither t-NAT1 nor t-NAT21 isoforms of MAPT-AS1 in SH-SY5Y cells caused any significant change in endogenous MAPT mRNA (FIG. 2a, 2c, FIG. 10a). However, transient or stable expression of either t-NAT1 or t-NAT21 strongly and reproducibly reduced endogenous tau protein without affecting β-actin, TDP-43 or the neighboring gene IMP5 (FIG. 2b, 2f, 2g, FIG. 11). Conversely, siRNA-mediated knockdown of the endogenous MAPT-AS1 increased total tau protein in SH-SY5Y cells (FIG. 2d). These data indicate that MAPT-AS1 controls tau expression at a post-transcriptional level. Analysis of dephosphorylated tau showed that the MAPT-AS1-mediated decrease of tau protein is independent of tau phosphorylation (FIG. 2e, 2f).

### Determination of Functional Domains

To determine the MAPT-AS1 transcript regions required for tau repression, the inventors established several SH-SY5Y-derived cell lines stably overexpressing either full-length or targeted deletions of t-NAT1 and t-NAT21 isoforms. Full-length (FL) t-NAT1 or t-NAT21 transcripts consistently repressed tau protein levels when compared to empty-vector expressing control cells (Empty) (FIG. 2f, 2g). Deletion of the 5′-terminal exons (5′) or the 3′-terminal exon (3′) shared by t-NAT1 and t-NAT21 completely abolished this repression (FIG. 2e, 2f), suggesting that both 5′ and 3′ domains are necessary for MAPT-AS1 function.

Stable expression of variants of t-NAT1 5′-exon, with deletions either of the regions not overlapping with MAPT 5′-UTR (non) or of the overlapping region (over), or with the overlapping region placed in antisense orientation (flip), failed to reduce tau protein level (FIG. 2e, 2f). These data indicate that formation of the RNA duplex between MAPT-AS1 and MAPT is not sufficient for translational repression.

Surprisingly, cells stably expressing t-NAT1 or t-NAT21 lacking the MIR was unable to repress tau translation (ΔM2 or ΔM, FIG. 2f, 2g). In all cases no significant change in MAPT mRNA level was observed, demonstrating that MIR is critical for the translational repression by MAPT-AS1 (FIG. 9a). A partial deletion of the region spanning the MIR had no effect (ΔM1, partially deleted MIR FIG. 2f). A mutant with a flipped MIR sequence was equally unable to repress tau translation, thus proving the orientation-dependent activity of the MIR element (FIG. 10).

Tau translation is spatially and temporally controlled by the mTOR-p70S6K pathway via a 5′-terminal oligopyrimidine (TOP) sequence. This results in axonal accumulation of tau protein²¹, which contributes to the establishment of neuronal polarity. The complex folding of the MAPT 5′-UTR leads to two main domains that together function as an internal ribosome entry site (IRES)²², providing the cis-acting signals for an alternative mode of translational regulation. Apart from cap-dependent regulation, about 30% of tau translation proceeds through the IRES-mediated pathway, and the full-length structure of MAPT 5′-UTR (FIG. 3c) is necessary for maximal tau IRES activity²². It is not clear how tau switches between cap-dependent and cap-independent modes of translation, and which cellular factors are responsible for controlling tau IRES activity.

t-NAT1 5′-exon overlaps domain II of the MAPT IRES by 89 nucleotides where the 40S ribosomal subunit has been shown to bind²² (FIG. 3c). To test if MAPT-AS1 can affect tau translation through the IRES, the inventors cloned the full-length sequence of the human MAPT 5′-UTR (322 nt) into pRF, a dicistronic luciferase reporter vector to generate pRTF (FIG. 3a). The dicistronic construct contains a Renilla luciferase (Rluc) ORF and a Firefly luciferase (Fluc) ORF, separated by the MAPT 5′-UTR. Translation of Rluc occurs by a cap-dependent mechanism and is used to normalize for transfection efficiency. Translation of Fluc will not occur unless an IRES is present to initiate translation (pRF versus pRTF, FIG. 3b).

To exclude a possible involvement of other cis-regulatory elements in MAPT-AS1-mediated repression, the inventors tested involvement of the 3′-UTR. SH-SY5Y cells were co-transfected with a full-length or truncated MAPT 3′-UTR inserted downstream to a Fluc ORF together with either a pcDNA3.1 empty vector or wild-type MAPT-AS1 lncRNAs. No significant change in luciferase level was observed in the presence of either wild-type or different deletion mutants of t-NAT1 and t-NAT21 (FIG. 11), showing that MAPT-AS1 function does not require tau 3′-UTR.

Noting the role played by the embedded inverted MIR repeat in regulating the steady-state level of endogenous tau protein, the inventors tested if the MIR is directly involved in MAPT-AS1-mediated effects on tau IRES. As expected, SH-SY5Y cells transiently co-transfected with pRTF in presence of either t-NAT1 or t-NAT21 showed a reduced IRES activity compared to cells transfected with an empty vector (FIG. 3d). Surprisingly, cells co-transfected with either t-NAT1 or t-NAT21 with the entire MIR repeat region deleted could not repress tau IRES (FIG. 3d).

### Inhibition of Translation

To further validate that MAPT-AS1 inhibits tau translation, the inventors measured the enrichment of t-NAT isoforms, MAPT and β-actin (ACTB) mRNAs in polysome gradient fractions. t-NAT1 and t-NAT21 co-localised mainly with monosomes (80S) and disomes (FIG. 3f, FIG. 3f). Stable expression of either wild-type t-NAT1 or t-NAT21 significantly shifted the MAPT mRNA from heavy to lighter polysome fractions, confirming their role in translational repression (FIG. 3e; FIG. 12). Conversely, MAPT-AS1 without the embedded MIR repeat lead to increased engagement of the MAPT message with polysomes, resulting in a strong increase in tau translation (FIG. 3e, 3f).

These data indicate that the MIR repeat element modulates tau IRES activity, either by affecting its global RNA secondary/tertiary structure, or by preventing access of the 40S ribosomal subunit to the AUG starting codon. Alternatively the MIR repeat might mask short regions within domain I and II, which could potentially base-pair with 18S ribosomal RNA (rRNA)²².

Interestingly the inverted MIR element of MAPT-AS1 contains two close 7-mer motifs that are either complementary or identical to two conserved regions of the human 18S rRNA. As illustrated in FIG. 18 the first motif (CACCCAC, blue) is complementary to nucleotides 1318-1324 of the 18S rRNA at the basis of helix 33 (grey lines), and the other (CTGAGGC, red) is identical to nucleotides 905-911 in the stem 21esd6, which is part of the 18S rRNA expansion segment 6, only present in Eukaryotes. Both these MIR motifs could mediate MAPT IRES repression due to a direct competition for pairing with the 40S rRNA (FIG. 18). Interestingly as postulated on the basis of a large body of experimental observations, Vincent Mauro and Gerald Edelman proposed an interesting theory called “the ribosome filter hypothesis”, which predicts that the ribosome itself may act as a regulatory filter in determining translation rate for subsets of mRNAs, with which it can interact through direct rRNA-mRNA complementary sites, or mRNA-ribosomal protein interactions²³. The widespread occurrence of short complementarity regions within 5′-UTRs of human genes forming a well-defined tridimensional pattern on the 18S rRNA, that has been revealed for several distant species²⁴, points towards a role for rRNA-mRNA interactions in modulating the whole translation process. Within this context MIR retroelements embedded within antisense-lncRNAs, may directly and dynamically modulate these weak and transient interactions with 40S ribosomes, controlling the rate of protein synthesis for subsets of cellular mRNAs.

This is particularly relevant for all those mRNAs that are capable of an internal initiation mediated by IRES sequences, as most of these mRNAs are enriched for short motifs of complementarity to an “active region” (nt 812-1233) of the 18S rRNA in their 5′-UTRs, as it was shown in a recent genome-wide screening of human 5′-UTRs for their cap-independent translation activity²⁵. Noticeably, our kmer-enrichment analysis on all antisense MIR-lncRNAs, revealed that MIR-lncRNAs target genes are enriched for 7-mer motifs in their 5′-UTRs, matching the 18S rRNA “active region” as defined by Weingarten-Gabbay et al.²⁵ (See Table 1). Intriguingly 135 (27.7%) out of 487 human genes overlapping within their 5′-UTR with antisense MIR-lncRNAs, possess one or more motifs (7-mers) of 18S complementarity which are also found in the MIR element of their paired MIR-lncRNA. Strikingly these motifs cluster in specific areas within the 18S rRNA “active region” (FIG. 17, Table 1).

The 18S rRNA sequence is:

[SEQ ID NO: 18]

UACCUGGUUG AUCCUGCCAG UAGCAUAUGC UUGUCUCAAA
GAUUAAGCCA UGCAUGUCUG AGUACGCACG GCCGGUACAG
UGAAACUGCG AAUGGCUCAU UAAAUCAGUU AUGGUUCCUU
UGGUCGCUCG CUCCUCUCCU ACUUGGAUAA CUGUGGUAAU
UCUAGAGCUA AUACAUGCCG ACGGGCGCUG ACCCCCUUCG
CGGGGGGGAU GCGUGCAUUU AUCAGAUCAA AACCAACCCG
GUCAGCCCCU CUCCGGCCCC GGCCGGGGGG CGGGCGCCGG
CGGCUUUGGU GACUCUAGAU AACCUCGGGC CGAUCGCACG
CCCCCCGUGG CGGCGACGAC CCAUUCGAAC GUCUGCCCUA
UCAACUUUCG AUGGUAGUCG CCGUGCCUAC CAUGGUGACC
ACGGGUGACG GGGAAUCAGG GUUCGAUUCC GGAGAGGGAG
CCUGAGAAAC GGCUACCACA UCCAAGGAAG GCAGCAGGCG
CGCAAAUUAC CCACUCCCGA CCCGGGGAGG UAGUGACGAA
AAAUAACAAU ACAGGACUCU UUCGAGGCCC UGUAAUUGGA
AUGAGUCCAC UUUAAAUCCU UUAACGAGGA UCCAUUGGAG
GGCAAGUCUG GUGCCAGCAG CCGCGGUAAU UCCAGCUCCA
AUAGCGUAUA UUAAAGUUGC UGCAGUUAAA AAGCUCGUAG
UUGGAUCUUG GGAGCGGGCG GGCGGUCCGC CGCGAGGCGA
GCCACCGCCC GUCCCCGCCC CUUGCCUCUC GGCGCCCCCU
CGAUGCUCUU AGCUGAGUGU CCCGCGGGGC CCGAAGCGUU
UACUUUGAAA AAAUUAGAGU GUUCAAAGCA GGCCCGAGCC
GCCUGGAUAC CGCAGCUAGG AAUAAUGGAA UAGGACCGCG
GUUCUAUUUU GUUGGUUUUC GGAACUGAGG CCAUGAUUAA
GAGGGACGGC CGGGGGCAUU CGUAUUGCGC CGCUAGAGGU
GAAAUUCUUG GACCGGCGCA AGACGGACCA GAGCGAAAGC
AUUUGCCAAG AAUGUUUUCA UUAAUCAAGA ACGAAAGUCG
GAGGUUCGAA GACGAUCAGA UACCGUCGUA GUUCCGACCA
UAAACGAUGC CGACCGGCGA UGCGGCGGCG UUAUUCCCAU
GACCCGCCGG GCAGCUUCCG GGAAACCAAA GUCUUUGGGU
UCCGGGGGGA GUAUGGUUGC AAAGCUGAAA CUUAAAGGAA
UUGACGGAAG GGCACCACCA GGAGUGGAGC CUGCGGCUUA
AUUUGACUCA ACACGGGAAA CCUCACCCGG CCCGGACACG
GACAGGAUUG ACAGAUUGAU AGCUCUUUCU CGAUUCCGUG
GGUGGUGGUG CAUGGCCGUU CUUAGUUGGU GGAGCGAUUU
GUCUGGUUAA UUCCGAUAAC GAACGAGACU CUGGCAUGCU
AACUAGUUAC GCGACCCCCG AGCGGUCGGC GUCCCCCAAC
UUCUUAGAGG GACAAGUGGC GUUCAGCCAC CCGAGAUUGA
GCAAUAACAG GUCUGUGAUG CCCUUAGAUG UCCGGGGCUG
CACGCGCGCU ACACUGACUG GCUCAGCGUG UGCCUACCCU
ACGCCGGCAG GCGCGGGUAA CCCGUUGAAC CCCAUUCGUG
AUGGGGAUCG GGGAUUGCAA UUAUUCCCCA UGAACGAGGA
AUUCCCAGUA AGUGCGGGUC AUAAGCUUGC GUUGAUUAAG
UCCCUGCCCU UUGUACACAC CGCCCGUCGC UACUACCGAU
UGGAUGGUUU AGUGAGGCCC UCGGAUCGGC CCCGCCGGGG
UCGGCCCACG GCCCUGGCGG AGCGCUGAGA AGACGGUCGA
ACUUGACUAU CUAGAGGAAG UAAAAGUCGU AACAAGGUUU
CCGUAGGUGA ACCUGCGGAAGGAUCAUUA

The underlined part of the 18S rRNA sequence [SEQ ID NO: 19] corresponds with the “active region” as defined by Weingarten-Gabbay et al²⁵ and by Petrov et al³⁵.

Plasmid-based expression of the non-coding RNAs, we observed a strong reduction of tau protein levels with tNAT1 (NT1 wt in FIG. 19b, top panel) that was absent in controls or the tNAT1 variants with targeted deletions of the overlapping region and or a downstream exon (NT1D5 and NT1D3). tNAT2 (NT2 wt) expression showed a more modest reduction. With multiple independent clones of SH-SY5Y cells overexpression tNAT1 (NT1) and tNAT2 (NT2), we observed a much more striking reduction in tau protein levels for both NATs (see FIG. 19c and FIGS. 20a and b). FIG. 19c and FIG. 20a (top panel) show the effects of three independent clones overexpressing tNAT1 (NT1-1, NT1-2 and NT1-3) whereby tau protein levels are almost completely eliminated compared to empty vector clones (V5) and clones expressing variants of tNAT1 with deletions or rearrangements of the 5′ exon of the tNAT1 that overlaps with the MAPT promoter or the distal 3′ region of tNAT1. This clearly demonstrates the role of tNAT1 as a strong repressor of MAPT gene expression at the translation level and the importance of the 5′ region of both NATs that overlap with the MAPT core promoter region (FIG. 19a).

### Other AS-lncRNAs

In order to find other AS-lncRNAs that could have a similar effect to the MAPT-AS1, we screened the GENCODE v19 annotations for transcripts containing a MIR. We calculated the MIR coverage within each transcript normalized to their lengths. All classes of MIR elements, present almost equally in both orientations, are enriched in lncRNAs as opposed to protein-coding mRNAs (FIG. 4a). This is in line with a more general enrichment of TE in lncRNAs as was recently reported²⁶. The inventors then assessed whether other MIR-containing lncRNAs could post-transcriptionally regulate expression of their protein-coding targets.

The GENCODE v19 annotations were bioinformatically screened for AS-lncRNAs containing one or more embedded MIR elements. Considering the high degree of conservation of the CORE-SINE in all MIR subclasses¹⁹, we included lncRNAs containing other MIR classes (MIR, MIR3, MIRb, MIRc). Having observed that flipping of the MIR element leads to an opposite effect on target gene regulation (Extended Data, FIG. 7), we included lncRNAs with embedded MIR elements in both orientations. 5.63% of AS-lncRNAs contain an embedded MIR-repeat. We identified 1197 such MIR-lncRNAs, 40.69% of which overlap with the 5′-UTR, 32.50% overlap with the 3′-UTR and 26.81% with coding sequences (CDS) (FIG. 4b).

Interestingly, a Gene Ontology (GO)-term enrichment analysis using Enrichr²⁷ revealed that the region of overlap is associated with genes enriched in different cellular components and disease-linked loci. MIR-lncRNAs with overlap in the 5′-UTR are significantly enriched for genes expressed in the brain that are associated with dementia, Parkinson's disease or amyotrophic lateral sclerosis and localise mainly to axonal and neuronal projection membrane compartments (FIG. 4c, FIG. 4f).

### Experimental Validation in Other Genes

To further confirm that embedded MIR repeats within AS-lncRNAs may similarly repress translation of other protein-coding genes, we experimentally validated one such MIR-lncRNA overlapping with the first 5′-exon of the human gene encoding for phospholipase c gamma 1 (PLCG1) (FIG. 4d). SH-SY5Y cells expressing a full-length antisense MIR-lncRNA (PLCG1-AS FL) show a robust reduction of PLCG1 protein (FIG. 4e). Conversely cells expressing an antisense-lncRNA deleted of the inverted MIR repeat express a normal level of endogenous PLCG1 protein (FIG. 4e). Surprisingly, protein-coding genes paired with antisense MIR-lncRNAs have significantly more structured 5′-UTRs and 3′-UTRs (FIG. 15a, 15b). Finally, using the NetworkAnalyst tool²⁸, we observed that the majority of antisense MIR-lncRNA target coding genes belongs to a wide protein-protein interaction network, significantly enriched for genes linked to neurodegenerative diseases (adjusted P value 1.63×10⁻⁸, FIG. 4f; FIG. 15) and genes expressed in immune system (FIG. 16).

Furthermore genes overlapping in 5′-UTR with antisense MIR-lncRNAs are significantly more expressed in brain than genes overlapping along 3′-UTR or CDS as shown by the RNA-seq data from human post-mortem brains (FIG. 4g). These data uncover a novel role for an entire class of transposable elements (TE)-derived lcnRNAs, and suggest that MIR repeat elements might represent a widespread cis-regulatory signal employed by lncRNAs to orchestrate translational regulation of many genes involved in neuronal proteins homeostasis, frequently affected in neurodegenerative diseases. This novel function for MIR repeats in the cytoplasm adds to their previously documented function as transcriptional enhancers or insulators in the nucleus^{29,30}.

### Materials and Methods

### Plasmids

cDNA sequence of human antisense t-NAT1 and t-NAT21 were amplified from a sample of human brain total RNA (Clontech, 636530) with the primers NT1-5′F, NT1-3′R and TOPO2-F, TOPO2-R respectively:

NT1 5′F (BamHI)
GGCggatccGCCCCAGTCTGCGGAGAGG
NT1 3′R (XhoI)
GGCctcgagGCTGTGTCAATGTGGGCTTC
TOPO2-F (EcoRV)
GGCgatatcCCATGAAGTGGGTGTATT
TOPO2-R (BamHI)
GGCggatccCGAGTCCGTCCACATCGCCA

The antisense t-NAT1 5′deletion mutant (Δ5′) was generated by PCR using the oligonucleotides forward NT1Δ5-BamHI and reverse NT1Δ5-XhoI. PCR fragment was cloned directionally in the unique BamHI and XhoI sites in pcDNA3.1V5 (Invitrogen). Similarly the antisense t-NAT21 5′ deletion mutant (Δ5′) was generated by PCR using the forward NT245-BamHI and reverse NT245-XhoI primers and cloned in the same sites in pcDNA3.1V5.

The antisense t-NAT1 3′ deletion mutant (Δ3′) was generated by PCR using the forward NT1Δ3-BamHI and reverse NT1Δ3-XhoI primers and cloned in the unique BamHI and XhoI sites in pcDNA3.1V5. Similarly the antisense t-NAT21 3′ deletion mutant (Δ3′) was generated by PCR using the forward NT2Δ3-BamHI and reverse NT2Δ3-XhoI primers and cloned in the same sites in pcDNA3.1V5.

The antisense t-NAT1 (4M1) (partial ΔMir, 386-433) mutant was obtained by cloning of a PCR fragment amplified using the primers (NT1Δ3-BamHI and NT1Δmir1-XhoI) into the BamHI-XhoI sites of pcDNA3.1V5.

The antisense t-NAT1 (4M2) (total ΔMir, 386-449) mutant was obtained by cloning of a PCR fragment amplified using the primers (NT1Δ3-BamHI and NT1Δmir2-XhoI) into the BamHI-XhoI sites of pcDNA3.1V5.

The antisense t-NAT21 (ΔM) (ΔMir, 498-532) mutant was obtained by cloning of a PCR fragment amplified using the primers (NT2Δ3-BamHI and NT2Δmir-XhoI) into the BamHI-XhoI sites of pcDNA3.1V5.

The antisense t-NAT1 (over) (S/AS overlapping region, 93-168) fragment was generated by direct ligation of in vitro annealed oligonucleotides, with reconstituted 5′-end overhangs, forward NT1overS and reverse NT1overAS (75 nt) onto BamHI and XhoI sites of pcDNA3.1V5. Similarly the antisense t-NAT1 (Flip) (S/AS overlapping region in a Flipped orientation, 168-93) fragment was generated by direct ligation of in vitro annealed oligonucleotides forward NT1overFlipS and reverse NT1overFlipAS (75 nt) onto BamHI and XhoI sites of pcDNA3.1V5.

The antisense t-NAT1 (non) (non-overlapping region, 4-93) mutant was obtained with a similar strategy to antisense t-NAT1 (over). Oligonucleotides forward NT1nonoverS and reverse NT1nonoverAS were annealed in vitro and directionally ligated onto BamHI and XhoI sites of pcDNA3.1V5.

The antisense t-NAT1 (Mflip) (MIR repeat flipped) mutant was obtained as a gene synthesis construct (GENEWIZ) and subcloned into pcDNA3.1V5 using BamHI and XhoI restriction sites.

Full-length antisense-PLCG1 lncRNA (ENST00000454626.1, 1,459 nt) was designed as a gene synthetic construct (GENEWIZ) and subcloned into pcDNA3.1V5 using BamHI and EcoRVrestriction enzymes. Similarly an antisense-PLCG1 lncRNA deleted of the inverted MIRb repeat in its third exon (antisense-PLCG1—ΔM, 1333 nt) was also generated by gene synthesis (GENEWIZ) subcloned into pcDNA3.1V5 using BamHI and EcoRV restriction enzymes.

B1b-MluI-F
GTCacgcgtGAGTGCGCGCGTG
B1b-MluI-F
GTCacgcgtGAGTGCGCGCGTG
B2-F-NheI
agtGCTAGCTGCCGCTGTTCGCCATCAG
B2-R-EcoRV
tagccGATATCACCCTCAGAATAAAAGCCAG
B1F-NruI
cctTCGCGACAAATGCTCTGCGATGTGTT
B1R-HindIII
cctAAGCTTGGACAGCGGATTTCAGATTC
NT1Δ5-BamHI
GAAggatccGAGTCAGAACAAGGACGG
NT1Δ5-XhoI
GAActcgagTTGCTGTGTCAATGTGGG
NT1Δ3-BamHI
GAAggatccAGTCTGCGGAGAGGGAG
NT1Δ3-XhoI
GAActcgagCTTCTGCCGCCGCCA
NT1overS:
gatccCTTCTGCCGCCGCCACCACAGCCACCTTCTCCTCCTCCGCTGTCC
TCTCCCGTCCTCGCCTCTGTCGACTATCAGc
NT1overAS:
gGAAGACGGCGGCGGTGGTGTCGGTGGAAGAGGAGGAGGCGACAGGA
GAGGGCAGGAGCGGAGACAGCTGATAGTCgagct
NT1overFlipS:
gatccCTGATAGTCGACAGAGGCGAGGACGGGAGAGGACAGCGGAGGA
GGAGAAGGTGGCTGTGGTGGCGGCGGCAGAAGc
NT1overFlipAS:
gGACTATCAGCTGTCTCCGCTCCTGCCCTCTCCTGTCGCCTCCTCCTC
TTCCACCGACACCACCGCCGCCGTCTTCgagct
NT1nonoverS:
gatccAGTCTGCGGAGAGGGAGGGCGAGGGGCGGCGGCGCAGGGGTGC
ACAGAGGCGGACGGCGAGGCAGATTTCGGAGCCGCGGCGCTTACc
NT1nonoverAS:
gTCAGACGCCTCTCCCTCCCGCTCCCCGCCGCCGCGTCCCCACGTGTC
TCCGCCTGCCGCTCCGTCTAAAGCCTCGGCGCCGCGAATGgagct
NT2Δ5F-BamHI
GAAggatccGAGTCAGAACAAGGACGG
NT2Δ5R-XhoI
GAActcgagCCATGAAGTGGGTGTATTAAGG
NT2Δ3F-BamHI
GAAggatccCCGAGTCCGTCCACAT
NT2Δ3R-XhoI
GAActcgagCTGCAGGGGAAAGGTG
NT1Δmir1-XhoI
GAActcgagTTGCTGTGTCAATGTGGGGGTAACGTTAAGTGGCTATA
NT1Δmir2-XhoI
GAActcgagGGTAACGTTAAGTGGCTATA
NT2ΔmirR-XhoI
GAActcgagGGTAACGTTAAGTGGCTATAACAAAGAGACTC
B1LucF-BamHI
GAAggatccCTTGGCAATCCGGTACTGTT
B1LucR-HindIII
GAAaagcttCCGTCTTCGAGTGGGTAGAA
B1LucF-HindIII
GAAaagcttCTTGGCAATCCGGTACTGTT
B1LucR-BamHI
GAAggatccCCGTCTTCGAGTGGGTAGAA
CP-F
GAGCTCCAAATGCTCTGCGATGTGTT
CP-R
GCTAGCGGACAGCGGATTTCAGATTC
NP-F
gaGCTAGCTGCCGCTGTTCGCCATCAG
NP-R
gtGCTAGCACCCTCAGAATAAAAGCCAG
Fr1-F
GAGGTCCCTGGGGCGGTCAATAA
Fr1-R
AAGCTTAGGCAGTGATTGGGCTCTC
Fr2-F
GAGCTCGTAGGGGGCTGAGTTGAG
Fr2-R
AAGCTTACCAGAAGTGGCAGAATTGG
Fr3-F
GAGCTCCAGACTGGGTTCCTCTCCAA
Fr3-R
AAGCTTGCCAGCATCACAAAGAAG
RVp3-F
TAGCAAAATAGGCTGTCCCC
Luc-R
ATGTGCGTCGGTAAAGGCG
pRTF-EcoRI
GGTTTgaattcGGACGGCCGAGCGGCA
pRTF-NcoI
TAAccatggCCTGGTTCAAAGTTCACCTGATAGTCGACAGAGGCGAG
pRTFDover-EcoRI
GGTTTgaattcCCTTCTGCCGCCGCCAC
pRTFDover-NcoI
TAAccatggTGGGCGGTGGCAGCG
pRTF-mTOP
CCGCGCTGCCCGCCCaaTaaaaTGGGGAGGCTCGCGTTCC
pRTFDover-PvuII
GGTTTcagctgCCTTCTGCCGCCGCCAC
pRFseq-F
GAAGGTGCCAAGAAGTTTCCTAA

### Cells

SH-SY5Y and SK-N-F1 human neuroblastoma cells were obtained from ATCC. Cells were seeded in 75-cm² flasks in complete medium containing 44% Minimum Essential Medium Eagle (MEME), 44% Ham's nutrient mixture (F12), 10% fetal bovine serum (Sigma) supplemented with 1% non essential aminoacids (Sigma), 1% L-glutamine (Sigma), 0.1% Amphotericin B (Gibco), penicillin (50 units ml⁻¹) and streptomycin (50units ml⁻¹), and maintained at 37° C. with 5% CO₂. For experiments, 60% confluent cells were plated in 6-well plates (VWR), grown overnight before transfection and harvested 48 hours post-transfection. Transient transfections were done with TransFast (Promega).

For establishing the stable cell lines (Empty pcDNA 3.1, t-NAT1 FL, t-NAT1Δ5′, t-NAT1Δ3′, t-NAT1over, t-NAT1Flip, t-NAT1non, t-NAT1ΔM2, t-NAT2 FL, t-NAT2Δ5′, t-NAT2Δ3′, t-NAT2ΔM), SH-SY5Y cells were seeded in 10-cm Petri dishes and transfected with TransFast (Promega) and 7.5 μg plasmid DNA according to the manufacturer's instruction. Stable clones were selected by 500 μM G418 sulfate (345810, Millipore). For each type of stable cell line, at least 6 independent clones were isolated using glass cloning cylinders (C1059, Sigma), expanded in 6-well plates and screened individually by Western Blot and qRT-PCR.

Induced Pluripotent Stem Cells (iPSC) and Cortical Neuron Cultures

The control induced pluripotent stem cells (iPSCs) used in this study have been previously generated by retroviral expression of c-Myc, Oct4, Klf4 and Sox2³¹. IPSCs were grown under feeder-free conditions on Geltrex-coated plates in Essential 8 medium (Thermo Scientific). The medium was replaced daily and iPSCs were passaged every 5-6 days with 0.5 mM EDTA (Thermo Scientific).

iPSCs were subsequently differentiated into cortical neurons, as previously described (Sposito et al. 2015), using dual SMAD inhibition followed by in vitro neurogenesis. Briefly, iPSCs were plated at 100% confluency and the media was switched to neural induction media (1:1 mixture of N-2 and B-27-containing media supplemented with the SMAD inhibitors Dorsomorphin and SB431452 (Tocris). N-2 medium consists of DMEM/F-12 GlutaMAX, 1×N⁻¹ insulin, 1 mM 1-amino acids, β-mercaptoethanol, 50 U ml⁻¹ penicillin and 50 mg ml⁻¹ streptomycin. B-27 medium consists of Neurobasal, 1×B-27, 200 mM 1-glutamine, 50 U ml⁻¹ penicillin and 50 mg ml⁻¹ streptomycin) (Thermo Scientific). At the end of the 10-day induction period, the converted neuroepithelium was replated onto laminin-coated plates using dispase (Thermo Scientific) and maintained in a 1:1 mix of the described N-2 and B-27 media which was replaced every 2-3 days. During the stage of neurogenesis around days 25-35, neuronal precursors were passaged further with accutase (Thermo Scientific) and plated for the final time at day 35 onto poly-ornithine and laminin coated plates (Sigma).

### Double Immunofluorescence

Neurons were fixed in 4% PFA for 25 minutes at room temperature, followed by 10 min permeabilisation in 0.25% Triton-X100/PBS and 30 min blocking in 3% BSA and 0.1% Triton-X100/PBS. Neurons were incubated with primary antibody overnight at 4° C. (Table). The following primary antibodies were used: anti-PAX6 (Covance, Rabbit, 1:500); anti-OTX2 (Millipore, Rabbit, 1:500); anti-Ki67 (BD, Mouse, 1:500); anti-TBR1 (Abcam, Rabbit, 1:300); anti-SATB2 (Abcam, Mouse, 1:100); anti-BRN2 (SantaCruz, Goat, 1:400); anti-Tujl (βIII-tubulin) (Covance, Mouse and Rabbbit, 1:2000). Incubation with secondary Alexa Fluor 488 and 568-conjugated secondary antibodies, (Thermo Scientific) both diluted 1:200 in 3% BSA in 0.1% Triton-X100/PBS, was performed for 1 h at room temperature. Nuclei were stained using DAPI and cells were mounted on slides with Prolong Gold Antifade Reagent (Thermo Scientific). Images were obtained using a Zeiss LSM 710 microscope.

### Splinkerette PCR

Sites of integration of individual clones of stable cell lines were determined following the method described in Potter and Luo (2010)³².

### RNA-Seq Library Preparation and Sequencing

Brain samples for analysis were provided by the Medical Research Council Sudden Death Brain and Tissue Bank (Edinburgh, UK). All four individuals sampled were of European descent, neurologically normal during life and confirmed to be neuropathologically normal by a consultant neuropathologist using histology performed on sections prepared from paraffin-embedded tissue blocks.

Twelve central nervous system regions were sampled from each individual. The regions studied were: cerebellar cortex, frontal cortex, temporal cortex, occipital cortex, hippocampus, the inferior olivary nucleus (sub-dissected from the medulla), putamen, substantia nigra, thalamus, hypothalamus, intralobular white matter and cervical spinal cord. RNA was extracted using Qiagen tissue kits (Qiagen, US), and quality controlled as detailed previously²⁰. cDNA Libraries were prepared by the UK Brain Expression Consortium in conjunction with AROS Applied Biotechnology A/S (Aarhus, Denmark).

Reverse transcription in this protocol is carried out using both oligo dT and random primers. This allowed total RNA profile patterns to be assessed with the latter and locations of splicing to be inferred.

qRT-PCR

Total RNA was extracted from cells and human post-mortem brain tissue samples (temporal cortex, occipital cortex, caudate) using Trizol reagent (Invitrogen) according to the manufacturer's instruction. A panel of RNA from 20 different normal human tissues (each consisting of pools of three tissue donors with full documentation on age, sex, race, cause of death) was obtained from Ambion (AM6000). The amplified transcripts were quantified using the comparative Ct method and the differences in gene expression were presented as normalized fold expression (ΔΔCt). All of the experiments were performed in duplicate. A heat map graphical representation of rescaled normalized fold expression (ΔΔCt/ΔΔCtₘₐₓ) was obtained by using Matrix2png (http://www.chibi.ubc.ca/matrix2png/).

Two-Colour Single-Molecule RNA Fluorescent In Situ Hybridization (sm-FISH)

DNA tiling probes complementary to 3 alternative splicing isoforms of human t-NAT transcripts (t-NAT1, t-NAT2s, t-NAT21), designed by using Stellaris Probe designer, were labeled at 3′-end with the fluorescent dye Quasar 670. Other DNA tiling probes complementary to the exons of human MAPT transcript (NM_005910) were labeled at the 3′-end with the dye Quasar 570. All FISH probes were 19 to 20 bp long, designed with a stringency factor 2, checked using BLAST, and obtained from Biosearch technologies. Fluorescent in situ hybridization was performed as previously described⁴.

siRNA Knockdown

SH-SY5Y cells were seeded at 70% of confluence in 6-well plates, and after 24 h were transfected with 75 μl of 2 μM siRNAs, using RNAiMax (Invitrogen) transfection reagent following manufacturer's instructions. After 48 h cells were harvested for protein and RNA extraction. Three independent pools of siRNAs (Ambion) were used to target different MAPT-AS1 exons as follows:

siNT1nover (S, CGGCGAGGCAGAUUUCGGAtt; AS, UCCGAAAUCUGCCUCGCCGtc); siNT2nover (S, GCCGCCGAGUCCGUCCACAtt; AS, UGUGGACGGACUCGGCGGCcg); siEx4-n268302 (S, AGGACAAUGUCCUAAGGAAtt; AS, UUCCUUAGGACAUUGUCCUcc); siEx4-n268298 (S, GAUUUGUCAUGAGUCUCUUtt; AS, AAGAGACUCAUGACAAAUCaa). A scrambled sequence #2 was used as negative control. Pre-designed and custom-designed were LNA-modified as Silencer® Select siRNAs (Ambion).

### Western Blot

Cells were lysed in RIPA lysis buffer supplemented with complete EDTA-free protease inhibitor cocktail (Roche). Protein lysate concentrations were measured by the BCA protein assay (Bio-Rad). Immunoblotting was performed with the following primary antibodies: anti-MAPT (T-1308-1, rPeptide, and A0024, and DAKO rabbit polyclonal), anti-β-actin (Δ2228, Sigma), anti-IMP5 polyclonal antibody (12664-1-AP, Proteintech) and anti-TDP43 (10782-2-AP, Proteintech), anti-PLCG1 (D9H10, rabbit monoclonal, Cell Signaling). Secondary antibodies were as follows: IRDye-800CW or IRDye-680CW conjugated goat anti-rabbit, donkey anti-mouse, donkey anti-rabbit, goat anti-mouse or anti-goat IgG (Li-COR Bioscience). Signals were digitally acquired by using an Odyssey infrared scanner (Li-COR Bioscience) and quantified using Fiji version 2.0.0-rc-39/1.50d (http://fiji.sc/Fiji).

### Cellular Fractionation

Nucleo-cytoplasmic fractionation was performed using Nucleo-Cytoplasmic separation kit (Norgen) according to the manufacturer's instruction. RNA was eluted and treated with DNase I (Roche). RNA concentrations were measured by NanoDrop spectrophotometer. The purity of the cytoplasmic fraction was confirmed by qRT-PCR on pre-ribosomal RNA.

### Luciferase Reporter Vectors

Firefly luciferase reporter plasmids were constructed by inserting the human MAPT core promoter (CP, 1,342 bp) amplified using the primers (CP-F GAGCTCCAAATGCTCTGCGATGTGTT, CP-R GCTAGCGGACAGCGGATTTCAGATTC) between the SacI and NheI sites into pGL4.10 vector (Promega) to create pGL4-CP vector. A 901 bp fragment of genomic DNA spanning the t-NAT promoter (NP) was amplified using the primers (NP-F gaGCTAGCTGCCGCTGTTCGCCATCAG, NP-R gtGCTAGCACCCTCAGAATAAAAGCCAG) and inserted into NheI site either of pGL4-CP or pGL4.10 vectors to create pGL4-CNP and pGL4-NP respectively. The full-length 322 bp-long human MAPT 5′-UTR was amplified with primers (pRTF-EcoRI, pRTF-NcoI) and ligated onto EcoRI and NcoI sites of the pRF vector (a kind gift from Prof. Anne Willis, Leicester University, UK) to create the pRTF vector. A fragment of MAPT 5′UTR devoid of t-NAT overlapping region was amplified using the primers (pRTF-EcoRI, pRTFDover-NcoI) and inserted between same sites into pRF, to generate the pRTF-Delta vector. pRTFover vector was constructed in the same way using the primers (pRTF-Dover-EcoRI, pRTF-NcoI). A pRhcvF, used as a positive control viral IRES, was a kind gift of Prof. Anne E. Willis and was constructed as described previously⁵. Mutant reporter plasmids were created using the QuickChange lightning multi site-directed mutagenesis kit (Agilent) according to the manufacturer's instructions. The following mutagenic oligonucleotides (pRTF-mTOP) were annealed to the pRTF vector, extended by PCR, and the parental methylated plasmid DNA was digested with Dpnl enzyme to obtain the correspondent mutant dicistronic luciferase vector. The full-legth human MAPT 3′UTR and 3 partially overlapping fragments were amplified from brain cDNA with the primers (Fr1-F, Fr1-R, Fr2-F, Fr2-R, Fr3-F, Fr3-R) and cloned individually into SacI and HindIII sites of pMIR-REPORT vector (Invitrogen).

### Dual Luciferase Reporter Assay

SH-SY5Y cells or t-NAT-stably expressing cells were seeded in Greiner 96-well plates overnight and then co-transfected using TransFast (Promega) with the dicistronic reporter vector pRF, pRhcvF, pRTF or pRTF deletion mutants and either a pcDNA3.1 empty vector or each of the t-NAT expression vectors. 48 h after transfection cap-dependent translation (Renilla luciferase activity) and IRES-mediated translation (Firefly luciferase activity) were measured with the DualGlo Luciferase Assay kit (Promega) according to the manufacturer's instructions. Firefly to Renilla ratios were normalized to a common pMIR-Report vector used to account for transfection efficiency in each experiment and results are represented as mean±s.d. Experiments were done in triplicate.

### Polysomal Fractionation

1×10⁶ cells were seeded in two 10 cm² dishes and collected for polysomal fractionation after 48 h. All the experiments were run in biological triplicate. Cells were incubated for 4 min with 100 μg/ml cycloheximide at 37° C. to block translational elongation. Cells were washed with PBS supplemented with 10 μg/ml cycloheximide, scraped into 300 μl lysis buffer (10 mM NaCl, 10 mM MgCl2, 10 mM Tris-HCl, pH 7.5, 1% Triton X-100, 1% sodium deoxycholate, 0.2 U/μl RNase inhibitor [Fermentas Burlington, Calif.], 100 μg/ml cycloheximide and 1 mM DTT) and transferred to a microfuge tube. Nuclei and cellular debris were removed by centrifugation at 13,000 g for 5 min at 4° C. The supernatant was layered on a linear sucrose gradient (15-50% sucrose (w/v) in 30 mM Tris-HCl at pH 7.5, 100 mM NaCl, 10 mM MgCl₂) and centrifuged in a SW41Ti rotor (Beckman Coulter, Indianapolis, Ind.) at 180,000 g for 100 min at 4° C. Ultracentrifugation separates polysomes by the sedimentation coefficient of macromolecules: gradients are then fractionated and mRNAs in active translation (polysome-containing fractions) are separated from untranslated mRNAs (subpolysomal fractions). Fractions of 1 ml volume were collected with continuous absorbance monitoring at 254 nm. As controls, cell lysates were treated with 50 mM EDTA on ice for 10 min before gradient loading.

qRT-PCR of Polysomal Fractions and Statistical Analysis

Total RNA was extracted from each polysomal fraction using 1 ml of Trizol (Invitrogen) following manufacturer's instructions. After DNAse I treatment, equal volumes of RNA were retro-transcribed in the presence of an equimolar mixture of oligo dT and random hexamer, using Super Script III (Invitrogen). For the statistics of polysome fractionation qRT-PCR analyses, the raw Ct value for each of the individual fractions was transformed to 2^{-Ct} and normalized to the sum total for all fractions, generating a percentage of total transcript within each fraction. Each fraction's values were aggregated into different categories corresponding to different phases of polysome assembly on a total RNA absorbance curve. For qRT-PCR analysis we followed a previously published method (Matthew L. Kraushar et al. PNAS 2014). Briefly: fractions 1 and 2 were summed into “40S-60S”; fractions 3 and 4 were summed into “80S”; fractions 5-7 were summed into “light”; fractions 8-10 were summed into “medium” and fractions 11-13 were summed into “heavy”-corresponding to peaks on total RNA absorbance curves monitored during fractionation. For significance testing of qRT-PCR data, t tests were conducted between Empty vector and t-NAT-expressing cells in each category, with p<0.05 considered significant, s.d. is shown as error bars in figures.

### Bioinformatic Analysis

Bedtools v2.2, Python 2.7.5 and R v.3.1.1 were used extensively during analysis. All plots were produced using R package ggplot2 and data processing was done using dplyr and tidyr.

Combining all Transcript Exons into Single Gene Annotations

For each gene a single non-overlapping list of exons was created, by merging exons from all transcripts. Each exon was defined as either 5′UTR, 3′UTR or CDS using GENCODE v19 comprehensive (hg19 build) annotations (http://www.gencodegenes.org/releases/19.html). All exons with multiple annotations were preferentially defined as either 5′UTR or 3′UTR. All further analysis utilized this annotation.

Identifying Overlapping lncRNA—Protein-Coding Gene S-AS Pairs and Defining Gene Groups

For the identification of additional translational repressor candidates, we searched for GENCODE v19 transcripts that were non-coding RNAs and overlap the 5′ UTR, CDS or 3′ UTR of coding transcripts in a head-to-head configuration. All protein-coding genes were intersected with lncRNAs from GENCODE v19 and these lncRNAs were then checked for overlaps with MIR elements from RepeatMasker (repeatmasker.org). These intersections were used to create the following groups:
All protein coding genes
Protein coding genes without lncRNA overlap
Protein coding genes with lncRNA overlap
Protein coding genes that overlap lncRNA that include MIR elements
Protein coding genes that overlap lncRNA that do not include MIR elements

Various analyses were applied to these groups, namely:

### Calculating an Estimate of Gene Feature Length Relative to Exon Number

From the non-overlapping exon annotation we were able to calculate a normalized number of exons per gene region (5′UTR, 3′UTR or CDS) by dividing the total number of exons within all gene transcripts by the sum of transcripts. This value was used to divide by the total length of gene region to estimate the length of feature compared to the number of exons. A one-way ANOVA followed by Dunnett's multiple comparison test was performed on the different gene groups to determine if the distributions between groups were significantly different.

### Predicting Secondary Structures for Protein-Coding Gene UTRs

For each gene the longest 5′UTR and 3′UTR were selected as representative for the gene. RNAfold v2.1.9 from the ViennaRNA Package was used to predict the minimum free energy (mfe) of the secondary structure (kcal/mol). A one-way ANOVA followed by Dunnett's multiple comparison test was performed on the different gene groups to determine if the distributions between groups were significantly different.

### Calculating the MIR Element Nucleotide Overlap Per Transcript

The non-overlapping length of each gene feature or lncRNA transcript was divided by the number of base pairs overlapping a RepeatMasker defined MIR repeat element. This provided an indication of relative abundance of MIR elements across the human transcriptome.

### Gene Expression Analysis of Postmortem Brain Tissue

Post mortem, total RNA sequence data was aligned using the STAR aligner v2.3 with default settings and GENCODE annotations. Gene counts and FPKM values were calculated based on the non-overlapping annotation for each gene using Bedtools v2.2 and custom python scripts. All regions were merged into a single mean value to describe whole brain expression of protein-coding genes.

### Statistical Analysis

Statistical analyses were performed using GraphPad PRISM5. A paired two-tailed Student's t-test was performed when comparing two categories. When more than two groups were compared, one-way ANOVA followed by a Dunnett's multiple comparison test was used. Results are mean (n≥3) ±standard deviation (s.d.).

Stable expression in cell lines can be used to characterize the effect of overexpression of the other MIR-lncRNAs, identified as described herein.

### Multialignment of MIR Elements

Multialignment of MIR elements of different subfamilies, here shown in inverse orientation with the CORE-SINE underlined.

CLUSTAL format alignment by MAFFT (v7.293)

| | |
|---|---|
| MIRc | aataataataaccccttacatttgtatagcactttacagtttacaaagcgct-------- |
| MIRb | aat----aatagagctaccatttattgagcgc-ttactgtgtgccaggcactgtgctaag |
| MIR | --------taataaccaacatttattgagcgc-ttactatgtgccaggcactgttctaag |
| MIR3 | ------------------------------------------------------------t |
| MIRc | ---ttcacatacattatctcat------ttgatcctcacaacaaccctgtgaggtaggca |
| MIRb | cgctttacatgcattatctcat------ttaatcctcacaacaaccctgcgaggtagg-- |
| MIR | cgctttacatgtattaactcat------ttaatcctcacaacaaccctatgaggtaggta |
| MIR3 | ttttttagaatcatagaatcatagaatgttagagctggaagggaccttagagatcatcta |
| | **.* * .** . **** **.. ** . *. .***.*. ... .* |
| MIRc | gggcaggtattattatccccattttacagatgagg-aaactgaggctcagagaggttaag |
| MIRb | -------tattattatccccattttacagatgagg-aaactgaggctcagagaggttaag |
| MIR | ------ctattattatccccattttacagatgagg-aaactgaggcacagagaggttaag |
| MIR3 | -------gtccaacccnctcattttacagatgaggaaaactgaggcccagagaggtgaag |
| | ..* . . *.**************** ********** ********* *** |
| MIRc | tgacttgcccaaggtcacacagctagtaagtggcagagccaggactcgaacccaggtctt |
| MIRb | tgacttgcccaaggtcacacagctagtaagtggcagagccaggattcgaacccaggtctg |
| MIR | taacttgcccaaggtcacacagctagtaagtggcagagccgggattcgaacccaggca-g |
| MIR3 | tgacttgcccaaggtcacacagctagttagtggcagagctaggactagaacccaggtc-t |
| | *.************************* ***********..***.* *********. |
| MIRc | cctgactccaagtccag-tgctctt-tccactgcaccacactgcctcg---- |
| MIRb | cctgactccaaagcccg-tgctctt-tccactgcaccacgctgcccctctg- |
| MIR | tctggctccagagtccg-tgctcttaaccactatgctatactgt-------- |
| MIR3 | cctgactcctagtccagttgttctt-tccactataccatactgcttccagaa |
| | .***.**** .. .* * **.**** *****...*.*..***. |

TABLE 1
Antisense orientation MIR-lncRNA genes

| | | | | | Kmer | | kmer | | | | | Known |
|---|---|---|---|---|---|---|---|---|---|---|---|---|
| | | | | Orientation | from | kmer in | in | | | kmer | Known | motif |
| | | target | exon | of | MIR- | UTR | UTR | kmer 18s | kmer 18s | 18s | motif | Adjusted |
| lnc | target_gene | gene name | type | repeat | lncRNA | posn | orientatn | posn | status | orientatn | pvalue | pval |
| LENSG00000234775.1 | ENSG00000163485 | ADORA1 | 5P | Anti | TTCCTTT | [551, | [‘F’, | [114, | [‘I’, | [‘F’, | 2.32E−80 | 9.53E−78 |
| | | | | | | 201] | ‘R’] | 1193] | ‘A’] | ‘R’] | | |
| LENSG00000234775.1 | ENSG00000163485 | ADORA1 | 5P | Anti | ATTCCTT | [202] | [‘R’] | [1194] | [‘A’] | [‘R’] | 3.15E−43 | 4.31E−41 |
| LENSG00000234775.1 | ENSG00000163485 | ADORA1 | 5P | Anti | TATTCCT | [203] | [‘R’] | [857] | [‘A’] | [‘R’] | 5.34E−43 | 5.49E−41 |
| LENSG00000244968.2 | ENSG00000113594 | LIFR | 5P | Anti | TCTTAAT | [177] | [‘R’] | [915] | [‘A’] | [‘R’] | 1.44E−38 | 8.44E−37 |
| LENSG00000117543.15 | ENSG00000269175 | AC093157.1 | 5P | Anti | ATGCTTT | [779, | [‘F’, | [995] | [‘A’] | [‘R’] | 1.12E−35 | 5.13E−34 |
| | | | | | | 204] | ‘R’] | | | | | |
| LENSG00000257913.1 | ENSG00000181929 | PRKAG1 | 5P | Same | ATGCTTT | [225, | [‘F’, | [995] | [‘A’] | [‘R’] | 1.12E−35 | 5.13E−34 |
| | | | | | | 249] | ‘R’] | | | | | |
| LENSG00000177640.11 | ENSG00000107560 | RAB11FIP2 | 5P | Anti | TTTGGTT | [539] | [‘R’] | [1143] | [‘A’] | [‘R’] | 8.19E−31 | 2.81E−29 |
| LENSG00000271122.1 | ENSG00000122557 | HERPUD2 | 5P | Same | TTTGGTT | [440] | [‘R’] | [1143] | [‘A’] | [‘R’] | 8.19E−31 | 2.81E−29 |
| LENSG00000177640.11 | ENSG00000107560 | RAB11FIP2 | 5P | Same | TTTGGTT | [539] | [‘R’] | [1143] | [‘A’] | [‘R’] | 8.19E−31 | 2.81E−29 |
| LENSG00000267163.1 | ENSG00000267680 | ZNF224 | 5P | Anti | CTGCTTT | [394] | [‘F’] | [824] | [‘A’] | [‘R’] | 1.88E−30 | 5.95E−29 |
| LENSG00000267163.1 | ENSG00000267680 | ZNF224 | 5P | Same | CTGCTTT | [394] | [‘F’] | [824] | [‘A’] | [‘R’] | 1.88E−30 | 5.95E−29 |
| LENSG00000233251.3 | ENSG00000055813 | CCDC85A | 5P | Anti | TTCCATT | [190] | [‘R’] | [863] | [‘A’] | [‘R’] | 9.26E−27 | 2.38E−25 |
| LENSG00000227218.3 | ENSG00000136908 | DPM2 | 5P | Anti | TTCCATT | [778] | [‘F’] | [863] | [‘A’] | [‘R’] | 9.26E−27 | 2.38E−25 |
| LENSG00000272405.1 | ENSG00000132692 | BCAN | 5P | Anti | GTTTATG | [2019] | [‘F’] | [1078] | [‘A’] | [‘R’] | 2.60E−25 | 5.63E−24 |
| LENSG00000241764.3 | ENSG00000105879 | CBLL1 | 5P | Same | CCTCTTA | [125] | [‘F’] | [917] | [‘A’] | [‘R’] | 1.86E−24 | 3.82E−23 |
| LENSG00000231105.1 | ENSG00000117298 | ECE1 | 5P | Anti | TGCTTTG | [87] | [‘F’] | [823] | [‘A’] | [‘R’] | 7.71E−23 | 1.38E−21 |
| LENSG00000179988.9 | ENSG00000119965 | C10orf88 | 5P | Anti | TGCTTTG | [132] | [‘F’] | [823] | [‘A’] | [‘R’] | 7.71E−23 | 1.38E−21 |
| LENSG00000231105.1 | ENSG00000117298 | ECE1 | 5P | Same | TGCTTTG | [87] | [‘F’] | [823] | [‘A’] | [‘R’] | 7.71E−23 | 1.38E−21 |
| LENSG00000257913.1 | ENSG00000181929 | PRKAG1 | 5P | Same | TGCTTTG | [226] | [‘F’] | [823] | [‘A’] | [‘R’] | 7.71E−23 | 1.38E−21 |
| LENSG00000264177.1 | ENSG00000214860 | EVPLL | 5P | Same | TGGTTTC | [3739] | [‘F’] | [1141] | [‘A’] | [‘R’] | 2.94E−17 | 3.78E−16 |
| LENSG00000244968.2 | ENSG00000113594 | LIFR | 5P | Anti | CTCTTAA | [178] | [‘R’] | [916] | [‘A’] | [‘R’] | 2.71E−16 | 2.93E−15 |
| LENSG00000203808.6 | ENSG00000112276 | BVES | 5P | Anti | TTAAGTT | [48] | [‘R’] | [1188] | [‘A’] | [‘R’] | 9.76E−16 | 1.00E−14 |
| LENSG00000177917.6 | ENSG00000196504 | PRPF40A | 5P | Anti | TTTGAAC | [206] | [‘R’] | [820] | [‘A’] | [‘R’] | 3.12E−14 | 2.99E−13 |
| LENSG00000177406.4 | ENSG00000171840 | NINJ2 | 5P | Anti | CTTTGCA | [131, | [‘F’, | [1177] | [‘A’] | [‘R’] | 7.96E−14 | 7.44E−13 |
| | | | | | | 199] | ‘R’] | | | | | |
| LENSG00000177406.4 | ENSG00000171840 | NINJ2 | 5P | Same | CTTTGCA | [131, | [‘F’, | [1177] | [‘A’] | [‘R’] | 7.96E−14 | 7.44E−13 |
| | | | | | | 199] | ‘R’] | | | | | |
| LENSG00000267387.1 | ENSG00000130813 | C19orf66 | 5P | Anti | CCTGCTT | [696, | [‘F’, | [825] | [‘A’] | [‘R’] | 1.75E−13 | 1.56E−12 |
| | | | | | | 1227] | ‘F’] | | | | | |
| LENSG00000240207.2 | ENSG00000118849 | PARRES1 | 5P | Same | CCTGCTT | [307] | [‘F’] | [825] | [‘A’] | [‘R’] | 1.75E−13 | 1.56E−12 |
| LENSG00000267387.1 | ENSG00000130813 | C19orf66 | 5P | Same | CCTGCTT | [696, | [‘F’, | [825] | [‘A’] | [‘R’] | 1.75E−13 | 1.56E−12 |
| | | | | | | 1227] | ‘F’] | | | | | |
| LENSG00000161040.12 | ENSG00000128606 | LRRC17 | 5P | Same | CCTGCTT | [302] | [‘F’] | [825] | [‘A’] | [‘R’] | 1.75E−13 | 1.56E−12 |
| LENSG00000186615.6 | ENSG00000126777 | KTN1 | 5P | Anti | TCCCTCT | [280] | [‘F’] | [432, | [‘I’, | [‘R’, | 7.85E−13 | 6.72E−12 |
| | | | | | | | | 919, | ‘A’, | ‘R’, | | |
| | | | | | | | | 1445] | ‘I’] | ‘R’] | | |
| LENSG00000186615.6 | ENSG00000126777 | KTN1 | 5P | Same | TCCCTCT | [280] | [‘F’] | [432, | [‘I’, | [‘R’, | 7.85E−13 | 6.72E−12 |
| | | | | | | | | 919, | ‘A’, | ‘R’, | | |
| | | | | | | | | 1445] | ‘I’] | ‘R’] | | |
| LENSG00000267757.1 | ENSG00000125746 | EML2 | 5P | Same | TCCCTCT | [182] | [‘R’] | [432, | [‘I’, | [‘R’, | 7.85E−13 | 6.72E−12 |
| | | | | | | | | 919, | ‘A’, | ‘R’, | | |
| | | | | | | | | 1445] | ‘I’] | ‘R’] | | |
| LENSG00000255282.2 | ENSG00000196611 | MMP1 | 5P | Anti | TTTGCAA | [15] | [‘R’] | [1176] | [‘A’] | [‘R’] | 1.94E−12 | 1.57E−11 |
| LENSG00000255282.2 | ENSG00000196611 | MMP1 | 5P | Same | TTTGCAA | [15] | [‘R’] | [1176] | [‘A’] | [‘R’] | 1.94E−12 | 1.57E−11 |
| LENSG00000228384.3 | ENSG00000144043 | TEX261 | 5P | Anti | GTTCTTG | [726] | [‘F’] | [1025] | [‘A’] | [‘R’] | 4.69E−11 | 3.44E−10 |
| LENSG00000236295.1 | ENSG00000223874 | AC007557.1 | 5P | Same | TTAATCA | [650] | [‘R’] | [1020, | [‘A’, | [‘F’, | 9.74E−11 | 6.67E−10 |
| | | | | | | | | 913, | ‘A’, | ‘R’, | | |
| | | | | | | | | 1672] | ‘I’] | ‘R’] | | |
| LENSG00000236364.2 | ENSG00000143179 | UCK2 | 5P | Same | CTTTGAA | [647] | [‘F’] | [802, | [‘I’, | [‘F’, | 1.13E−10 | 7.62E−10 |
| | | | | | | | | 821] | ‘A’] | ‘R’] | | |
| LENSG00000258672.1 | ENSG00000036530 | CYP46A1 | 5P | Same | CTTTGAA | [360, | [‘F’, | [802, | [‘I’, | [‘F’, | 1.13E−10 | 7.62E−10 |
| | | | | | | 371] | ‘F’] | 821] | ‘A’] | ‘R’] | | |
| LENSG00000225465.6 | ENSG00000128250 | RFPL1 | 5P | Anti | CTTTGGT | [385] | [‘F’] | [117, | [‘I’, | [‘F’, | 1.47E−10 | 9.75E−10 |
| | | | | | | | | 283, | ‘I’, | ‘F’, | | |
| | | | | | | | | 1144] | ‘A’] | ‘R’] | | |
| LENSG00000259673.1 | ENSG00000189227 | C15orf61 | 5P | Anti | TTCTTGA | [392] | [‘R’] | [1024] | [‘A’] | [‘R’] | 1.75E−10 | 1.11E−09 |
| LENSG00000259673.1 | ENSG00000189227 | C15orf61 | 5P | Same | TTCTTGA | [392] | [‘R’] | [1024] | [‘A’] | [‘R’] | 1.75E−10 | 1.11E−09 |
| LENSG00000272578.1 | ENSG00000159496 | RGL4 | 5P | Anti | TCGTCTT | [201] | [‘R’] | [1048] | [‘A’] | [‘R’] | 3.37E−10 | 2.04E−09 |
| LENSG00000228315.7 | ENSG00000159496 | RGL4 | 5P | Anti | TCGTCTT | [201] | [‘R’] | [1048] | [‘A’] | [‘R’] | 3.37E−10 | 2.04E−09 |
| LENSG00000272578.1 | ENSG00000159496 | RGL4 | 5P | Same | TCGTCTT | [201] | [‘R’] | [1048] | [‘A’] | [‘R’] | 3.37E−10 | 2.04E−09 |
| LENSG00000228315.7 | ENSG00000159496 | RGL4 | 5P | Same | TCGTCTT | [201] | [‘R’] | [1048] | [‘A’] | [‘R’] | 3.37E−10 | 2.04E−09 |
| LENSG00000233175.2 | ENSG00000184922 | FMNL1 | 5P | Anti | ATTTCAC | [423] | [‘F’] | [958] | [‘A’] | [‘R’] | 5.66E−10 | 3.32E−09 |
| LENSG00000273485.1 | ENSG00000138172 | CALHM2 | 5P | Anti | TTCCTAG | [780] | [‘F’] | [855] | [‘A’] | [‘R’] | 5.79E−09 | 3.35E−08 |
| LENSG00000273485.1 | ENSG00000138172 | CALHM2 | 5P | Same | TTCCTAG | [780] | [‘F’] | [855] | [‘A’] | [‘R’] | 5.79E−09 | 3.35E−08 |
| LENSG00000059915.12 | ENSG00000107872 | FBXL15 | 5P | Anti | CACCTCT | [272] | [‘R’] | [954] | [‘A’] | [‘R’] | 2.39E−08 | 1.24E−07 |
| LENSG00000267387.1 | ENSG00000130813 | C19orf66 | 5P | Anti | AATTCCT | [1330] | [‘F’] | [1195, | [‘A’, | [‘R’, | 5.34E−08 | 2.68E−07 |
| | | | | | | | | 1636] | ‘I’] | ‘R’] | | |
| LENSG00000228384.3 | ENSG00000144043 | TEX261 | 5P | Anti | AATTCCT | [1921] | [‘R’] | [1195, | [‘A’, | [‘R’, | 5.34E−08 | 2.68E−07 |
| | | | | | | | | 1636] | ‘I’] | ‘R’] | | |
| LENSG00000267387.1 | ENSG00000130813 | C19orf66 | 5P | Same | AATTCCT | [1330] | [‘F’] | [1195, | [‘A’, | [‘R’, | 5.34E−08 | 2.68E−07 |
| | | | | | | | | 1636] | ‘I’] | ‘R’] | | |
| LENSG00000186615.6 | ENSG00000126777 | KTN1 | 5P | Anti | CCATTAT | [24] | [‘R’] | [861] | [‘A’] | [‘R’] | 8.13E−08 | 4.03E−07 |
| LENSG00000186615.6 | ENSG00000126777 | KTN1 | 5P | Same | CCATTAT | [24] | [‘R’] | [861] | [‘A’] | [‘R’] | 8.13E−08 | 4.03E−07 |
| LENSG00000117543.15 | ENSG00000269175 | AC093157.1 | 5P | Anti | AATGCTT | [778] | [‘F’] | [996] | [‘A’] | [‘R’] | 9.00E−08 | 4.35E−07 |
| LENSG00000273485.1 | ENSG00000138172 | CALHM2 | 5P | Anti | AATGCTT | [396] | [‘R’] | [996] | [‘A’] | [‘R’] | 9.00E−08 | 4.35E−07 |
| LENSG00000272405.1 | ENSG00000132692 | BCAN | 5P | Anti | AATGCTT | [1979] | [‘F’] | [996] | [‘A’] | [‘R’] | 9.00E−08 | 4.35E−07 |
| LENSG00000273485.1 | ENSG00000138172 | CALHM2 | 5P | Same | AATGCTT | [396] | [‘R’] | [996] | [‘A’] | [‘R’] | 9.00E−08 | 4.35E−07 |
| LENSG00000226482.1 | ENSG00000181092 | ADIPOQ | 5P | Same | AATGCTT | [2562] | [‘F’] | [996] | [‘A’] | [‘R’] | 9.00E−08 | 4.35E−07 |
| LENSG00000126226.17 | ENSG00000139842 | CUL4A | 5P | Anti | AGACTTT | [237] | [‘R’] | [1147] | [‘A’] | [‘R’] | 1.04E−07 | 4.98E−07 |
| LENSG00000245482.2 | ENSG00000139133 | ALG10 | 5P | Anti | AGACTTT | [740] | [‘F’] | [1147] | [‘A’] | [‘R’] | 1.04E−07 | 4.98E−07 |
| LENSG00000245482.2 | ENSG00000139133 | ALG10 | 5P | Anti | GAATTTC | [654] | [‘F’] | [960] | [‘A’] | [‘R’] | 2.79E−07 | 1.32E−06 |
| LENSG00000234327.3 | ENSG00000167840 | ZNF232 | 5P | Anti | GAATTTC | [145] | [‘F’] | [960] | [‘A’] | [‘R’] | 2.79E−07 | 1.32E−06 |
| LENSG00000272578.1 | ENSG00000159496 | RGL4 | 5P | Anti | TACTCCC | [189] | [‘R’] | [1166] | [‘A’] | [‘R’] | 1.22E−06 | 5.44E−06 |
| LENSG00000228315.7 | ENSG00000159496 | RGL4 | 5P | Anti | TACTCCC | [189] | [‘R’] | [1166] | [‘A’] | [‘R’] | 1.22E−06 | 5.44E−06 |
| LENSG00000272578.1 | ENSG00000159496 | RGL4 | 5P | Same | TACTCCC | [189] | [‘R’] | [1166] | [‘A’] | [‘R’] | 1.22E−06 | 5.44E−06 |
| LENSG00000228315.7 | ENSG00000159496 | RGL4 | 5P | Same | TACTCCC | [189] | [‘R’] | [1166] | [‘A’] | [‘R’] | 1.22E−06 | 5.44E−06 |
| LENSG00000231105.1 | ENSG00000117298 | ECE1 | 5P | Anti | TCACCTC | [150] | [‘R’] | [955] | [‘A’] | [‘R’] | 3.88E−06 | 1.64E−05 |
| LENSG00000231105.1 | ENSG00000117298 | ECE1 | 5P | Same | TCACCTC | [150] | [‘R’] | [955] | [‘A’] | [‘R’] | 3.88E−06 | 1.64E−05 |
| LENSG00000226937.5 | ENSG00000165512 | ZNF22 | 5P | Anti | AAGTTTC | [431, | [‘R’, | [1186] | [‘A’] | [‘R’] | 6.73E−06 | 2.80E−05 |
| | | | | | | 1407] | ‘R’] | | | | | |
| LENSG00000260852.1 | ENSG00000099364 | FBXL19 | 5P | Anti | CCCTTCC | [131] | [‘R’] | [1205] | [‘A’] | [‘R’] | 6.73E−06 | 2.80E−05 |
| LENSG00000259673.1 | ENSG00000189227 | C15orf61 | 5P | Anti | AAGTTTC | [1136] | [‘F’] | [1186] | [‘A’] | [‘R’] | 6.73E−06 | 2.80E−05 |
| LENSG00000226937.5 | ENSG00000165512 | ZNF22 | 5P | Same | AAGTTTC | [431, | [‘R’, | [1186] | [‘A’] | [‘R’] | 6.73E−06 | 2.80E−05 |
| | | | | | | 1407] | ‘R’] | | | | | |
| LENSG00000259673.1 | ENSG00000189227 | C15orf61 | 5P | Same | AAGTTTC | [1136] | [‘F’] | [1186] | [‘A’] | [‘R’] | 6.73E−06 | 2.80E−05 |
| LENSG00000260852.1 | ENSG00000099364 | FBXL19 | 5P | Same | CCCTTCC | [131] | [‘R’] | [1205] | [‘A’] | [‘R’] | 6.73E−06 | 2.80E−05 |
| LENSG00000267757.1 | ENSG00000125746 | EML2 | 5P | Same | CCCTTCC | [45] | [‘R’] | [1205] | [‘A’] | [‘R’] | 6.73E−06 | 2.80E−05 |
| LENSG00000186615.6 | ENSG00000126777 | KTN1 | 5P | Anti | TCCATTA | [25, | [‘R’, | [862] | [‘A’] | [‘R’] | 1.29E−05 | 5.23E−05 |
| | | | | | | 68, | ‘R’, | | | | | |
| | | | | | | 945] | ‘R’] | | | | | |
| LENSG00000186615.6 | ENSG00000126777 | KTN1 | 5P | Same | TCCATTA | [25, | [‘R’, | [862] | [‘A’] | [‘R’] | 1.29E−05 | 5.23E−05 |
| | | | | | | 68, | ‘R’, | | | | | |
| | | | | | | 945] | ‘R’] | | | | | |
| LENSG00000235703.1 | ENSG00000197021 | CXorf40B | 5P | Anti | ACACTCT | [372] | [‘R’] | [815] | [‘A’] | [‘R’] | 2.04E−05 | 8.04E−05 |
| LENSG00000235703.1 | ENSG00000197021 | CXorf40B | 5P | Same | ACACTCT | [372] | [‘R’] | [815] | [‘A’] | [‘R’] | 2.04E−05 | 8.04E−05 |
| LENSG00000258672.1 | ENSG00000036530 | CYP46A1 | 5P | Same | CACTCCT | [306] | [‘F’] | [1219] | [‘A’] | [‘R’] | 3.83E−05 | 0.000145853 |
| LENSG00000267387.1 | ENSG00000130813 | C19orf66 | 5P | Anti | CTCAGTT | [877, | [‘F’, | [902] | [‘A’] | [‘R’] | 4.11E−05 | 0.000152089 |
| | | | | | | 1253, | ‘F’, | | | | | |
| | | | | | | 942] | ‘R’] | | | | | |
| LENSG00000267387.1 | ENSG00000130813 | C19orf66 | 5P | Same | CTCAGTT | [877, | [‘F’, | [902] | [‘A’] | [‘R’] | 4.11E−05 | 0.000152089 |
| | | | | | | 1253, | ‘F’, | | | | | |
| | | | | | | 942] | ‘R’] | | | | | |
| LENSG00000188735.8 | ENSG00000139725 | RHOF | 5P | Same | CTCAGTT | [59] | [‘R’] | [902] | [‘A’] | [‘R’] | 4.11E−05 | 0.000152089 |
| LENSG00000213904.4 | ENSG00000079435 | LIPE | 5P | Same | CTCAGTT | [17] | [‘R’] | [902] | [‘A’] | [‘R’] | 4.11E−05 | 0.000152089 |
| LENSG00000258672.1 | ENSG00000036530 | CYP46A1 | 5P | Same | CTCAGTT | [272] | [‘F’] | [902] | [‘A’] | [‘R’] | 4.11E−05 | 0.000152089 |
| LENSG00000264177.1 | ENSG00000214860 | EVPLL | 5P | Same | ATTAATG | [3759, | [‘F’, | [1018] | [‘A’] | [‘R’] | 0.000166266 | 0.000560125 |
| | | | | | | 3758] | ‘R’] | | | | | |
| LENSG00000272578.1 | ENSG00000159496 | RGL4 | 5P | Anti | CGTCTTC | [200] | [‘R’] | [1047, | [‘A’, | [‘R’, | 0.000628772 | 0.0020674 |
| | | | | | | | | 1788] | ‘I’] | ‘R’] | | |
| LENSG00000228315.7 | ENSG00000159496 | RGL4 | 5P | Anti | CGTCTTC | [200] | [‘R’] | [1047, | [‘A’, | [‘R’, | 0.000628772 | 0.0020674 |
| | | | | | | | | 1788] | ‘I’] | ‘R’] | | |
| LENSG00000272578.1 | ENSG00000159496 | RGL4 | 5P | Same | CGTCTTC | [200] | [‘R’] | [1047, | [‘A’, | [‘R’, | 0.000628772 | 0.0020674 |
| | | | | | | | | 1788] | ‘I’] | ‘R’] | | |
| LENSG00000228315.7 | ENSG00000159496 | RGL4 | 5P | Same | CGTCTTC | [200] | [‘R’] | [1047, | [‘A’, | [‘R’, | 0.000628772 | 0.0020674 |
| | | | | | | | | 1788] | ‘I’] | ‘R’] | | |
| LENSG00000225988.1 | ENSG00000125869 | LAMP5 | 5P | Same | TCAGCTT | [133] | [‘F’] | [1181] | [‘A’] | [‘R’] | 0.000662996 | 0.00216263 |
| LENSG00000233251.3 | ENSG00000055813 | CCDC85A | 5P | Anti | ATTCCAT | [191] | [‘R’] | [864] | [‘A’] | [‘R’] | 0.00168596 | 0.00528953 |
| LENSG00000245293.2 | ENSG00000155016 | CYP2U1 | 5P | Anti | ACTTTGG | [436] | [‘R’] | [1145] | [‘A’] | [‘R’] | 0.00187758 | 0.00580216 |
| LENSG00000181513.10 | ENSG00000161714 | PLCD3 | 5P | Anti | ACTCCTG | [174] | [‘F’] | [1218] | [‘A’] | [‘R’] | 0.00187583 | 0.00580216 |
| LENSG00000181513.10 | ENSG00000161714 | PLCD3 | 5P | Same | ACTCCTG | [174] | [‘F’] | [1218] | [‘A’] | [‘R’] | 0.00187583 | 0.00580216 |
| LENSG00000245293.2 | ENSG00000155016 | CYP2U1 | 5P | Same | ACTTTGG | [436] | [‘R’] | [1145] | [‘A’] | [‘R’] | 0.00187758 | 0.00580216 |
| LENSG00000225855.2 | ENSG00000160753 | RUSC1 | 5P | Same | ACTCCCC | [28] | [‘R’] | [1165] | [‘A’] | [‘R’] | 0.00214599 | 0.00658209 |
| LENSG00000255808.1 | ENSG00000186642 | PDE2A | 5P | Anti | CTCCCCC | [297, | [‘F’, | [1164] | [‘A’] | [‘R’] | 0.00369813 | 0.0112588 |
| | | | | | | 437] | ‘F’] | | | | | |
| LENSG00000255808.1 | ENSG00000186642 | PDE2A | 5P | Same | CTCCCCC | [297, | [‘F’, | [1164] | [‘A’] | [‘R’] | 0.00369813 | 0.0112588 |
| | | | | | | 437] | ‘F’] | | | | | |
| LENSG00000233175.2 | ENSG00000184922 | FMNL1 | 5P | Anti | CCATACT | [123] | [‘F’] | [1169] | [‘A’] | [‘R’] | 0.00389968 | 0.0117851 |
| LENSG00000225465.6 | ENSG00000128250 | RFPL1 | 5P | Anti | ACTCTAA | [557] | [‘R’] | [813] | [‘A’] | [‘R’] | 0.0105982 | 0.0313372 |
| LENSG00000186615.6 | ENSG00000126777 | KTN1 | 5P | Anti | TCTTCGA | [1027] | [‘F’] | [1045] | [‘A’] | [‘R’] | 0.0108958 | 0.0319188 |
| LENSG00000227375.1 | ENSG00000075711 | DLG1 | 5P | Anti | TCTTCGA | [114] | [‘R’] | [1045] | [‘A’] | [‘R’] | 0.0108958 | 0.0319188 |
| LENSG00000186615.6 | ENSG00000126777 | KTN1 | 5P | Same | TCTTCGA | [1027] | [‘F’] | [1045] | [‘A’] | [‘R’] | 0.0108958 | 0.0319188 |
| LENSG00000186615.6 | ENSG00000126777 | KTN1 | 5P | Anti | GTCTTCG | [1026] | [‘F’] | [1046] | [‘A’] | [‘R’] | 0.0148888 | 0.0424952 |
| LENSG00000186615.6 | ENSG00000126777 | KTN1 | 5P | Same | GTCTTCG | [1026] | [‘F’] | [1046] | [‘A’] | [‘R’] | 0.0148888 | 0.0424952 |
| LENSG00000117543.15 | ENSG00000269175 | AC093157.1 | 5P | Anti | AAATGCT | [777] | [‘F’] | [997] | [‘A’] | [‘R’] | 0.0172869 | 0.0489996 |
| LENSG00000245293.2 | ENSG00000155016 | CYP2U1 | 5P | Anti | AAATGCT | [953] | [‘F’] | [997] | [‘A’] | [‘R’] | 0.0172869 | 0.0489996 |
| LENSG00000273485.1 | ENSG00000138172 | CALHM2 | 5P | Anti | AAATGCT | [397] | [‘R’] | [997] | [‘A’] | [‘R’] | 0.0172869 | 0.0489996 |
| LENSG00000272405.1 | ENSG00000132692 | BCAN | 5P | Anti | AAATGCT | [1978] | [‘F’] | [997] | [‘A’] | [‘R’] | 0.0172869 | 0.0489996 |
| LENSG00000273485.1 | ENSG00000138172 | CALHM2 | 5P | Same | AAATGCT | [397] | [‘R’] | [997] | [‘A’] | [‘R’] | 0.0172869 | 0.0489996 |
| LENSG00000245293.2 | ENSG00000155016 | CYP2U1 | 5P | Same | AAATGCT | [953] | [‘F’] | [997] | [‘A’] | [‘R’] | 0.0172869 | 0.0489996 |
| LENSG00000261519.2 | ENSG00000166548 | TK2 | 5P | Anti | TTGAACA | [43] | [‘F’] | [819] | [‘A’] | [‘R’] | 0.0242818 | 0.06789 |
| LENSG00000141140.12 | ENSG00000184886 | PIGW | 5P | Anti | CCACTCC | [30] | [‘F’] | [490, | [‘I’, | [‘F’, | 0.0331242 | 0.0895662 |
| | | | | | | | | 1220] | ‘A’] | ‘R’] | | |
| LENSG00000226937.5 | ENSG00000165512 | ZNF22 | 5P | Anti | CTCTGGT | [315, | [‘F’, | [986] | [‘A’] | [‘R’] | 0.0346318 | 0.0930306 |
| | | | | | | 1866] | ‘R’] | | | | | |
| LENSG00000226937.5 | ENSG00000165512 | ZNF22 | 5P | Same | CTCTGGT | [315, | [‘F’, | [986] | [‘A’] | [‘R’] | 0.0346318 | 0.0930306 |
| | | | | | | 1866] | ‘R’] | | | | | |
| LENSG00000267387.1 | ENSG00000130813 | C19orf66 | 5P | Anti | CCTCAGT | [876, | [‘F’, | [903] | [‘A’] | [‘R’] | 0.0442964 | 0.11822 |
| | | | | | | 1252, | ‘F’, | | | | | |
| | | | | | | 263, | ‘R’, | | | | | |
| | | | | | | 943] | ‘R’] | | | | | |
| LENSG00000267387.1 | ENSG00000130813 | C19orf66 | 5P | Same | CCTCAGT | [876, | [‘F’, | [903] | [‘A’] | [‘R’] | 0.0442964 | 0.11822 |
| | | | | | | 1252, | ‘F’, | | | | | |
| | | | | | | 263, | ‘R’, | | | | | |
| | | | | | | 943] | ‘R’] | | | | | |
| LENSG00000188735.8 | ENSG00000139725 | RHOF | 5P | Same | CCTCAGT | [60] | [‘R’] | [903] | [‘A’] | [‘R’] | 0.0442964 | 0.11822 |
| LENSG00000258672.1 | ENSG00000036530 | CYP46A1 | 5P | Same | CCTCAGT | [271, | [‘F’, | [903] | [‘A’] | [‘R’] | 0.0442964 | 0.11822 |
| | | | | | | 14] | ‘R’] | | | | | |
| LENSG00000100911.9 | ENSG00000092098 | RNF31 | 5P | Same | GCCCTTC | [291] | [‘R’] | [1206] | [‘A’] | [‘R’] | 0.0458354 | 0.121538 |
| LENSG00000221817.5 | ENSG00000166348 | USP54 | 5P | Anti | AACCTCC | [3410, | [‘F’, | [1039] | [‘A’] | [‘R’] | 0.143866 | 0.355053 |
| | | | | | | 4401, | ‘F’, | | | | | |
| | | | | | | 1240] | ‘R’] | | | | | |
| LENSG00000125967.12 | ENSG00000149609 | C20orf144 | 5P | Same | TCTGGTC | [392] | [‘R’] | [985] | [‘A’] | [‘R’] | 0.143357 | 0.355053 |
| LENSG00000258520.1 | ENSG00000100626 | GALNT16 | 5P | Anti | TAGCTGC | [10] | [‘R’] | [851] | [‘A’] | [‘R’] | 0.156979 | 0.379521 |
| LENSG00000160401.10 | ENSG00000187024 | PTRH1 | 5P | Anti | TGCCCCC | [9] | [‘R’] | [931] | [‘A’] | [‘R’] | 0.315839 | 0.737556 |
| LENSG00000198910.8 | ENSG00000196987 | LCA10 | 5P | Anti | TGCCCCC | [481] | [‘F’] | [931] | [‘A’] | [‘R’] | 0.315839 | 0.737556 |
| LENSG00000160401.10 | ENSG00000187024 | PTRH1 | 5P | Same | TGCCCCC | [9] | [‘R’] | [931] | [‘A’] | [‘R’] | 0.315839 | 0.737556 |
| LENSG00000267163.1 | ENSG00000267680 | ZNF224 | 5P | Anti | CAGTTCC | [405] | [‘R’] | [900] | [‘A’] | [‘R’] | 0.353881 | 0.817107 |
| LENSG00000267163.1 | ENSG00000267680 | ZNF224 | 5P | Same | CAGTTCC | [405] | [‘R’] | [900] | [‘A’] | [‘R’] | 0.353881 | 0.817107 |
| LENSG00000228322.1 | ENSG00000107249 | GLIS3 | 5P | Same | CAGTTCC | [129] | [‘F’] | [900] | [‘A’] | [‘R’] | 0.353881 | 0.817107 |
| LENSG00000261460.1 | ENSG00000213614 | HEXA | 5P | Anti | GGAATAA | [330] | [‘R’] | [858, | [‘A’, | [‘F’, | 0.991811 | 1 |
| | | | | | | | | 1110, | ‘A’, | ‘R’, | | |
| | | | | | | | | 1620] | ‘I’] | ‘R’] | | |
| LENSG00000272476.1 | ENSG00000025796 | SEC63 | 5P | Anti | GGAATAA | [85] | [‘R’] | [858, | [‘A’, | [‘F’, | 0.991811 | 1 |
| | | | | | | | | 1110, | ‘A’, | ‘R’, | | |
| | | | | | | | | 1620] | ‘I’] | ‘R’] | | |
| LENSG00000261460.1 | ENSG00000213614 | HEXA | 5P | Same | GGAATAA | [330] | [‘R’] | [858, | [‘A’, | [‘F’, | 0.991811 | 1 |
| | | | | | | | | 1110, | ‘A’, | ‘R’, | | |
| | | | | | | | | 1620] | ‘I’] | ‘R’] | | |
| LENSG00000245293.2 | ENSG00000155016 | CYP2U1 | 5P | Anti | CAAGAAT | [1299] | [‘R’] | [1006, | [‘A’, | [‘F’, | 0.999998 | 1 |
| | | | | | | | | 963] | ‘A’] | ‘R’] | | |
| LENSG00000245293.2 | ENSG00000155016 | CYP2U1 | 5P | Anti | CCAAGAA | [1194] | [‘F’] | [1005, | [‘A’, | [‘F’, | 1 | 1 |
| | | | | | | | | 964] | ‘A’] | ‘R’] | | |
| LENSG00000245293.2 | ENSG00000155016 | CYP2U1 | 5P | Same | CAAGAAT | [1299] | [‘R’] | [1006, | [‘A’, | [‘F’, | 0.999998 | 1 |
| | | | | | | | | 963] | ‘A’] | ‘R’] | | |
| LENSG00000245293.2 | ENSG00000155016 | CYP2U1 | 5P | Same | CCAAGAA | [1194] | [‘F’] | [1005, | [‘A’, | [‘F’, | 1 | 1 |
| | | | | | | | | 964] | ‘A’] | ‘R’] | | |
| LENSG00000261460.1 | ENSG00000213614 | HEXA | 5P | Anti | GGGAATA | [331] | [‘R’] | [1111, | [‘A’, | [‘R’, | 0.999982 | 1 |
| | | | | | | | | 1621] | ‘I’] | ‘R’] | | |
| LENSG00000273485.1 | ENSG00000138172 | CALHM2 | 5P | Anti | GGCCTCA | [50] | [‘R’] | [905, | [‘A’, | [‘R’, | 0.99998 | 1 |
| | | | | | | | | 1732] | ‘I’] | ‘R’] | | |
| LENSG00000267769.1 | ENSG00000167671 | UBXN6 | 5P | Anti | GGCCTCA | [141] | [‘F’] | [905, | [‘A’, | [‘R’, | 0.99998 | 1 |
| | | | | | | | | 1732] | ‘I’] | ‘R’] | | |
| LENSG00000267387.1 | ENSG00000130813 | C19orf66 | 5P | Anti | GGCCTCA | [874, | [‘F’, | [905, | [‘A’, | [‘R’, | 0.99998 | 1 |
| | | | | | | 1250, | ‘F’, | 1732] | ‘I’] | ‘R’] | | |
| | | | | | | 1051] | ‘R’] | | | | | |
| LENSG00000267387.1 | ENSG00000130813 | C19orf66 | 5P | Same | GGCCTCA | [874, | [‘F’, | [905, | [‘A’, | [‘R’, | 0.99998 | 1 |
| | | | | | | 1250, | ‘F’, | 1732] | ‘I’] | ‘R’] | | |
| | | | | | | 1051] | ‘R’] | | | | | |
| LENSG00000261460.1 | ENSG00000213614 | HEXA | 5P | Same | GGGAATA | [331] | [‘R’] | [1111, | [‘A’, | [‘R’, | 0.999982 | 1 |
| | | | | | | | | 1621] | ‘I’] | ‘R’] | | |
| LENSG00000273485.1 | ENSG00000138172 | CALHM2 | 5P | Same | GGCCTCA | [50] | [‘R’] | [905, | [‘A’, | [‘R’, | 0.99998 | 1 |
| | | | | | | | | 1732] | ‘I’] | ‘R’] | | |
| LENSG00000258672.1 | ENSG00000036530 | CYP46A1 | 5P | Same | GGCCTCA | [269] | [‘F’] | [905, | [‘A’, | [‘R’, | 0.99998 | 1 |
| | | | | | | | | 1732] | ‘I’] | ‘R’] | | |
| LENSG00000258672.1 | ENSG00000036530 | CYP46A1 | 5P | Same | TGGGAAT | [339] | [‘F’] | [1112, | [‘A’, | [‘R’, | 0.875691 | 1 |
| | | | | | | | | 1640] | ‘I’] | ‘R’] | | |
| LENSG00000255119.1 | ENSG00000184224 | C11orf72 | 5P | Same | GCTCCAC | [5] | [‘R’] | [1223, | [‘A’, | [‘R’, | 0.964162 | 1 |
| | | | | | | | | 1348] | ‘I’] | ‘R’] | | |
| LENSG00000125967.12 | ENSG00000149609 | C20orf144 | 5P | Same | GGCCTCA | [319] | [‘F’] | [905, | [‘A’, | [‘R’, | 0.99998 | 1 |
| | | | | | | | | 1732] | ‘I’] | ‘R’] | | |
| LENSG00000261460.1 | ENSG00000213614 | HEXA | 5P | Anti | GGCAAAT | [60] | [‘R’] | [1000] | [‘A’] | [‘R’] | 0.999583 | 1 |
| LENSG00000273151.1 | ENSG00000239857 | GET4 | 5P | Anti | CCCAAAG | [5916, | [‘F’, | [1152] | [‘A’] | [‘R’] | 0.999991 | 1 |
| | | | | | | 1502, | ‘R’, | | | | | |
| | | | | | | 5419] | ‘R’] | | | | | |
| LENSG00000226937.5 | ENSG00000165512 | ZNF22 | 5P | Anti | AACAAAA | [1766] | [‘F’] | [886] | [‘A’] | [‘R’] | 1 | 1 |
| LENSG00000235703.1 | ENSG00000197021 | CXorf40B | 5P | Anti | GCCTCAG | [590] | [‘R’] | [904] | [‘A’] | [‘R’] | 0.999754 | 1 |
| LENSG00000137497.13 | ENSG00000184154 | LRTOMT | 5P | Anti | GAACCCA | [459, | [‘F’, | [1155] | [‘A’] | [‘R’] | 0.999837 | 1 |
| | | | | | | 513] | ‘F’] | | | | | |
| LENSG00000245482.2 | ENSG00000139133 | ALG10 | 5P | Anti | TCCAAGA | [420, | [‘F’, | [965] | [‘A’] | [‘R’] | 0.999992 | 1 |
| | | | | | | 677, | ‘F’, | | | | | |
| | | | | | | 1893] | ‘R’] | | | | | |
| LENSG00000233175.2 | ENSG00000184922 | FMNL1 | 5P | Anti | GGGCCTG | [561, | [‘F’, | [828] | [‘A’] | [‘R’] | 1 | 1 |
| | | | | | | 191] | ‘R’] | | | | | |
| LENSG00000233175.2 | ENSG00000184922 | FMNL1 | 5P | Anti | TGGCAAA | [1] | [‘F’] | [1001] | [‘A’] | [‘R’] | 1 | 1 |
| LENSG00000245293.2 | ENSG00000155016 | CYP2U1 | 5P | Anti | TCCAAGA | [167] | [‘F’] | [965] | [‘A’] | [‘R’] | 0.999992 | 1 |
| LENSG00000247708.3 | ENSG00000168818 | STX18 | 5P | Anti | CTAGCTG | [217] | [‘F’] | [852] | [‘A’] | [‘R’] | 0.986328 | 1 |
| LENSG00000273485.1 | ENSG00000138172 | CALHM2 | 5P | Anti | GCCTCAG | [49] | [‘R’] | [904] | [‘A’] | [‘R’] | 0.999754 | 1 |
| LENSG00000273485.1 | ENSG00000138172 | CALHM2 | 5P | Anti | CAAATGC | [398] | [‘R’] | [998] | [‘A’] | [‘R’] | 0.971655 | 1 |
| LENSG00000168569.7 | ENSG00000162227 | TAF6L | 5P | Anti | AAGCTGC | [222] | [‘R’] | [1130] | [‘A’] | [‘R’] | 0.999922 | 1 |
| LENSG00000267769.1 | ENSG00000167671 | UBXN6 | 5P | Anti | CGGCTCG | [306, | [‘F’, | [834] | [‘A’] | [‘R’] | 1 | 1 |
| | | | | | | 68] | ‘R’] | | | | | |
| LENSG00000132581.5 | ENSG00000109111 | SUPT6H | 5P | Anti | GAAAACA | [138] | [‘R’] | [1012] | [‘A’] | [‘R’] | 1 | 1 |
| LENSG00000267387.1 | ENSG00000130813 | C19orf66 | 5P | Anti | AGCTGCC | [1220] | [‘F’] | [1129] | [‘A’] | [‘R’] | 0.999972 | 1 |
| LENSG00000267387.1 | ENSG00000130813 | C19orf66 | 5P | Anti | GCCTCAG | [875, | [‘F’, | [904] | [‘A’] | [‘R’] | 0.999754 | 1 |
| | | | | | | 1251, | ‘F’, | | | | | |
| | | | | | | 944] | ‘R’] | | | | | |
| LENSG00000267387.1 | ENSG00000130813 | C19orf66 | 5P | Anti | TGGCCTC | [873, | [‘F’, | [906] | [‘A’] | [‘R’] | 0.804552 | 1 |
| | | | | | | 1052] | ‘R’] | | | | | |
| LENSG00000267387.1 | ENSG00000130813 | C19orf66 | 5P | Anti | GCTGCCC | [1221] | [‘F’] | [1128] | [‘A’] | [‘R’] | 1 | 1 |
| LENSG00000267387.1 | ENSG00000130813 | C19orf66 | 5P | Anti | CTCCTGG | [1298] | [‘F’] | [1217] | [‘A’] | [‘R’] | 0.983765 | 1 |
| LENSG00000248724.2 | ENSG00000113971 | NPHP3 | 5P | Anti | ATGAAAA | [1799] | [‘F’] | [1014] | [‘A’] | [‘R’] | 0.999366 | 1 |
| LENSG00000245330.4 | ENSG00000155792 | DEPTOR | 5P | Anti | AAAATAG | [221] | [‘R’] | [883] | [‘A’] | [‘R’] | 0.99597 | 1 |
| LENSG00000186615.6 | ENSG00000126777 | KTN1 | 5P | Anti | CAAAGAC | [1082] | [‘F’] | [1150] | [‘A’] | [‘R’] | 1 | 1 |
| LENSG00000186615.6 | ENSG00000126777 | KTN1 | 5P | Anti | CTTCGAA | [1028] | [‘F’] | [1044] | [‘A’] | [‘R’] | 0.478428 | 1 |
| LENSG00000198910.8 | ENSG00000196987 | LCA10 | 5P | Anti | GGTGCCC | [468] | [‘F’] | [1209] | [‘A’] | [‘R’] | 0.999997 | 1 |
| LENSG00000198910.8 | ENSG00000196987 | LCA10 | 5P | Anti | GGGCCTG | [436] | [‘F’] | [828] | [‘A’] | [‘R’] | 1 | 1 |
| LENSG00000196220.11 | ENSG00000134077 | THUMPD3 | 5P | Anti | TCCAAGA | [315] | [‘R’] | [965] | [‘A’] | [‘R’] | 0.999992 | 1 |
| LENSG00000255808.1 | ENSG00000186642 | PDE2A | 5P | Anti | GCCGCAT | [704] | [‘R’] | [1099] | [‘A’] | [‘R’] | 0.967908 | 1 |
| LENSG00000272405.1 | ENSG00000132692 | BCAN | 5P | Anti | GGAACCC | [2047] | [‘F’] | [1156] | [‘A’] | [‘R’] | 1 | 1 |
| LENSG00000178803.6 | ENSG00000128271 | ADORA2A | 5P | Anti | GGAACCC | [210] | [‘F’] | [1156] | [‘A’] | [‘R’] | 1 | 1 |
| LENSG00000251405.2 | ENSG00000172548 | NIPAL4 | 5P | Anti | CAAAGAC | [9] | [‘R’] | [1150] | [‘A’] | [‘R’] | 1 | 1 |
| LENSG00000137133.6 | ENSG00000137103 | TMEM8B | 5P | Anti | GAAGCTG | [984] | [‘R’] | [1131] | [‘A’] | [‘R’] | 1 | 1 |
| LENSG00000258584.1 | ENSG00000140067 | FAM181A | 5P | Anti | GCTCTGG | [53, | [‘R’, | [987] | [‘A’] | [‘R’] | 0.999869 | 1 |
| | | | | | | 482] | ‘R’] | | | | | |
| LENSG00000227218.3 | ENSG00000136908 | DPM2 | 5P | Anti | ATGGGAA | [739] | [‘F’] | [1113] | [‘A’] | [‘R’] | 1 | 1 |
| LENSG00000178803.6 | ENSG00000100024 | UPB1 | 5P | Anti | CCCAAAG | [152, | [‘F’, | [1152] | [‘A’] | [‘R’] | 0.999991 | 1 |
| | | | | | | 432] | ‘F’] | | | | | |
| LENSG00000205913.2 | ENSG00000167978 | SRKM2 | 5P | Anti | GGGCCTG | [141] | [‘R’] | [828] | [‘A’] | [‘R’] | 1 | 1 |
| LENSG00000225988.1 | ENSG00000125869 | LAMP5 | 5P | Same | ACAAAAT | [394, | [‘R’, | [885] | [‘A’] | [‘R’] | 0.99847 | 1 |
| | | | | | | 404, | ‘R’, | | | | | |
| | | | | | | 419] | ‘R’] | | | | | |
| LENSG00000267387.1 | ENSG00000130813 | C19orf66 | 5P | Same | AGCTGCC | [1220] | [‘F’] | [1129] | [‘A’] | [‘R’] | 0.999972 | 1 |
| LENSG00000267387.1 | ENSG00000130813 | C19orf66 | 5P | Same | GCCTCAG | [875, | [‘F’, | [904] | [‘A’] | [‘R’] | 0.999754 | 1 |
| | | | | | | 1251, | ‘F’, | | | | | |
| | | | | | | 944] | ‘R’] | | | | | |
| LENSG00000267387.1 | ENSG00000130813 | C19orf66 | 5P | Same | TGGCCTC | [873, | [‘F’, | [906] | [‘A’] | [‘R’] | 0.804552 | 1 |
| | | | | | | 1052] | ‘R’] | | | | | |
| LENSG00000267387.1 | ENSG00000130813 | C19orf66 | 5P | Same | GCTGCCC | [1221] | [‘F’] | [1128] | [‘A’] | [‘R’] | 1 | 1 |
| LENSG00000267387.1 | ENSG00000130813 | C19orf66 | 5P | Same | CTCCTGG | [1298] | [‘F’] | [1217] | [‘A’] | [‘R’] | 0.983765 | 1 |
| LENSG00000188735.8 | ENSG00000139725 | RHOF | 5P | Same | GCCTCAG | [61] | [‘R’] | [904] | [‘A’] | [‘R’] | 0.999754 | 1 |
| LENSG00000262999.1 | ENSG00000188897 | CTD- | 5P | Same | GAATGCC | [208] | [‘R’] | [934] | [‘A’] | [‘R’] | 0.999931 | 1 |
| | | 3088G3.8 | | | | | | | | | | |
| LENSG00000235703.1 | ENSG00000197021 | CXorf40B | 5P | Same | GCCTCAG | [590] | [‘R’] | [904] | [‘A’] | [‘R’] | 0.999754 | 1 |
| LENSG00000258334.1 | ENSG00000135406 | PRPH | 5P | Same | AGCTGCC | [10] | [‘R’] | [1129] | [‘A’] | [‘R’] | 0.999972 | 1 |
| LENSG00000258334.1 | ENSG00000135406 | PRPH | 5P | Same | GGAAGCT | [13] | [‘R’] | [1132] | [‘A’] | [‘R’] | 1 | 1 |
| LENSG00000258334.1 | ENSG00000135406 | PRPH | 5P | Same | GAAGCTG | [12] | [‘R’] | [1131] | [‘A’] | [‘R’] | 1 | 1 |
| LENSG00000264177.1 | ENSG00000214860 | EVPLL | 5P | Same | CCTGGTG | [211, | [‘F’, | [1215] | [‘A’] | [‘R’] | 0.999063 | 1 |
| | | | | | | 1080, | ‘R’, | | | | | |
| | | | | | | 2618] | ‘R’] | | | | | |
| LENSG00000261460.1 | ENSG00000213614 | HEXA | 5P | Same | GGCAAAT | [60] | [‘R’] | [1000] | [‘A’] | [‘R’] | 0.999583 | 1 |
| LENSG00000178803.6 | ENSG00000128271 | ADOPA2A | 5P | Same | GGAACCC | [210] | [‘F’] | [1156] | [‘A’] | [‘R’] | 1 | 1 |
| LENSG00000260625.1 | ENSG00000140688 | C16orf58 | 5P | Same | GCCTCAG | [374] | [‘F’] | [904] | [‘A’] | [‘R’] | 0.999754 | 1 |
| LENSG00000178803.6 | ENSG00000100024 | UPB1 | 5P | Same | CCCAAAG | [152, | [‘F’, | [1152] | [‘A’] | [‘R’] | 0.999991 | 1 |
| | | | | | | 432] | ‘F’] | | | | | |
| LENSG00000273485.1 | ENSG00000138172 | CALHM2 | 5P | Same | GCCTCAG | [49] | [‘R’] | [904] | [‘A’] | [‘R’] | 0.999754 | 1 |
| LENSG00000273485.1 | ENSG00000138172 | CALHM2 | 5P | Same | CAAATGC | [398] | [‘R’] | [998] | [‘A’] | [‘R’] | 0.971655 | 1 |
| LENSG00000258672.1 | ENSG00000036530 | CYP46A1 | 5P | Same | AACACTC | [304] | [‘F’] | [816] | [‘A’] | [‘R’] | 0.558887 | 1 |
| LENSG00000258672.1 | ENSG00000036530 | CYP46A1 | 5P | Same | GCCTCAG | [270] | [‘F’] | [904] | [‘A’] | [‘R’] | 0.999754 | 1 |
| LENSG00000168569.7 | ENSG00000162227 | TAF6L | 5P | Same | AAGCTGC | [222] | [‘R’] | [1130] | [‘A’] | [‘R’] | 0.999922 | 1 |
| LENSG00000196220.11 | ENSG00000134077 | THUMPD3 | 5P | Same | TCCAAGA | [315] | [‘R’] | [965] | [‘A’] | [‘R’] | 0.999992 | 1 |
| LENSG00000226937.5 | ENSG00000165512 | ZNF22 | 5P | Same | AACAAAA | [1766] | [‘F’] | [886] | [‘A’] | [‘R’] | 1 | 1 |
| LENSG00000226482.1 | ENSG00000181092 | ADIPOQ | 5P | Same | ACAAAAT | [2619] | [‘F’] | [885] | [‘A’] | [‘R’] | 0.99847 | 1 |
| LENSG00000226482.1 | ENSG00000181092 | ADIPOQ | 5P | Same | GCTCTGG | [2463] | [‘F’] | [987] | [‘A’] | [‘R’] | 0.999869 | 1 |
| LENSG00000226482.1 | ENSG00000181092 | ADIPOQ | 5P | Same | CAAAATA | [1801, | [‘F’, | [884] | [‘A’] | [‘R’] | 0.947478 | 1 |
| | | | | | | 2620] | ‘F’] | | | | | |
| LENSG00000226482.1 | ENSG00000181092 | ADIPOQ | 5P | Same | GCCTCAG | [2532, | [‘F’, | [904] | [‘A’] | [‘R’] | 0.999754 | 1 |
| | | | | | | 1283, | ‘R’, | | | | | |
| | | | | | | 2002] | ‘R’] | | | | | |
| LENSG00000226482.1 | ENSG00000181092 | ADIPOQ | 5P | Same | AAAATAG | [2621] | [‘F’] | [883] | [‘A’] | [‘R’] | 0.99597 | 1 |
| LENSG00000255119.1 | ENSG00000184224 | C11orf72 | 5P | Same | ACAAAAT | [246] | [‘R’] | [885] | [‘A’] | [‘R’] | 0.99847 | 1 |
| LENSG00000254602.1 | ENSG00000166793 | YPEL4 | 5P | Same | CTTGGCA | [697, | [‘F’, | [1003] | [‘A’] | [‘R’] | 0.495606 | 1 |
| | | | | | | 144] | ‘R’] | | | | | |
| LENSG00000258584.1 | ENSG00000140067 | FAM181A | 5P | Same | GCTCTGG | [53, | [‘R’, | [987] | [‘A’] | [‘R’] | 0.999869 | 1 |
| | | | | | | 482] | ‘R’] | | | | | |
| LENSG00000137497.13 | ENSG00000184154 | LRTOMT | 5P | Same | GAACCCA | [459, | [‘F’, | [1155] | [‘A’] | [‘R’] | 0.999837 | 1 |
| | | | | | | 513] | ‘F’] | | | | | |
| LENSG00000161040.12 | ENSG00000128606 | LRRC17 | 5P | Same | AAAATAG | [383] | [‘F’] | [883] | [‘A’] | [‘R’] | 0.99597 | 1 |
| LENSG00000266962.1 | ENSG00000108786 | HSD17B1 | 5P | Same | ACCAACA | [665, | [‘F’, | [889] | [‘A’] | [‘R’] | 0.999995 | 1 |
| | | | | | | 1156, | ‘F’, | | | | | |
| | | | | | | 1395] | ‘R’] | | | | | |
| LENSG00000266962.1 | ENSG00000108786 | HSD17B1 | 5P | Same | TCTAGCG | [587] | [‘F’] | [950] | [‘A’] | [‘R’] | 0.544959 | 1 |
| LENSG00000266962.1 | ENSG00000108786 | HSD17B1 | 5P | Same | CCTGGTG | [568] | [‘F’] | [1215] | [‘A’] | [‘R’] | 0.999063 | 1 |
| LENSG00000266962.1 | ENSG00000108786 | HSD17B1 | 5P | Same | CTAGCGG | [588] | [‘F’] | [949] | [‘A’] | [‘R’] | 0.999999 | 1 |
| LENSG00000257985.1 | ENSG00000257955 | RP1- | 5P | Same | GCAAATG | [435] | [‘F’] | [999] | [‘A’] | [‘R’] | 0.976881 | 1 |
| | | 228P16.5 | | | | | | | | | | |
| LENSG00000257985.1 | ENSG00000257955 | RP1- | 5P | Same | CCCAAAG | [204] | [‘R’] | [1152] | [‘A’] | [‘R’] | 0.999991 | 1 |
| | | 228P16.5 | | | | | | | | | | |
| LENSG00000125967.12 | ENSG00000149609 | C20orf144 | 5P | Same | GCCTCAG | [320] | [‘F’] | [904] | [‘A’] | [‘R’] | 0.999754 | 1 |
| LENSG00000125967.12 | ENSG00000149609 | C20orf144 | 5P | Same | CTGGTCC | [654] | [‘R’] | [984] | [‘A’] | [‘R’] | 0.993655 | 1 |
| LENSG00000186615.6 | ENSG00000126777 | KTN1 | 5P | Same | CAAAGAC | [1082] | [‘F’] | [1150] | [‘A’] | [‘R’] | 1 | 1 |
| LENSG00000186615.6 | ENSG00000126777 | KTN1 | 5P | Same | CTTCGAA | [1028] | [‘F’] | [1044] | [‘A’] | [‘R’] | 0.478428 | 1 |
| LENSG00000247708.3 | ENSG00000168818 | STX18 | 5P | Same | CTAGCTG | [217] | [‘F’] | [852] | [‘A’] | [‘R’] | 0.986328 | 1 |
| LENSG00000245293.2 | ENSG00000155016 | CYP2U1 | 5P | Same | TCCAAGA | [167] | [‘F’] | [965] | [‘A’] | [‘R’] | 0.999992 | 1 |
| LENSG00000251405.2 | ENSG00000172548 | NIPAL4 | 5P | Same | CAAAGAC | [9] | [‘R’] | [1150] | [‘A’] | [‘R’] | 1 | 1 |
| LENSG00000255808.1 | ENSG00000186642 | PDE2A | 5P | Same | GCCGCAT | [704] | [‘R’] | [1099] | [‘A’] | [‘R’] | 0.967908 | 1 |
| LENSG00000255389.1 | ENSG00000056972 | TPAF3IP2 | 5P | Anti | CAGGCTC | [486] | [‘R’] | [437, | [‘I’, | [‘R’, | 0.78438 | 1 |
| | | | | | | | | 1226] | ‘A’] | ‘R’] | | |
| LENSG00000059915.12 | ENSG00000107872 | FBXL15 | 5P | Anti | AAAACCA | [150] | [‘F’] | [228, | [‘I’, | [‘F’, | 1 | 1 |
| | | | | | | | | 892] | ‘A’] | ‘R’] | | |
| LENSG00000267387.1 | ENSG00000130813 | C19orf66 | 5P | Anti | CAGGCTC | [1184] | [‘F’] | [437, | [‘I’, | [‘R’, | 0.78438 | 1 |
| | | | | | | | | 1226] | ‘A’] | ‘R’] | | |
| LENSG00000272578.1 | ENSG00000159496 | RGL4 | 5P | Anti | AGGCTCC | [162] | [‘R’] | [436, | [‘I’, | [‘R’, | 0.999943 | 1 |
| | | | | | | | | 1225] | ‘A’] | ‘R’] | | |
| LENSG00000234775.1 | ENSG00000163485 | ADOPA1 | 5P | Anti | CAGGCTC | [225] | [‘R’] | [437, | [‘I’, | [‘R’, | 0.78438 | 1 |
| | | | | | | | | 1226] | ‘A’] | ‘R’] | | |
| LENSG00000228384.3 | ENSG00000144043 | TEX261 | 5P | Anti | GGGTCAT | [2987] | [‘F’] | [1655, | [‘I’, | [‘F’, | 0.504667 | 1 |
| | | | | | | | | 1118] | ‘A’] | ‘R’] | | |
| LENSG00000186615.6 | ENSG00000126777 | KTN1 | 5P | Anti | CAGGCTC | [1055] | [‘F’] | [437, | [‘I’, | [‘R’, | 0.78438 | 1 |
| | | | | | | | | 1226] | ‘A’] | ‘R’] | | |
| LENSG00000228315.7 | ENSG00000159496 | RGL4 | 5P | Anti | AGGCTCC | [162] | [‘R’] | [436, | [‘I’, | [‘R’, | 0.999943 | 1 |
| | | | | | | | | 1225] | ‘A’] | ‘R’] | | |
| LENSG00000255389.1 | ENSG00000056972 | TPAF3IP2 | 5P | Same | CAGGCTC | [486] | [‘R’] | [437, | [‘I’, | [‘R’, | 0.78438 | 1 |
| | | | | | | | | 1226] | ‘A’] | ‘R’] | | |
| LENSG00000267387.1 | ENSG00000130813 | C19orf66 | 5P | Same | CAGGCTC | [1184] | [‘F’] | [437, | [‘I’, | [‘R’, | 0.78438 | 1 |
| | | | | | | | | 1226] | ‘A’] | ‘R’] | | |
| LENSG00000271122.1 | ENSG00000122557 | HERPUD2 | 5P | Same | GTGGTGC | [1633] | [‘F’] | [1324, | [‘I’, | [‘F’, | 0.995808 | 1 |
| | | | | | | | | 1211] | ‘A’] | ‘R’] | | |
| LENSG00000272578.1 | ENSG00000159496 | RGL4 | 5P | Same | AGGCTCC | [162] | [‘R’] | [436, | [‘I’, | [‘R’, | 0.999943 | 1 |
| | | | | | | | | 1225] | ‘A’] | ‘R’] | | |
| LENSG00000161040.12 | ENSG00000128606 | LRRC17 | 5P | Same | CATGGCC | [413] | [‘F’] | [1330, | [‘I’, | [‘F’, | 0.998686 | 1 |
| | | | | | | | | 908] | ‘A’] | ‘R’] | | |
| LENSG00000161040.12 | ENSG00000128606 | LRRC17 | 5P | Same | GGGTCAT | [323] | [‘F’] | [1655, | [‘I’, | [‘F’, | 0.504667 | 1 |
| | | | | | | | | 1118] | ‘A’] | ‘R’] | | |
| LENSG00000228315.7 | ENSG00000159496 | RGL4 | 5P | Same | AGGCTCC | [162] | [‘R’] | [436, | [‘I’, | [‘R’, | 0.999943 | 1 |
| | | | | | | | | 1225] | ‘A’] | ‘R’] | | |
| LENSG00000186615.6 | ENSG00000126777 | KTN1 | 5P | Same | CAGGCTC | [1055] | [‘F’] | [437, | [‘I’, | [‘R’, | 0.78438 | 1 |
| | | | | | | | | 1226] | ‘A’] | ‘R’] | | |
| LENSG00000234028.3 | ENSG00000172071 | EIF2AK3 | 5P | Anti | CCCGGCC | [140] | [‘R’] | [257, | [‘I’, | [‘F’, | 0.999957 | 1 |
| | | | | | | | | 1265, | ‘I’, | ‘F’, | | |
| | | | | | | | | 260, | ‘I’, | ‘R’, | | |
| | | | | | | | | 927] | ‘A’] | ‘R’] | | |
| LENSG00000115840.9 | ENSG00000172878 | METAP1D | 5P | Anti | ACCAAAG | [235] | [‘F’] | [1144, | [‘A’, | [‘F’, | NA | NA |
| | | | | | | | | 117, | ‘I’, | ‘R’, | | |
| | | | | | | | | 283] | ‘I’] | ‘R’] | | |
| LENSG00000115840.9 | ENSG00000172878 | METAP1D | 5P | Anti | GGAAACC | [925] | [‘R’] | [1140, | [‘A’, | [‘F’, | NA | NA |
| | | | | | | | | 1255, | ‘I’, | ‘F’, | | |
| | | | | | | | | 1835] | ‘I’] | ‘R’] | | |
| LENSG00000255542.1 | ENSG00000110436 | SLC1A2 | 5P | Same | CACCACC | [228] | [‘F’] | [1212, | [‘A’, | [‘F’, | NA | NA |
| | | | | | | | | 1320, | ‘I’, | ‘R’, | | |
| | | | | | | | | 1323] | ‘I’] | ‘R’] | | |
| LENSG00000226937.5 | ENSG00000165512 | ZNF22 | 5P | Anti | AGGTGAA | [1080, | [‘F’, | [956, | [‘A’, | [‘F’, | NA | NA |
| | | | | | | 2401] | ‘F’] | 1844] | ‘I’] | ‘F’] | | |
| LENSG00000248636.2 | ENSG00000111725 | PRKAB1 | 5P | Anti | AAAGGAA | [161] | [‘F’] | [1193, | [‘A’, | [‘F’, | NA | NA |
| | | | | | | | | 114] | ‘I’] | R’] | | |
| LENSG00000130821.11 | ENSG00000130822 | PNCK | 5P | Anti | GTGGAGC | [373] | [‘F’] | [1223, | [‘A’, | [‘F’, | NA | NA |
| | | | | | | | | 1348] | ‘I’] | ‘F’] | | |
| LENSG00000236432.3 | ENSG00000168958 | MFF | 5P | Anti | TAAAGGA | [205] | [‘F’] | [1192, | [‘A’, | [‘F’, | NA | NA |
| | | | | | | | | 576] | ‘I’] | ‘R’] | | |
| LENSG00000259673.1 | ENSG00000189227 | C15orf61 | 5P | Anti | TTCAAAG | [287] | [‘F’] | [821, | [‘A’, | [‘F’, | NA | NA |
| | | | | | | | | 802] | ‘I’] | ‘R’] | | |
| LENSG00000236404.4 | ENSG00000147852 | VLDLR | 5P | Anti | CTAGAGG | [295] | [‘F’] | [952, | [‘A’, | [‘F’, | NA | NA |
| | | | | | | | | 1810] | ‘I’] | ‘F’] | | |
| LENSG00000246090.2 | ENSG00000187758 | ADH1A | 5P | Anti | TAATCAA | [1193] | [‘F’] | [1021, | [‘A’, | [‘F’, | NA | NA |
| | | | | | | | | 1671] | ‘I’] | ‘R’] | | |
| LENSG00000267387.1 | ENSG00000130813 | C19orf66 | 5P | Anti | GCACCAC | [843] | [‘F’] | [1211, | [‘A’, | [‘F’, | NA | NA |
| | | | | | | | | 1324] | ‘I’] | ‘R’] | | |
| LENSG00000267387.1 | ENSG00000130813 | C19orf66 | 5P | Anti | GGGAAAC | [938] | [‘F’] | [1139, | [‘A’, | [‘F’, | NA | NA |
| | | | | | | | | 1254] | ‘I’] | ‘F’] | | |
| LENSG00000228384.3 | ENSG00000144043 | TEX261 | 5P | Anti | AAAGGAA | [628, | [‘R’, | [1193, | [‘A’, | [‘F’, | NA | NA |
| | | | | | | 1804] | ‘R’] | 114] | ‘I’] | ‘R’] | | |
| LENSG00000186615.6 | ENSG00000126777 | KTN1 | 5P | Anti | ATTCCCA | [1135] | [‘F’] | [1112, | [‘A’, | [‘F’, | NA | NA |
| | | | | | | | | 1640] | ‘I’] | ‘F’] | | |
| LENSG00000206417.4 | ENSG00000184897 | H1FX | 5P | Anti | GGGAAAC | [1055] | [‘F’] | [1139, | [‘A’, | [‘F’, | NA | NA |
| | | | | | | | | 1254] | ‘I’] | ‘F’] | | |
| LENSG00000183242.7 | ENSG00000184937 | WT1 | 5P | Anti | TGAGGCC | [44] | [‘F’] | [905, | [‘A’, | [‘F’, | NA | NA |
| | | | | | | | | 1732] | ‘I’] | ‘F’] | | |
| LENSG00000268677.1 | ENSG00000224420 | ADM5 | 5P | Anti | GCCCGAG | [377] | [‘F’] | [831, | [‘A’, | [‘F’, | NA | NA |
| | | | | | | | | 303] | ‘I’] | ‘R’] | | |
| LENSG00000178803.6 | ENSG00000100024 | UPB1 | 5P | Anti | TGAGGCC | [1016] | [‘F’] | [905, | [‘A’, | R | NA | NA |
| | | | | | | | | 1732] | ‘I’] | | | |
| LENSG00000183242.7 | ENSG00000184937 | WT1 | 5P | Same | TGAGGCC | [44] | [‘F’] | [905, | [‘A’, | [‘F’, | NA | NA |
| | | | | | | | | 1732] | ‘I’] | ‘F’] | | |
| LENSG00000236404.4 | ENSG00000147852 | VLDLR | 5P | Same | CTAGAGG | [295] | [‘F’] | [952, | [‘A’, | [‘F’, | NA | NA |
| | | | | | | | | 1810] | ‘I’] | ‘F’] | | |
| LENSG00000267387.1 | ENSG00000130813 | C19orf66 | 5P | Same | GCACCAC | [843] | [‘F’] | [1211, | [‘A’, | [‘F’, | NA | NA |
| | | | | | | | | 1324] | ‘I’] | ‘R’] | | |
| LENSG00000267387.1 | ENSG00000130813 | C19orf66 | 5P | Same | GGGAAAC | [938] | [‘F’] | [1139, | [‘A’, | [‘F’, | NA | NA |
| | | | | | | | | 1254] | ‘I’] | ‘F’] | | |
| LENSG00000236432.3 | ENSG00000168958 | MFF | 5P | Same | TAAAGGA | [205] | [‘F] | [1192 | [‘A’, | [‘F’, | NA | NA |
| | | | | | | | | 576] | ‘I’] | ‘R’] | | |
| LENSG00000178803.6 | ENSG00000100024 | UPB1 | 5P | Same | TGAGGCC | [1016] | [‘F’] | [905, | [‘A’, | R | NA | NA |
| | | | | | | | | 1732] | ‘I’] | | | |
| LENSG00000226937.5 | ENSG00000165512 | ZNF22 | 5P | Same | AGGTGAA | [1080, | [‘F’, | [956, | [‘A’, | [‘F’, | NA | NA |
| | | | | | | 2401] | ‘F’] | 1844] | ‘I’] | ‘F’] | | |
| LENSG00000259673.1 | ENSG00000189227 | C15orf61 | 5P | Same | TTCAAAG | [287] | [‘F’] | [821, | [‘A’, | [‘F’, | NA | NA |
| | | | | | | | | 802] | ‘I’] | ‘R’] | | |
| LENSG00000246090.2 | ENSG00000187758 | ADH1A | 5P | Same | TAATCAA | [1193] | [‘F’] | [1021, | [‘A’, | [‘F’, | NA | NA |
| | | | | | | | | 1671] | ‘I’] | ‘R’] | | |
| LENSG00000186615.6 | ENSG00000126777 | KTN1 | 5P | Same | ATTCCCA | [1135] | [‘F’] | [1112, | [‘A’, | [‘F’, | NA | NA |
| | | | | | | | | 1640] | ‘I’] | ‘F’] | | |
| LENSG00000250309.2 | ENSG00000164591 | MYOZ3 | 5P | Same | CCCATGA | [141] | [‘F’] | [1115, | [‘A’, | [‘F’, | NA | NA |
| | | | | | | | | 1626] | ‘I’] | ‘F’] | | |
| LENSG00000261570.1 | ENSG00000006530 | AGK | 5P | Anti | GAGGCCA | [127] | [‘F’] | [906] | [‘A’] | [‘F’] | NA | NA |
| LENSG00000255389.1 | ENSG00000056972 | TRAF3IP2 | 5P | Anti | AAGCAGG | [83, | [‘R’, | [825] | [‘A’] | [‘F’] | NA | NA |
| | | | | | | 477] | ‘R’] | | | | | |
| LENSG00000255389.1 | ENSG00000056972 | TRAF3IP2 | 5P | Anti | ATGATTA | [192] | [‘F’] | [912] | [‘A’] | R | NA | NA |
| LENSG00000266304.1 | ENSG00000266258 | RP11- | 5P | Anti | AACTTAA | [305] | [‘F’] | [1188] | [‘A’] | [‘F’] | NA | NA |
| | | 41O4.1 | | | | | | | | | | |
| LENSG00000266304.1 | ENSG00000266258 | RP11- | 5P | Anti | GAGGCCA | [292] | [‘F’] | [906] | [‘A’] | [‘F’] | NA | NA |
| | | 41O4.1 | | | | | | | | | | |
| LENSG00000273151.1 | ENSG00000239857 | GET4 | 5P | Anti | TAAGAGG | [5898, | [‘F’, | [917] | [‘A’] | R | NA | NA |
| | | | | | | 3454] | ‘R’] | | | | | |
| LENSG00000273151.1 | ENSG00000239857 | GET4 | 5P | Anti | AGAGGTG | [2427, | [‘F’, | [954] | [‘A’] | [‘F’] | NA | NA |
| | | | | | | 5900] | ‘F’] | | | | | |
| LENSG00000273151.1 | ENSG00000239857 | GET4 | 5P | Anti | GAGGTTC | [5870] | [‘F’] | [1040] | [‘A’] | [‘F’] | NA | NA |
| LENSG00000264589.1 | ENSG00000186868 | MAPT | 5P | Anti | CTGAGGC | [134] | [‘R’] | [904] | [‘A’] | [‘F’] | NA | NA |
| LENSG00000169442.4 | ENSG00000158062 | UBXN11 | 5P | Anti | TGCCAAG | [389] | [‘F’] | [1003] | [‘A’] | [‘F’] | NA | NA |
| LENSG00000169442.4 | ENSG00000158062 | UBXN11 | 5P | Anti | CTAGGAA | [89] | [‘F’] | [855] | [‘A’] | [‘F’] | NA | NA |
| LENSG00000059915.12 | ENSG00000107872 | FBXL15 | 5P | Anti | GGGCACC | [275] | [‘R’] | [1209] | [‘A’] | R | NA | NA |
| LENSG00000229619.3 | ENSG00000152601 | MBNL1 | 5P | Anti | AATGGAA | [32] | [‘R’] | [863] | [‘A’] | R | NA | NA |
| LENSG00000229619.3 | ENSG00000152601 | MBNL1 | 5P | Anti | TTTTCAT | [563, | [‘F’, | [1014] | [‘A’] | R | NA | NA |
| | | | | | | 815] | ‘F’] | | | | | |
| LENSG00000226937.5 | ENSG00000165512 | ZNF22 | 5P | Anti | AGTGTTC | [2433, | [‘F’, | [817] | [‘A’] | R | NA | NA |
| | | | | | | 2109] | ‘R’] | | | | | |
| LENSG00000226937.5 | ENSG00000165512 | ZNF22 | 5P | Anti | AGAGGTG | [773, | [‘F’, | [954] | [‘A’] | [‘F’] | NA | NA |
| | | | | | | 2399] | ‘F’] | | | | | |
| LENSG00000226937.5 | ENSG00000165512 | ZNF22 | 5P | Anti | GAGGTGA | [2400] | [‘F’] | [955] | [‘A’] | [‘F’] | NA | NA |
| LENSG00000226937.5 | ENSG00000165512 | ZNF22 | 5P | Anti | TGCAAAG | [967] | [‘F’] | [1177] | [‘A’] | [‘F’] | NA | NA |
| LENSG00000226937.5 | ENSG00000165512 | ZNF22 | 5P | Anti | ATTTGCC | [184] | [‘F’] | [1000] | [‘A’] | [‘F’] | NA | NA |
| LENSG00000226937.5 | ENSG00000165512 | ZNF22 | 5P | Anti | TTTGCCA | [185] | [‘F’] | [1001] | [‘A’] | [‘F’] | NA | NA |
| LENSG00000226937.5 | ENSG00000165512 | ZNF22 | 5P | Anti | CATTTGC | [183] | [‘F’] | [999] | [‘A’] | [‘F’] | NA | NA |
| LENSG00000226937.5 | ENSG00000165512 | ZNF22 | 5P | Anti | GCAGCTT | [2192, | [‘R’, | [1130] | [‘A’] | [‘F’] | NA | NA |
| | | | | | | 2353] | ‘R’] | | | | | |
| LENSG00000226937.5 | ENSG00000165512 | ZNF22 | 5P | Anti | GAAAGCA | [1492] | [‘F’] | [994] | [‘A’] | [‘F’] | NA | NA |
| LENSG00000231074.4 | ENSG00000204599 | TRIM39 | 5P | Anti | GGTTCTA | [148] | [‘F’] | [879] | [‘A’] | [‘F’] | NA | NA |
| LENSG00000272894.1 | ENSG00000164654 | MIOS | 5P | Anti | ACTGAGG | [192] | [‘R’] | [903] | [‘A’] | [‘F’] | NA | NA |
| LENSG00000255847.1 | ENSG00000187726 | DNAJB13 | 5P | Anti | CTATTTT | [1190] | [‘F’] | [883] | [‘A’] | [‘F’] | NA | NA |
| LENSG00000255847.1 | ENSG00000187726 | DNAJB13 | 5P | Anti | TTTCATT | [465] | [‘F’] | [1015] | [‘A’] | [‘F’] | NA | NA |
| LENSG00000260681.1 | ENSG00000103534 | TMC5 | 5P | Anti | TGGAGCC | [10, | [‘F’, | [1224] | [‘A’] | [‘F’] | NA | NA |
| | | | | | | 279] | ‘F’] | | | | | |
| LENSG00000137497.13 | ENSG00000184154 | LRTOMT | 5P | Anti | GGGCAGC | [234] | [‘R’] | [1128] | [‘A’] | [‘F’] | NA | NA |
| LENSG00000245482.2 | ENSG00000139133 | ALG10 | 5P | Anti | GATCAGA | [719] | [‘F’] | [1053] | [‘A’] | [‘F’] | NA | NA |
| LENSG00000245482.2 | ENSG00000139133 | ALG10 | 5P | Anti | TTGCCAA | [24, | [‘F’, | [1002] | [‘A’] | [‘F’] | NA | NA |
| | | | | | | 579] | ‘F’] | | | | | |
| LENSG00000245482.2 | ENSG00000139133 | ALG10 | 5P | Anti | CTGAAAC | [660] | [‘F’] | [1184] | [‘A’] | [‘F’] | NA | NA |
| LENSG00000250616.2 | ENSG00000090238 | YPEL3 | 5P | Anti | CGGGGGC | [50, | [‘R’, | [930] | [‘A’] | [‘F’] | NA | NA |
| | | | | | | 57] | ‘R’] | | | | | |
| LENSG00000117543.15 | ENSG00000269175 | AC093157.1 | 5P | Anti | CAAAGCA | [203] | [‘F’] | [823] | [‘A’] | [‘F’] | NA | NA |
| LENSG00000255282.2 | ENSG00000196611 | MMP1 | 5P | Anti | TTGCAAA | [15] | [‘F’] | [1176] | [‘A’] | R | NA | NA |
| LENSG00000179295.11 | ENSG00000089009 | RPL6 | 5P | Anti | CGGCGAT | [169] | [‘F’] | [1094] | [‘A’] | [‘F’] | NA | NA |
| LENSG00000205890.3 | ENSG00000162069 | CCDC64B | 5P | Anti | TCGGAGG | [39] | [‘F’] | [1037] | [‘A’] | R | NA | NA |
| LENSG00000261664.1 | ENSG00000085831 | TTC39A | 5P | Anti | AGGAGTG | [162] | [‘F’] | [1219] | [‘A’] | [‘F’] | NA | NA |
| LENSG00000245293.2 | ENSG00000155016 | CYP2U1 | 5P | Anti | GATCAGA | [1141] | [‘R’] | [1053] | [‘A’] | [‘F’] | NA | NA |
| LENSG00000245293.2 | ENSG00000155016 | CYP2U1 | 5P | Anti | GCATTTG | [952] | [‘R’] | [998] | [‘A’] | [‘F’] | NA | NA |
| LENSG00000245293.2 | ENSG00000155016 | CYP2U1 | 5P | Anti | AGCATTT | [953] | [‘R’] | [997] | [‘A’] | R | NA | NA |
| LENSG00000245293.2 | ENSG00000155016 | CYP2U1 | 5P | Anti | CATGATT | [94] | [‘R’] | [911] | [‘A’] | [‘F’] | NA | NA |
| LENSG00000245293.2 | ENSG00000155016 | CYP2U1 | 5P | Anti | ATGATTA | [93] | [‘R’] | [912] | [‘A’] | [‘F’] | NA | NA |
| LENSG00000067829.14 | ENSG00000180879 | SSR4 | 5P | Anti | GCCATGA | [131] | [‘F’] | [909] | [‘A’] | R | NA | NA |
| LENSG00000267121.1 | ENSG00000184922 | FMNL1 | 5P | Anti | AGGGCAC | [927, | [‘R’, | [1208] | [‘A’] | R | NA | NA |
| | | | | | | 1160] | ‘R’] | | | | | |
| LENSG00000267121.1 | ENSG00000184922 | FMNL1 | 5P | Anti | CTGAGGC | [701, | [‘F’, | [904] | [‘A’] | [‘F’] | NA | NA |
| | | | | | | 574, | ‘R’, | | | | | |
| | | | | | | 1665] | ‘R’] | | | | | |
| LENSG00000267121.1 | ENSG00000184922 | FMNL1 | 5P | Anti | ACTGAGG | [700] | [‘F’] | [903] | [‘A’] | [‘F’] | NA | NA |
| LENSG00000247708.3 | ENSG00000168818 | STX18 | 5P | Anti | AACTGAG | [513] | [‘F’] | [902] | [‘A’] | [‘F’] | NA | NA |
| LENSG00000259673.1 | ENSG00000189227 | C15orf61 | 5P | Anti | GTTCAAA | [536] | [‘F’] | [820] | [‘A’] | [‘F’] | NA | NA |
| LENSG00000231154.1 | ENSG00000123562 | MORF4L2 | 5P | Anti | ACCAGGA | [204] | [‘F’] | [1216] | [‘A’] | [‘F’] | NA | NA |
| LENSG00000261519.2 | ENSG00000166548 | TK2 | 5P | Anti | TGTTCAA | [43] | [‘R’] | [819] | [‘A’] | R | NA | NA |
| LENSG00000261519.2 | ENSG00000166548 | TK2 | 5P | Anti | GTTCAAA | [42] | [‘R’] | [820] | [‘A’] | [‘F’] | NA | NA |
| LENSG00000273485.1 | ENSG00000138172 | CALHM2 | 5P | Anti | TTTGCCA | [381] | [‘R’] | [1001] | [‘A’] | [‘F’] | NA | NA |
| LENSG00000236333.3 | ENSG00000072657 | TRHDE | 5P | Anti | AGCATTT | [512, | [‘F’, | [997] | [‘A’] | [‘F’] | NA | NA |
| | | | | | | 343, | ‘R’, | | | | | |
| | | | | | | 435] | ‘R’] | | | | | |
| LENSG00000265205.1 | ENSG00000109111 | SUPT6H | 5P | Anti | TGTTCAA | [103] | [‘R’] | [819] | [‘A’] | [‘F’] | NA | NA |
| LENSG00000267387.1 | ENSG00000130813 | C19orf66 | 5P | Anti | AATAGGA | [1286] | [‘F’] | [868] | [‘A’] | [‘F’] | NA | NA |
| LENSG00000267387.1 | ENSG00000130813 | C19orf66 | 5P | Anti | AGAGGTG | [902] | [‘F’] | [954] | [‘A’] | [‘F’] | NA | NA |
| LENSG00000267387.1 | ENSG00000130813 | C19orf66 | 5P | Anti | ATAGGAC | [1287] | [‘F’] | [869] | [‘A’] | [‘F’] | NA | NA |
| LENSG00000267387.1 | ENSG00000130813 | C19orf66 | 5P | Anti | ACTGAGG | [263, | [‘F’, | [903] | [‘A’] | R | NA | NA |
| | | | | | | 943, | ‘F’, | | | | | |
| | | | | | | 876, | ‘R’, | | | | | |
| | | | | | | 1252] | ‘R’] | | | | | |
| LENSG00000267387.1 | ENSG00000130813 | C19orf66 | 5P | Anti | GTGAAAT | [1269] | [‘F’] | [958] | [‘A’] | [‘F’] | NA | NA |
| LENSG00000267387.1 | ENSG00000130813 | C19orf66 | 5P | Anti | AACTGAG | [942, | [‘F’, | [902] | [‘A’] | R | NA | NA |
| | | | | | | 877, | ‘R’, | | | | | |
| | | | | | | 1253] | ‘R’] | | | | | |
| LENSG00000267387.1 | ENSG00000130813 | C19orf66 | 5P | Anti | CGCAGCT | [1217] | [‘F’] | [850] | [‘A’] | [‘F’] | NA | NA |
| LENSG00000267387.1 | ENSG00000130813 | C19orf66 | 5P | Anti | CTGAGGC | [944, | [‘F’, | [904] | [‘A’] | R | NA | NA |
| | | | | | | 875, | ‘R’, | | | | | |
| | | | | | | 1251] | ‘R’] | | | | | |
| LENSG00000267387.1 | ENSG00000130813 | C19orf66 | 5P | Anti | GTTCAAA | [1092, | [‘F’, | [820] | [‘A’] | [‘F’] | NA | NA |
| | | | | | | 1209] | ‘F’] | | | | | |
| LENSG00000267387.1 | ENSG00000130813 | C19orf66 | 5P | Anti | TAGGACC | [1288] | [‘F’] | [870] | [‘A’] | [‘F’] | NA | NA |
| LENSG00000253320.1 | ENSG00000155096 | AZIN1 | 5P | Anti | CTTTGGG | [168] | [‘F’] | [1152] | [‘A’] | [‘F’] | NA | NA |
| LENSG00000272578.1 | ENSG00000159496 | RGL4 | 5P | Anti | GAGGTTC | [206] | [‘R’] | [1040] | [‘A’] | [‘F’] | NA | NA |
| LENSG00000272578.1 | ENSG00000159496 | RGL4 | 5P | Anti | CAAGAAC | [218] | [‘R’] | [1025] | [‘A’] | [‘F’] | NA | NA |
| LENSG00000272578.1 | ENSG00000159496 | RGL4 | 5P | Anti | AGAGGTG | [114, | [‘F’, | [954] | [‘A’] | R | NA | NA |
| | | | | | | 154] | ‘R’] | | | | | |
| LENSG00000272578.1 | ENSG00000159496 | RGL4 | 5P | Anti | AGGTTCG | [205] | [‘R’] | [1041] | [‘A’] | [‘F’] | NA | NA |
| LENSG00000272578.1 | ENSG00000159496 | RGL4 | 5P | Anti | GATCATT | [234] | [‘R’] | [1861] | [‘A’] | [‘F’] | NA | NA |
| LENSG00000248724.2 | ENSG00000113971 | NPHP3 | 5P | Anti | CAAAGCT | [723, | [‘F’, | [1179] | [‘A’] | [‘F’] | NA | NA |
| | | | | | | 1548, | ‘F’, | | | | | |
| | | | | | | 14, | ‘R’, | | | | | |
| | | | | | | 163] | ‘R’] | | | | | |
| LENSG00000228775.3 | ENSG00000106028 | SSBP1 | 5P | Anti | CTATTTT | [177] | [‘R’] | [883] | [‘A’] | [‘F’] | NA | NA |
| LENSG00000234775.1 | ENSG00000163485 | ADOPA1 | 5P | Anti | GCAGGCT | [226] | [‘R’] | [1227] | [‘A’] | [‘R’] | NA | NA |
| LENSG00000186615.6 | ENSG00000126777 | KTN1 | 5P | Anti | CATTAAT | [943] | [‘R’] | [1018] | [‘A’] | [‘F’] | NA | NA |
| LENSG00000186615.6 | ENSG00000126777 | KTN1 | 5P | Anti | ATGATTA | [888] | [‘F’] | [912] | [‘A’] | R | NA | NA |
| LENSG00000186615.6 | ENSG00000126777 | KTN1 | 5P | Anti | TGCAAAG | [1080] | [‘F’] | [1177] | [‘A’] | [‘F’] | NA | NA |
| LENSG00000186615.6 | ENSG00000126777 | KTN1 | 5P | Anti | TCCCATG | [1137] | [‘F’] | [1114] | [‘A’] | [‘F’] | NA | NA |
| LENSG00000186615.6 | ENSG00000126777 | KTN1 | 5P | Anti | TTCCCAT | [1136] | [‘F’] | [1113] | [‘A’] | [‘F’] | NA | NA |
| LENSG00000228384.3 | ENSG00000258881 | AC007040.11 | 5P | Anti | CTAGGAA | [8] | [‘F’] | [855] | [‘A’] | [‘F’] | NA | NA |
| LENSG00000206417.4 | ENSG00000184897 | H1FX | 5P | Anti | CTGAGGC | [792, | [‘F’, | [904] | [‘A’] | R | NA | NA |
| | | | | | | 889, | ‘R’, | | | | | |
| | | | | | | 956, | ‘R’, | | | | | |
| | | | | | | 2563] | ‘R’] | | | | | |
| LENSG00000206417.4 | ENSG00000184897 | H1FX | 5P | Anti | AAAGCAG | [1439] | [‘F’] | [824] | [‘A’] | R | NA | NA |
| LENSG00000206417.4 | ENSG00000184897 | H1FX | 5P | Anti | AGGGCAC | [567, | [‘R’, | [1208] | [‘A’] | [‘F’] | NA | NA |
| | | | | | | 607] | ‘R’] | | | | | |
| LENSG00000228315.7 | ENSG00000159496 | RGL4 | 5P | Anti | GAGGTTC | [206] | [‘R’] | [1040] | [‘A’] | [‘F’] | NA | NA |
| LENSG00000228315.7 | ENSG00000159496 | RGL4 | 5P | Anti | CAAGAAC | [218] | [‘R’] | [1025] | [‘A’] | [‘F’] | NA | NA |
| LENSG00000228315.7 | ENSG00000159496 | RGL4 | 5P | Anti | AGAGGTG | [114, | [‘F’, | [954] | [‘A’] | R | NA | NA |
| | | | | | | 154] | ‘R’] | | | | | |
| LENSG00000228315.7 | ENSG00000159496 | RGL4 | 5P | Anti | AGGTTCG | [205] | [‘R’] | [1041] | [‘A’] | [‘F’] | NA | NA |
| LENSG00000228315.7 | ENSG00000159496 | RGL4 | 5P | Anti | GATCATT | [234] | [‘R’] | [1861] | [‘A’] | [‘F’] | NA | NA |
| LENSG00000198910.8 | ENSG00000196987 | LCA10 | 5P | Anti | GAAGGGC | [433] | [‘F’] | [1206] | [‘A’] | [‘F’] | NA | NA |
| LENSG00000198910.8 | ENSG00000196987 | LCA10 | 5P | Anti | CCGAGCC | [506] | [‘F’] | [833] | [‘A’] | [‘F’] | NA | NA |
| LENSG00000198910.8 | ENSG00000196987 | LCA10 | 5P | Anti | GGAAGGG | [432] | [‘F’] | [1205] | [‘A’] | [‘F’] | NA | NA |
| LENSG00000255808.1 | ENSG00000186642 | PDE2A | 5P | Anti | CATGACC | [4] | [‘R’] | [1117] | [‘A’] | [‘F’] | NA | NA |
| LENSG00000272405.1 | ENSG00000132692 | BCAN | 5P | Anti | GAGGTTC | [341, | [‘F’, | [1040] | [‘A’] | [‘F’] | NA | NA |
| | | | | | | 2054] | ‘F’] | | | | | |
| LENSG00000272405.1 | ENSG00000132692 | BCAN | 5P | Anti | ATGTTTT | [2023] | [‘F’] | [1011] | [‘A’] | [‘F’] | NA | NA |
| LENSG00000236778.3 | ENSG00000102786 | INTS6 | 5P | Anti | CAAAGCA | [417] | [‘F’] | [823] | [‘A’] | [‘F’] | NA | NA |
| LENSG00000224066.1 | ENSG00000160051 | IQCC | 5P | Anti | CTGAGGC | [2465] | [‘F’] | [904] | [‘A’] | [‘F’] | NA | NA |
| LENSG00000237945.3 | ENSG00000241837 | ATP5O | 5P | Anti | GGGTTCC | [11] | [‘R’] | [1156] | [‘A’] | [‘F’] | NA | NA |
| LENSG00000182841.8 | ENSG00000183569 | SERHL2 | 5P | Anti | CAGGCCC | [34] | [‘R’] | [828] | [‘A’] | [‘F’] | NA | NA |
| LENSG00000178803.6 | ENSG00000128271 | ADORA2A | 5P | Anti | CTGAGGC | [204] | [‘R’] | [904] | [‘A’] | [‘F’] | NA | NA |
| LENSG00000267092.1 | ENSG00000185761 | ADAMTSL5 | 5P | Anti | CTGAGGC | [544, | [‘F’, | [904] | [‘A’] | [‘F’] | NA | NA |
| | | | | | | 552, | ‘R’, | | | | | |
| | | | | | | 573] | ‘R’] | | | | | |
| LENSG00000245573.3 | ENSG00000176697 | BDNF | 5P | Anti | TTTCATT | [51] | [‘F’] | [1015] | [‘A’] | [‘F’] | NA | NA |
| LENSG00000268677.1 | ENSG00000224420 | ADM5 | 5P | Anti | CGAGCCG | [369] | [‘F’] | [834] | [‘A’] | [‘F’] | NA | NA |
| LENSG00000234076.1 | ENSG00000188001 | TPRG1 | 5P | Anti | CTGAGGC | [186] | [‘F’] | [904] | [‘A’] | [‘F’] | NA | NA |
| LENSG00000137133.6 | ENSG00000137103 | TMEM8B | 5P | Anti | AAGCTGA | [481] | [‘F’] | [1181] | [‘A’] | [‘F’] | NA | NA |
| LENSG00000258584.1 | ENSG00000140067 | FAM181A | 5P | Anti | GAAACCA | [172] | [‘R’] | [1141] | [‘A’] | R | NA | NA |
| LENSG00000178803.6 | ENSG00000100024 | UPB1 | 5P | Anti | CTGAGGC | [874] | [‘R’] | [904] | [‘A’] | [‘F’] | NA | NA |
| LENSG00000178803.6 | ENSG00000100024 | UPB1 | 5P | Anti | ACTGAGG | [875] | [‘R’] | [903] | [‘A’] | [‘F’] | NA | NA |
| LENSG00000221817.5 | ENSG00000166348 | USP54 | 5P | Anti | AATGATC | [3968] | [‘F’] | [1861] | [‘A’] | [‘R’] | NA | NA |
| LENSG00000221817.5 | ENSG00000166348 | USP54 | 5P | Anti | ACCAGGA | [1262] | [‘R’] | [1216] | [‘A’] | [‘F’] | NA | NA |
| LENSG00000114648.7 | ENSG00000088727 | KIF9 | 5P | Same | CAGCTTC | [59, | [‘F’, | [1131] | [‘A’] | [‘F’] | NA | NA |
| | | | | | | 1337] | ‘R’] | | | | | |
| LENSG00000114648.7 | ENSG00000088727 | KIF9 | 5P | Same | AGCTTCC | [60] | [‘F’] | [1132] | [‘A’] | [‘F’] | NA | NA |
| LENSG00000114648.7 | ENSG00000088727 | KIF9 | 5P | Same | GCAGCTT | [1338] | [‘R’] | [1130] | [‘A’] | [‘F’] | NA | NA |
| LENSG00000253320.1 | ENSG00000155096 | AZIN1 | 5P | Same | CTTTGGG | [168] | [‘F’] | [1152] | [‘A’] | [‘F’] | NA | NA |
| LENSG00000255389.1 | ENSG00000056972 | TPAF3IP2 | 5P | Same | AAGCAGG | [83, | [‘R’, | [825] | [‘A’] | [‘F’] | NA | NA |
| | | | | | | 477] | ‘R’] | | | | | |
| LENSG00000255389.1 | ENSG00000056972 | TPAF3IP2 | 5P | Same | ATGATTA | [192] | [‘F’] | [912] | [‘A’] | R | NA | NA |
| LENSG00000236364.2 | ENSG00000143179 | UCK2 | 5P | Same | ATAATGG | [580] | [‘R’] | [861] | [‘A’] | [‘F’] | NA | NA |
| LENSG00000267387.1 | ENSG00000130813 | C19orf66 | 5P | Same | AATAGGA | [1286] | [‘F’] | [868] | [‘A’] | [‘F’] | NA | NA |
| LENSG00000267387.1 | ENSG00000130813 | C19orf66 | 5P | Same | AGAGGTG | [902] | [‘F’] | [954] | [‘A’] | [‘F’] | NA | NA |
| LENSG00000267387.1 | ENSG00000130813 | C19orf66 | 5P | Same | ATAGGAC | [1287] | [‘F’] | [869] | [‘A’] | [‘F’] | NA | NA |
| LENSG00000267387.1 | ENSG00000130813 | C19orf66 | 5P | Same | ACTGAGG | [263, | [‘F’, | [903] | [‘A’] | R | NA | NA |
| | | | | | | 943, | ‘F’, | | | | | |
| | | | | | | 876, | ‘R’, | | | | | |
| | | | | | | 1252] | ‘R’] | | | | | |
| LENSG00000267387.1 | ENSG00000130813 | C19orf66 | 5P | Same | GTGAAAT | [1269] | [‘F’] | [958] | [‘A’] | [‘F’] | NA | NA |
| LENSG00000267387.1 | ENSG00000130813 | C19orf66 | 5P | Same | AACTGAG | [942, | [‘F’, | [902] | [‘A’] | R | NA | NA |
| | | | | | | 877, | ‘R’, | | | | | |
| | | | | | | 1253] | ‘R’] | | | | | |
| LENSG00000267387.1 | ENSG00000130813 | C19orf66 | 5P | Same | CGCAGCT | [1217] | [‘F’] | [850] | [‘A’] | [‘F’] | NA | NA |
| LENSG00000267387.1 | ENSG00000130813 | C19orf66 | 5P | Same | CTGAGGC | [944, | [‘F’, | [904] | [‘A’] | R | NA | NA |
| | | | | | | 875, | ‘R’, | | | | | |
| | | | | | | 1251] | ‘R’] | | | | | |
| LENSG00000267387.1 | ENSG00000130813 | C19orf66 | 5P | Same | GTTCAAA | [1092, | [‘F’, | [820] | [‘A’] | [‘F’] | NA | NA |
| | | | | | | 1209] | ‘F’] | | | | | |
| LENSG00000267387.1 | ENSG00000130813 | C19orf66 | 5P | Same | TAGGACC | [1288] | [‘F’] | [870] | [‘A’] | [‘F’] | NA | NA |
| LENSG00000261664.1 | ENSG00000085831 | TTC39A | 5P | Same | AGGAGTG | [162] | [‘F’] | [1219] | [‘A’] | [‘F’] | NA | NA |
| LENSG00000271360.1 | ENSG00000148429 | USP6NL | 5P | Same | TTTCATT | [13] | [‘R’] | [1015] | [‘A’] | [‘F’] | NA | NA |
| LENSG00000131187.5 | ENSG00000198055 | GRK6 | 5P | Same | CGCCTGG | [454] | [‘R’] | [839] | [‘A’] | [‘F’] | NA | NA |
| LENSG00000264177.1 | ENSG00000214860 | EVPLL | 5P | Same | CATTAAT | [3758, | [‘F’, | [1018] | [‘A’] | R | NA | NA |
| | | | | | | 3759] | ‘R’] | | | | | |
| LENSG00000264177.1 | ENSG00000214860 | EVPLL | 5P | Same | AAAGCAG | [3822] | [‘F’] | [824] | [‘A’] | [‘F’] | NA | NA |
| LENSG00000264177.1 | ENSG00000214860 | EVPLL | 5P | Same | AAGCAGG | [3823] | [‘F’] | [825] | [‘A’] | [‘F’] | NA | NA |
| LENSG00000264177.1 | ENSG00000214860 | EVPLL | 5P | Same | GAGGTTC | [3806] | [‘F’] | [1040] | [‘A’] | [‘F’] | NA | NA |
| LENSG00000264177.1 | ENSG00000214860 | EVPLL | 5P | Same | ATCATTA | [3621, | [‘F’, | [1862] | [‘A’] | [‘F’] | NA | NA |
| | | | | | | 3756] | ‘F’] | | | | | |
| LENSG00000264177.1 | ENSG00000214860 | EVPLL | 5P | Same | GCCAAGA | [3727] | [‘F’] | [1004] | [‘A’] | [‘F’] | NA | NA |
| LENSG00000205890.3 | ENSG00000162069 | CCDC64B | 5P | Same | TCGGAGG | [39] | [‘F’] | [1037] | [‘A’] | R | NA | NA |
| LENSG00000178803.6 | ENSG00000128271 | ADORA2A | 5P | Same | CTGAGGC | [204] | [‘R’] | [904] | [‘A’] | [‘F’] | NA | NA |
| LENSG00000231721.2 | ENSG00000128585 | MKLN1 | 5P | Same | AAAGTCT | [203] | [‘F’] | [1147] | [‘A’] | R | NA | NA |
| LENSG00000237945.3 | ENSG00000241837 | ATP5O | 5P | Same | GGGTTCC | [11] | [‘R’] | [1156] | [‘A’] | [‘F’] | NA | NA |
| LENSG00000225855.2 | ENSG00000160753 | RUSC1 | 5P | Same | AATGGAA | [469] | [‘R’] | [863] | [‘A’] | [‘F’] | NA | NA |
| LENSG00000228838.1 | ENSG00000157193 | LRP8 | 5P | Same | GGGCAGC | [54] | [‘F’] | [1128] | [‘A’] | [‘F’] | NA | NA |
| LENSG00000178803.6 | ENSG00000100024 | UPB1 | 5P | Same | CTGAGGC | [874] | [‘R’] | [904] | [‘A’] | [‘F’] | NA | NA |
| LENSG00000178803.6 | ENSG00000100024 | UPB1 | 5P | Same | ACTGAGG | [875] | [‘R’] | [903] | [‘A’] | [‘F’] | NA | NA |
| LENSG00000273485.1 | ENSG00000138172 | CALHM2 | 5P | Same | TTTGCCA | [381] | [‘R’] | [1001] | [‘A’] | [‘F’] | NA | NA |
| LENSG00000255847.1 | ENSG00000187726 | DNAJB13 | 5P | Same | CTATTTT | [1190] | [‘F’] | [883] | [‘A’] | [‘F’] | NA | NA |
| LENSG00000255847.1 | ENSG00000187726 | DNAJB13 | 5P | Same | TTTCATT | [465] | [‘F’] | [1015] | [‘A’] | [‘F’] | NA | NA |
| LENSG00000258672.1 | ENSG00000036530 | CYP46A1 | 5P | Same | GGGGGCA | [218] | [‘F’] | [931] | [‘A’] | [‘F’] | NA | NA |
| LENSG00000258672.1 | ENSG00000036530 | CYP46A1 | 5P | Same | GAATAGG | [291] | [‘F’] | [867] | [‘A’] | [‘F’] | NA | NA |
| LENSG00000258672.1 | ENSG00000036530 | CYP46A1 | 5P | Same | CCAGGAG | [207] | [‘F’] | [1217] | [‘A’] | [‘F’] | NA | NA |
| LENSG00000258672.1 | ENSG00000036530 | CYP46A1 | 5P | Same | ATGGAAT | [288] | [‘F’] | [864] | [‘A’] | [‘F’] | NA | NA |
| LENSG00000258672.1 | ENSG00000036530 | CYP46A1 | 5P | Same | GGAATAG | [290] | [‘F’] | [866] | [‘A’] | [‘F’] | NA | NA |
| LENSG00000258672.1 | ENSG00000036530 | CYP46A1 | 5P | Same | TGGAATA | [289] | [‘F’] | [865] | [‘A’] | [‘F’] | NA | NA |
| LENSG00000258672.1 | ENSG00000036530 | CYP46A1 | 5P | Same | GCAGGCC | [266] | [‘F’] | [827] | [‘A’] | [‘F’] | NA | NA |
| LENSG00000114529.8 | ENSG00000174500 | GCSAM | 5P | Same | CTGAGGC | [251] | [‘F’] | [904] | [‘A’] | [‘F’] | NA | NA |
| LENSG00000257097.1 | ENSG00000130779 | CLIP1 | 5P | Same | TTTGGGT | [644] | [‘F’] | [1153] | [‘A’] | [‘F’] | NA | NA |
| LENSG00000226937.5 | ENSG00000165512 | ZNF22 | 5P | Same | AGTGTTC | [2433, | [‘F’, | [817] | [‘A’] | R | NA | NA |
| | | | | | | 2109] | ‘R’] | | | | | |
| LENSG00000226937.5 | ENSG00000165512 | ZNF22 | 5P | Same | AGAGGTG | [773, | [‘F’, | [954] | [‘A’] | [‘F’] | NA | NA |
| | | | | | | 2399] | ‘F’] | | | | | |
| LENSG00000226937.5 | ENSG00000165512 | ZNF22 | 5P | Same | GAGGTGA | [2400] | [‘F’] | [955] | [‘A’] | [‘F’] | NA | NA |
| LENSG00000226937.5 | ENSG00000165512 | ZNF22 | 5P | Same | TGCAAAG | [967] | [‘F’] | [1177] | [‘A’] | [‘F’] | NA | NA |
| LENSG00000226937.5 | ENSG00000165512 | ZNF22 | 5P | Same | ATTTGCC | [184] | [‘F’] | [1000] | [‘A’] | [‘F’] | NA | NA |
| LENSG00000226937.5 | ENSG00000165512 | ZNF22 | 5P | Same | TTTGCCA | [185] | [‘F’] | [1001] | [‘A’] | [‘F’] | NA | NA |
| LENSG00000226937.5 | ENSG00000165512 | ZNF22 | 5P | Same | CATTTGC | [183] | [‘F’] | [999] | [‘A’] | [‘F’] | NA | NA |
| LENSG00000226937.5 | ENSG00000165512 | ZNF22 | 5P | Same | GCAGCTT | [2192, | [‘R’, | [1130] | [‘A’] | [‘F’] | NA | NA |
| | | | | | | 2353] | ‘R’] | | | | | |
| LENSG00000226937.5 | ENSG00000165512 | ZNF22 | 5P | Same | GAAAGCA | [1492] | [‘F’] | [994] | [‘A’] | [‘F’] | NA | NA |
| LENSG00000204934.6 | ENSG00000171130 | ATP6V0E2 | 5P | Same | GAAAGCA | [114, | [‘F’, | [994] | [‘A’] | [‘F’] | NA | NA |
| | | | | | | 179] | ‘R’] | | | | | |
| LENSG00000255119.1 | ENSG00000184224 | C11orf72 | 5P | Same | CAGCTTC | [499] | [‘F’] | [1131] | [‘A’] | [‘F’] | NA | NA |
| LENSG00000107872.8 | ENSG00000059915 | PSD | 5P | Same | AAGGAAT | [238] | [‘R’] | [1194] | [‘A’] | [‘F’] | NA | NA |
| LENSG00000258584.1 | ENSG00000140067 | FAM181A | 5P | Same | GAAACCA | [172] | [‘R’] | [1141] | [‘A’] | R | NA | NA |
| LENSG00000259673.1 | ENSG00000189227 | C15orf61 | 5P | Same | GTTCAAA | [536] | [‘F’] | [820] | [‘A’] | [‘F’] | NA | NA |
| LENSG00000231154.1 | ENSG00000123562 | MORF4L2 | 5P | Same | ACCAGGA | [204] | [‘F’] | [1216] | [‘A’] | [‘F’] | NA | NA |
| LENSG00000231074.4 | ENSG00000204599 | TRIM39 | 5P | Same | GGTTCTA | [148] | [‘F’] | [879] | [‘A’] | [‘F’] | NA | NA |
| LENSG00000271122.1 | ENSG00000122557 | HERPUD2 | 5P | Same | AAGCTGA | [235] | [‘F’] | [1181] | [‘A’] | [‘F’] | NA | NA |
| LENSG00000138018.13 | ENSG00000173567 | GPR113 | 5P | Same | GAGTGGA | [91] | [‘F’] | [1221] | [‘A’] | R | NA | NA |
| LENSG00000236295.1 | ENSG00000223874 | AC007557.1 | 5P | Same | AAGCAGG | [358] | [‘F’] | [825] | [‘A’] | [‘F’] | NA | NA |
| LENSG00000257176.1 | ENSG00000064763 | FAR2 | 5P | Same | GGATCAT | [178] | [‘R’] | [1860] | [‘A’] | [‘F’] | NA | NA |
| LENSG00000137497.13 | ENSG00000184154 | LRTOMT | 5P | Same | GGGCAGC | [234] | [‘R’] | [1128] | [‘A’] | [‘F’] | NA | NA |
| LENSG00000204261.4 | ENSG00000204264 | PSMB8 | 5P | Same | GCAGCTT | [281, | [‘R’, | [1130] | [‘A’] | [‘F’] | NA | NA |
| | | | | | | 1088] | ‘R’] | | | | | |
| LENSG00000255282.2 | ENSG00000196611 | MMP1 | 5P | Same | TTGCAAA | [15] | [‘F’] | [1176] | [‘A’] | R | NA | NA |
| LENSG00000272578.1 | ENSG00000159496 | RGL4 | 5P | Same | GAGGTTC | [206] | [‘R’] | [1040] | [‘A’] | [‘F’] | NA | NA |
| LENSG00000272578.1 | ENSG00000159496 | RGL4 | 5P | Same | CAAGAAC | [218] | [‘R’] | [1025] | [‘A’] | [‘F’] | NA | NA |
| LENSG00000272578.1 | ENSG00000159496 | RGL4 | 5P | Same | AGAGGTG | [114, | [‘F’, | [954] | [‘A’] | R | NA | NA |
| | | | | | | 154] | ‘R’] | | | | | |
| LENSG00000272578.1 | ENSG00000159496 | RGL4 | 5P | Same | AGGTTCG | [205] | [‘R’] | [1041] | [‘A’] | [‘F’] | NA | NA |
| LENSG00000272578.1 | ENSG00000159496 | RGL4 | 5P | Same | GATCATT | [234] | [‘R’] | [1861] | [‘A’] | [‘F’] | NA | NA |
| LENSG00000161040.12 | ENSG00000128606 | LRRC17 | 5P | Same | AATCAAG | [286] | [‘F’] | [1022] | [‘A’] | [‘F’] | NA | NA |
| LENSG00000161040.12 | ENSG00000128606 | LRRC17 | 5P | Same | GGATACC | [355] | [‘F’] | [844] | [‘A’] | [‘F’] | NA | NA |
| LENSG00000161040.12 | ENSG00000128606 | LRRC17 | 5P | Same | TTGTTGG | [370] | [‘F’] | [888] | [‘A’] | [‘F’] | NA | NA |
| LENSG00000161040.12 | ENSG00000128606 | LRRC17 | 5P | Same | ACGGACC | [346] | [‘F’] | [982] | [‘A’] | [‘F’] | NA | NA |
| LENSG00000266962.1 | ENSG00000108786 | HSD17B1 | 5P | Same | AGCTTCC | [648] | [‘F’] | [1132] | [‘A’] | [‘F’] | NA | NA |
| LENSG00000266962.1 | ENSG00000108786 | HSD17B1 | 5P | Same | GCAGCTA | [677] | [‘F’] | [851] | [‘A’] | [‘F’] | NA | NA |
| LENSG00000266962.1 | ENSG00000108786 | HSD17B1 | 5P | Same | AGCTAGG | [679, | [‘F’, | [853] | [‘A’] | R | NA | NA |
| | | | | | | 2221] | ‘R’] | | | | | |
| LENSG00000266962.1 | ENSG00000108786 | HSD17B1 | 5P | Same | CAGCTAG | [678] | [‘F’] | [852] | [‘A’] | [‘F’] | NA | NA |
| LENSG00000228315.7 | ENSG00000159496 | RGL4 | 5P | Same | GAGGTTC | [206] | [‘R’] | [1040] | [‘A’] | [‘F’] | NA | NA |
| LENSG00000228315.7 | ENSG00000159496 | RGL4 | 5P | Same | CAAGAAC | [218] | [‘R’] | [1025] | [‘A’] | [‘F’] | NA | NA |
| LENSG00000228315.7 | ENSG00000159496 | RGL4 | 5P | Same | AGAGGTG | [114, | [‘F’, | [954] | [‘A’] | R | NA | NA |
| | | | | | | 154] | ‘R’] | | | | | |
| LENSG00000228315.7 | ENSG00000159496 | RGL4 | 5P | Same | AGGTTCG | [205] | [‘R’] | [1041] | [‘A’] | [‘F’] | NA | NA |
| LENSG00000228315.7 | ENSG00000159496 | RGL4 | 5P | Same | GATCATT | [234] | [‘R’] | [1861] | [‘A’] | [‘F’] | NA | NA |
| LENSG00000100911.9 | ENSG00000092098 | RNF31 | 5P | Same | TCTTGGA | [389] | [‘R’] | [965] | [‘A’] | [‘F’] | NA | NA |
| LENSG00000186615.6 | ENSG00000126777 | KTN1 | 5P | Same | CATTAAT | [943] | [‘R’] | [1018] | [‘A’] | [‘F’] | NA | NA |
| LENSG00000186615.6 | ENSG00000126777 | KTN1 | 5P | Same | ATGATTA | [888] | [‘F’] | [912] | [‘A’] | R | NA | NA |
| LENSG00000186615.6 | ENSG00000126777 | KTN1 | 5P | Same | TGCAAAG | [1080] | [‘F’] | [1177] | [‘A’] | [‘F’] | NA | NA |
| LENSG00000186615.6 | ENSG00000126777 | KTN1 | 5P | Same | TCCCATG | [1137] | [‘F’] | [1114] | [‘A’] | [‘F’] | NA | NA |
| LENSG00000186615.6 | ENSG00000126777 | KTN1 | 5P | Same | TTCCCAT | [1136] | [‘F’] | [1113] | [‘A’] | [‘F’] | NA | NA |
| LENSG00000247708.3 | ENSG00000168818 | STX18 | 5P | Same | AACTGAG | [513] | [‘F’] | [902] | [‘A’] | [‘F’] | NA | NA |
| LENSG00000266304.1 | ENSG00000266258 | RP11- | 5P | Same | AACTTAA | [305] | [‘F’] | [1188] | [‘A’] | [‘F’] | NA | NA |
| | | 41O4.1 | | | | | | | | | | |
| LENSG00000266304.1 | ENSG00000266258 | RP11- | 5P | Same | GAGGCCA | [292] | [‘F’] | [906] | [‘A’] | [‘F’] | NA | NA |
| | | 41O4.1 | | | | | | | | | | |
| LENSG00000245293.2 | ENSG00000155016 | CYP2U1 | 5P | Same | GATCAGA | [1141] | [‘R’] | [1053] | [‘A’] | [‘F’] | NA | NA |
| LENSG00000245293.2 | ENSG00000155016 | CYP2U1 | 5P | Same | GCATTTG | [952] | [‘R’] | [998] | [‘A’] | [‘F’] | NA | NA |
| LENSG00000245293.2 | ENSG00000155016 | CYP2U1 | 5P | Same | AGCATTT | [953] | [‘R’] | [997] | [‘A’] | R | NA | NA |
| LENSG00000245293.2 | ENSG00000155016 | CYP2U1 | 5P | Same | CATGATT | [94] | [‘R’] | [911] | [‘A’] | [‘F’] | NA | NA |
| LENSG00000245293.2 | ENSG00000155016 | CYP2U1 | 5P | Same | ATGATTA | [93] | [‘R’] | [912] | [‘A’] | [‘F’] | NA | NA |
| LENSG00000226377.1 | ENSG00000176658 | MYO1D | 5P | Same | GGTTGCA | [634] | [‘F’] | [1174] | [‘A’] | [‘F’] | NA | NA |
| LENSG00000255808.1 | ENSG00000186642 | PDE2A | 5P | Same | CATGACC | [4] | [‘R’] | [1117] | [‘A’] | [‘F’] | NA | NA |
| LENSG00000228775.3 | ENSG00000106028 | SSBP1 | 5P | Same | CTATTTT | [177] | [‘R’] | [883] | [‘A’] | [‘F’] | NA | NA |
| LENSG00000250616.2 | ENSG00000090238 | YPEL3 | 5P | Anti | CCGGGGG | [51] | [‘R’] | [262, | [‘I’, | [‘F’, | NA | NA |
| | | | | | | | | 929, | ‘A’, | ‘F’, | | |
| | | | | | | | | 1161] | ‘A’] | ‘F’] | | |
| LENSG00000161040.12 | ENSG00000128606 | LRRC17 | 5P | Same | CCGGGGG | [33, | [‘F’, | [262, | [‘I’, | [‘F’, | NA | NA |
| | | | | | | 423] | ‘F’] | 929, | ‘A’, | ‘F’, | | |
| | | | | | | | | 1161] | ‘A’] | ‘F’] | | |
| LENSG00000228384.3 | ENSG00000144043 | TEX261 | 5P | Anti | GCCGGGG | [3539] | [‘F’] | [261, | [‘I’, | [‘F’, | NA | NA |
| | | | | | | | | 928, | ‘A’, | ‘F’, | | |
| | | | | | | | | 1753, | ‘I’, | ‘F’, | | |
| | | | | | | | | 256] | ‘I’] | ‘R’] | | |
| LENSG00000230487.3 | ENSG00000157778 | PSMG3 | 5P | Anti | AGAGGGA | [407] | [‘R’] | [432, | [‘I’, | R | NA | NA |
| | | | | | | | | 919, | ‘A’, | | | |
| | | | | | | | | 1445] | ‘I’] | | | |
| LENSG00000230487.3 | ENSG00000157778 | PSMG3 | 5P | Same | AGAGGGA | [407] | [‘R’] | [432, | [‘I’, | R | NA | NA |
| | | | | | | | | 919, | ‘A’, | | | |
| | | | | | | | | 1445] | ‘I’] | | | |
| LENSG00000255389.1 | ENSG00000056972 | TRAF3IP2 | 5P | Anti | GAGCCTG | [486] | [‘F’] | [437, | [‘I’, | R | NA | NA |
| | | | | | | | | 1226] | ‘A’] | | | |
| LENSG00000245482.2 | ENSG00000139133 | ALG10 | 5P | Anti | TGAAACT | [661] | [‘F’] | [80, | [‘I’, | [‘F’, | NA | NA |
| | | | | | | | | 1185] | ‘A’] | ‘F’] | | |
| LENSG00000245482.2 | ENSG00000139133 | ALG10 | 5P | Anti | ATCAGAT | [720] | [‘F’] | [220, | [‘I’, | [‘F’, | NA | NA |
| | | | | | | | | 1054] | ‘A’] | ‘F’] | | |
| LENSG00000231154.1 | ENSG00000123562 | MORF4L2 | 5P | Anti | TGATCCT | [275, | [‘F’, | [8, | [‘I’, | [‘F’, | NA | NA |
| | | | | | | 261] | ‘R’] | 1859] | ‘A’] | ‘R’] | | |
| LENSG00000255389.1 | ENSG00000056972 | TRAF3IP2 | 5P | Same | GAGCCTG | [486] | [‘F’] | [437, | [‘I’, | R | NA | NA |
| | | | | | | | | 1226] | ‘A’] | | | |
| LENSG00000188735.8 | ENSG00000139725 | RHOF | 5P | Same | CGGCGGC | [179] | [‘R’] | [277, | [‘I’, | [‘F’, | NA | NA |
| | | | | | | | | 1102] | ‘A’] | ‘F’] | | |
| LENSG00000264177.1 | ENSG00000214860 | EVPLL | 5P | Same | TCATTAA | [3757] | [‘F’] | [96, | [‘I’, | [‘F’, | NA | NA |
| | | | | | | | | 1017] | ‘A’] | ‘F’] | | |
| LENSG00000244198.1 | ENSG00000213214 | ARHGEF35 | 5P | Same | GGAGCCT | [581] | [‘R’] | [436, | [‘I’, | [‘F’, | NA | NA |
| | | | | | | | | 1225] | ‘A’] | ‘F’] | | |
| LENSG00000258672.1 | ENSG00000036530 | CYP46A1 | 5P | Same | AGCAGGC | [265] | [‘F’] | [472, | [‘I’, | [‘F’, | NA | NA |
| | | | | | | | | 826] | ‘A’] | ‘F’] | | |
| LENSG00000231154.1 | ENSG00000123562 | MORF4L2 | 5P | Same | TGATCCT | [275, | [‘F’, | [8, | [‘I’, | [‘F’, | NA | NA |
| | | | | | | 261] | ‘R’] | 1859] | ‘A’] | ‘R’] | | |

### Summary of Foregoing Findings

The foregoing description discloses data demonstrating the two important sequence components of t-NAT1 and t-NAT2:
(1) The MIR element in the 3′ end of both transcripts of MAPT AS-lncRNA, t-NAT1 and t-NAT2 (e.g. see FIGS. 1, 2, 6 and 7)
(2) The region of 5′-5′ antisense overlap of t-NAT1 with the 5′UTR of MAPT. (e.g. see FIGS. 1 and 2)

This indicates that the region of 5′-5′ antisense overlap confers specificity to the host coding gene (e.g. MAPT), whereas the MIR element is responsible for a generalised and conserved role in repressing translation, which may apply to other MIR-containing lncRNAs (MIR-lncRNAs) that are paired in antisense orientation with protein coding genes.

Using ribosomal profiling, we demonstrated that t-NAT1 and t-NAT2 mediate translational regulation of tau by influencing recruitment to the ribosome; and the MIR element is essential for this activity (see FIG. 18 and also FIG. 22).

Using bioinformatics analysis, we also identified the two 7-mer motifs in the conserved CORE-SINE region of the MIR element that are described above e.g. as underlined in SEQ IDs 1-3 on Page 17 and as mentioned on Page 19. See also FIG. 22, wherein the two motifs are numbered Motif 1 (CACCCAC) and Motif 2 (CUGAGGC) and wherein the relative position within the MIR is indicated as dark boxes (in the lower left panel of FIG. 22).

### Further Data Obtained Using MiniNAT Sequences

(1) MiniNAT:

Having shown that the MIR element, shared by t-NAT1 and t-NAT2, and the 5′ region of t-NAT1 that overlaps in antisense orientation with some of the 5′-UTR of MAPT (region of overlap) confer activity of the t-NATs, the inventors went on to show that the region of overlap and the MIR element alone are sufficient for a therapeutic RNA to retain the capacity to specifically repress tau translation. Advantageously, shorter sequences may be more amenable to therapeutic delivery to CNS.

To test this, a vector for the expression of the MiniNAT for t-NAT1, effectively reducing sequence length from 449 bp to 94 bp (see FIG. 22, lowermost panel).

In stably expressing cell lines (SH-SY5Y human neuroblastoma), it was demonstrated that the MiniNAT retained capacity to repress tau protein levels, even more effectively than the full-length t-NAT1 (lowermost panel of FIG. 22).

(1) 7-Mer Motifs:

To show that the 7mer motifs, Motif 1 and Motif 2, are able to form a functional MIR-lncRNA, we also expressed the full-length t-NAT1 but with each of the motifs deleted. Removal of either Motif 1 or Motif 2, but not Motif 3, abolished the repressive capacity of t-NAT1 (see the lower panel of FIG. 22).

### In Vivo Validation

To further validate previous findings in vivo and confirm the therapeutic benefits of tau reduction, the inventors are extending the study to mouse models. Firstly, the htau mouse model, which carries the full-length human MAPT against a Mapt (−/−) background. This is the only suitable mouse model as the transgene includes the human MAPT promoter and first non-coding exon spanning the core promoter and region of overlap. The inventors are also generating the mouse equivalent of a miniNAT, or optimised mNAT (mouse-NAT) to test against the endogenous Mapt gene in non-transgenic mice.

The miniNATs or mNATs will be delivered by direct brain injection of AAV9 vector in mouse pups (P1). Translational repression will then be analysed 1-4 weeks later by quantifying transcript and protein levels. Toxin-induced seizures in a mouse model of epilepsy will be quantified. It has been previously shown that reduction of tau levels results in reductions in seizures in chemically treated mice and also in APP mouse models. The effect of tau reduction in AAV9-t-NAT treated tauopathy mice (htau) on pathological progression and behavioural deficits will also be determined.

### Conclusion

The t-NAT1 sequence can be reduced to the following elements; the MIR domain and the 5′ region of antisense overlap with the MAPT 5′-UTR, without loss of function.

Removal of repressive function with deletion of 7-mer motifs supports a role of MAPT-AS1 MIR-lncRNA in suppressing recruitment of MAPT mRNA to the 40S ribosomal subunit through a competitive pairing with the 18S rRNA mediated by the MIR Motif 1 and Motif 2.

### Methods References:

### Vienna Package

Lorenz, Ronny and Bernhart, Stephan H. and Honer zu Siederdissen, Christian and Tafer, Hakim and Flamm, Christoph and Stadler, Peter F. and Hofacker, Ivo L. ViennaRNA Package 2.0 Algorithms for Molecular Biology, 6:1 26, 2011, doi:10.1186/1748-7188-6-26

### Python

Python Software Foundation. Python Language Reference, version 2.7. Available at http://www.python.org

### R

R Core Team (2014). R: A language and environment for statistical computing. R Foundation for Statistical Computing, Vienna, Austria. URL (http://www.R-project.org/

### Ggplot2

H. Wickham. ggplot2: elegant graphics for data analysis. Springer New York, 2009.

### Dplyr+tidyr

Hadley Wickham (2014). tidyr: Easily Tidy Data with spread( ) and gather( ) Functions. R package version 0.2.0. http://CRAN.R-project.org/package=tidyr
Hadley Wickham and Romain Francois (2015). dplyr: A Grammar of Data Manipulation. R package version 0.4.1. http://CRAN.R-project.org/package=dplyr

### Bedtools (v2.22.1)

http://bioinformatics.oxfordjournals.org/content/26/6/841.short

### REFERENCES

All publications, patent and patent applications cited herein or filed with this application, including references filed as part of an Information Disclosure Statement are incorporated by reference in their entirety.
1. ENCODE Project Consortium. An integrated encyclopedia of DNA elements in the human genome. Nature 489, 57-74 (2012).
2. Carninci, P. et al. The transcriptional landscape of the mammalian genome. Science 309, 1559-1563 (2005).
3. Djebali, S. et al. Landscape of transcription in human cells. Nature 489, 101-108 (2012).
4. Rinn, J. L. & Chang, H. Y. Genome regulation by long noncoding RNAs. Annu. Rev. Biochem. 81, 145-166 (2012).
5. Amaral, P. P., Dinger, M. E. & Mattick, J. S. Non-coding RNAs in homeostasis, disease and stress responses: an evolutionary perspective. Brief. Funct. Genomics 12, 254-278 (2013).
6. Quinn, J. J. & Chang, H. Y. Unique features of long non-coding RNA biogenesis and function. Nat. Rev. Genet. 17, 47-62 (2015).
7. Spillantini, M. G. & Goedert, M. Tau pathology and neurodegeneration. Lancet Neurol. 12, 609-622 (2013).
8. Baker, M. et al. Association of an extended haplotype in the tau gene with progressive supranuclear palsy. Hum. Mol. Genet. 8, 711-715 (1999).
9. Houlden, H. et al. Corticobasal degeneration and progressive supranuclear palsy share a common tau haplotype. Neurology 56, 1702-1706 (2001).
10. Desikan, R. S. et al. Genetic overlap between Alzheimer's disease and Parkinson's disease at the MAPT locus. Mol.

Psychiatry 20, 1588-1595 (2015).
11. Santacruz, K. et al. Tau suppression in a neurodegenerative mouse model improves memory function. Science 309, 476-481 (2005).
12. Vossel, K. A. et al. Tau reduction prevents Abeta-induced defects in axonal transport. Science 330, 198 (2010).
13. Roberson, E. D. et al. Reducing endogenous tau ameliorates amyloid beta-induced deficits in an Alzheimer's disease mouse model. Science 316, 750-754 (2007).
14. Pittman, A. M. et al. Linkage disequilibrium fine mapping and haplotype association analysis of the tau gene in progressive supranuclear palsy and corticobasal degeneration. J. Med. Genet. 42, 837-846 (2005).
15. Lizio, M. et al. Gateways to the FANTOM5 promoter level mammalian expression atlas. Genome Biol. 16, 22 (2015).
16. Plessy, C. et al. Linking promoters to functional transcripts in small samples with nanoCAGE and CAGEscan. Nat. Methods 7, 528-534 (2010).
17. Lin, M. F., Jungreis, I. & Kellis, M. PhyloCSF: a comparative genomics method to distinguish protein coding and non-coding regions. Bioinforma. Oxf. Engl. 27, i275-282 (2011).
18. Glazko, G. V. & Nei, M. Estimation of divergence times for major lineages of primate species. Mol. Biol. Evol. 20, 424-434 (2003).
19. Gilbert, N. & Labuda, D. CORE-SINEs: eukaryotic short interspersed retroposing elements with common sequence motifs. Proc. Natl. Acad. Sci. U.S.A 96, 2869-2874 (1999).
20. Sibley, C. R. et al. Recursive splicing in long vertebrate genes. Nature 521, 371-375 (2015).
21. Morita, T. & Sobue, K. Specification of neuronal polarity regulated by local translation of CRMP2 and Tau via the mTOR-p 70S6K pathway. J. Biol. Chem. 284, 27734-27745 (2009).
22. Veo, B. L. & Krushel, L. A. Secondary RNA structure and nucleotide specificity contribute to internal initiation mediated by the human tau 5′ leader. RNA Biol. 9, 1344-1360 (2012).
23. Mauro, V. P. & Edelman, G. M. The ribosome filter hypothesis. Proc. Natl. Acad. Sci. U.S.A 99, 12031-12036 (2002).
24. Pánek, J., Kolár, M., Vohradský, J. & Shivaya Valásek, L. An evolutionary conserved pattern of 18S rRNA sequence complementarity to mRNA 5′ UTRs and its implications for eukaryotic gene translation regulation. Nucleic Acids Res. 41, 7625-7634 (2013).
25. Weingarten-Gabbay, S. et al. Comparative genetics. Systematic discovery of cap-independent translation sequences in human and viral genomes. Science 351, (2016).
26. Kapusta, A. et al. Transposable elements are major contributors to the origin, diversification, and regulation of vertebrate long noncoding RNAs. PLoS Genet. 9, e1003470 (2013).
27. Chen, E. Y. et al. Enrichr: interactive and collaborative HTML5 gene list enrichment analysis tool. BMC Bioinformatics 14, 128 (2013).
28. Xia, J., Benner, M. J. & Hancock, R. E. W. NetworkAnalyst—integrative approaches for protein-protein interaction network analysis and visual exploration. Nucleic Acids Res. 42, W167-174 (2014).
29. Jjingo, D. et al. Mammalian-wide interspersed repeat (MIR)-derived enhancers and the regulation of human gene expression. Mob. DNA 5, 14 (2014).
30. Wang, J. et al. MIR retrotransposon sequences provide insulators to the human genome. Proc. Natl. Acad. Sci. U.S.A 112, E4428-4437 (2015).
31. Sposito, T. et al. Developmental regulation of tau splicing is disrupted in stem cell-derived neurons from frontotemporal dementia patients with the 10+16 splice-site mutation in MAPT. Hum. Mol. Genet. 24, 5260-5269 (2015).
32. Potter, C. J. & Luo, L. Splinkerette PCR for mapping transposable elements in Drosophila. PloS One 5, e10168 (2010).
33. Boris Kantor, Rachel M. Bailey, Keon Wimberly, Sahana N. Kalburgi, Steven J. Gray, Advances in Genetics, Volume 87 (2014).
34. Marc S. Weinberg a, R. Jude Samulski a,b, Thomas J. McCowna, Neuropharmacology 69 (2013) pp82-88.
35. Petrov AS, Bernier C R, Gulen B, Waterbury C C, Hershkovits E, Hsiao C, Harvey S C, Hud N V, Fox G E, Wartell R M, Williams L D. Secondary structures of rRNAs from all three domains of life. PLoS One. 2014 Feb. 5; 9(2):e88222

1. A vector for delivering to a cell, or expressing in a cell, a therapeutic RNA,
wherein the therapeutic RNA is capable of reducing expression of a target gene,
wherein the therapeutic RNA comprises one or more nucleotide sequences that correspond with an antisense long non-coding RNA (AS-lncRNA), wherein the AS-lncRNA comprises a MIR domain in inverse orientation and wherein the AS-lncRNA is encoded by a genomic DNA sequence that is antisense to the target gene, and
wherein the therapeutic RNA comprises a sequence that corresponds with the MIR domain.

2. The vector according to claim 1, wherein the genomic sequence encoding the AS-lncRNA comprises an exon at the 5′ end of the AS-lncRNA that overlaps with the target gene and wherein the therapeutic RNA comprises a nucleotide sequence that corresponds with the exon at the 5′ end of the AS-lncRNA.

3. The vector according to claim 2, wherein the exon at the 5′ end of the AS-lncRNA overlaps at least partially with the 5′ UTR of the target gene.

4. The vector according to claim 2 or claim 3, wherein the exon at the 5′ end of the AS-lncRNA overlaps at least partially with an intron of the target gene.

5. The vector according to any preceding claim, wherein the sequence of the therapeutic RNA that corresponds with the MIR domain comprises a nucleotide sequence having at least 70% identity to a portion of the MIR domain of any one of SEQ ID NOs: 1-8 that is able to drive repression of target gene expression, wherein sequence identity is determined across the full length of the portion.

6. The vector according to any preceding claim, wherein the sequence of the therapeutic RNA that corresponds with the MIR domain comprises a ‘CACCCAC’ and/or a ‘CTGAGGC’ motif.

7. The vector according to any preceding claim, wherein the target gene encodes tau protein.

8. A vector for delivering to a cell, or expressing in a cell, a therapeutic RNA,
wherein the therapeutic RNA is capable of enhancing expression of a target gene,
wherein the therapeutic RNA comprises one or more nucleotide sequences that correspond with an antisense long non-coding RNA (AS-lncRNA), wherein the AS-lncRNA comprises a MIR domain in direct orientation and wherein the AS-lncRNA is encoded by a genomic DNA sequence that is antisense to the target gene, and
wherein the therapeutic RNA comprises a sequence that corresponds with the MIR domain.

9. The vector according to claim 8, wherein the genomic sequence encoding the AS-lncRNA comprises an exon at the 5′ end of the AS-lncRNA that overlaps with the target gene and wherein the therapeutic RNA comprises a nucleotide sequence that corresponds with the exon at the 5′ end of the AS-lncRNA.

10. The vector according to claim 9, wherein the exon at the 5′ end of the AS-lncRNA overlaps at least partially with the 5′ UTR of the target gene.

11. The vector according to claim 10, wherein the exon at the 5′ end of the AS-lncRNA overlaps at least partially with an intron of the target gene.

12. The vector according to any one of claims 8-11, wherein the sequence of the therapeutic RNA that corresponds with the MIR domain comprises a nucleotide sequence having at least 70% identity to a portion of the MIR domain of SEQ ID NO: 9 or 10 that is able to drive enhancement of expression of the target gene, wherein sequence identity is determined across the full length of the portion.

13. The vector according to any preceding claim, wherein the sequence of the therapeutic RNA that corresponds with the MIR domain comprises a ‘CACCCAC’ and/or a ‘CUGAGGC’ motif.

14. The vector according to any preceding claim, wherein the target gene is selected from the group consisting of the target genes listed in Table 1.

15. The vector according to any preceding claim, wherein the vector comprises a cDNA which encodes the therapeutic RNA.

16. The vector according to claim 15, wherein the vector is a plasmid vector.

17. The vector according to claim 15, wherein the vector is an AAV vector.

18. The vector according to any one of claims 1-14, wherein the vector comprises the therapeutic RNA.

19. The vector according claim 18, wherein the vector is a nanoparticle, a dendrimer, a polyplex, a liposome, a micelle or a lipoplex.

20. The vector according claim 16, wherein the plasmid vector is associated with a nanoparticle, a dendrimer, a polyplex, a liposome, a micelle or a lipoplex.

21. The vector according to any preceding claim for use in a method of treating the human or animal body by therapy.

22. The vector according to any preceding claim for use in a method of treating a neurodegenerative condition in a subject, the method comprising the administration of the vector to the subject.

23. The vector for the use according to claim 22, wherein the neurodegenerative condition is a tauopathy.

24. The vector for the use according to claim 23, wherein the tauopathy is Alzheimer's disease.

25. The vector for the use according to claim 22 or claim 23, wherein the neurodegenerative condition is Parkinson's disease.

26. A therapeutic RNA, wherein the therapeutic RNA is capable of reducing expression of a target gene,
wherein the therapeutic RNA comprises one or more nucleotide sequences that correspond with an antisense long non-coding RNA (AS-lncRNA), wherein the AS-lncRNA comprises a MIR domain in inverse orientation and wherein the AS-lncRNA is encoded by a genomic DNA sequence that is antisense to the target gene, and
wherein the therapeutic RNA comprises a sequence that corresponds with the MIR domain.

27. The therapeutic RNA according to claim 26, wherein the target gene encodes tau protein.

28. A therapeutic RNA, wherein the therapeutic RNA is capable of enhancing expression of a target gene,
wherein the therapeutic RNA comprises one or more nucleotide sequences that correspond with an antisense long non-coding RNA (AS-lncRNA), wherein the AS-lncRNA comprises a MIR domain in direct orientation and wherein the AS-lncRNA is encoded by a genomic DNA sequence that is antisense to the target gene, and
wherein the therapeutic RNA comprises a sequence that corresponds with the MIR domain.

29. The therapeutic RNA according to any one of claims 26-28, wherein the genomic sequence encoding the AS-lncRNA comprises an exon at the 5′ end of the AS-lncRNA that overlaps with the target gene and wherein the therapeutic RNA comprises a nucleotide sequence that corresponds with the exon at the 5′ end of the AS-lncRNA.

30. The therapeutic RNA according to claim 29, wherein the exon at the 5′ end of the AS-lncRNA overlaps at least partially with the 5′ UTR of the target gene.

31. The therapeutic RNA according to claim 29 or claim 30, wherein the exon at the 5′ end of the AS-lncRNA overlaps at least partially with an intron of the target gene.

32. The therapeutic RNA according to any one of claims 26-31, wherein the therapeutic RNA comprises a nucleotide sequence having at least 70% identity to a portion of the MIR domain of any one of SEQ ID NOs: 1-10 that is able to drive modulation of expression of the target gene, wherein sequence identity is determined across the full length of the portion.

33. The therapeutic RNA according to any one of claims 26-32, wherein the sequence of the therapeutic RNA that corresponds with the MIR domain comprises a ‘CACCCAC’ and/or a ‘CUGAGGC’ motif.

34. The therapeutic RNA according to any one of claims 26-33 for use in a method of treating the human or animal body by therapy.

35. The therapeutic RNA according to any one of claims 26-33 for use in a method of treating a neurodegenerative condition in a subject, the method comprising the administration of the vector to the subject.

36. The therapeutic RNA for the use according to claim 35, wherein the neurodegenerative condition is a tauopathy.

37. The therapeutic RNA for the use according to claim 36, wherein the tauopathy is Alzheimer's disease.

38. The therapeutic RNA for the use according to claim 35 or claim 36, wherein the neurodegenerative condition is Parkinson's disease.

39. A method of producing a genetically engineered organism, the method comprising introducing the MAPT-AS1 gene into one or more cells of an organism to produce the genetically engineered organism.

40. A genetically engineered organism that has one or more additional copies of the MAPT-AS1 gene, compared with an equivalent organism that is not genetically engineered to have one or more additional copies of the MAPT-AS1 gene.

41. The genetically engineered organism according to claim 28, wherein the equivalent organism that is not genetically engineered to have additional copies of the MAPT-AS1 gene does not have an endogenous copy of the MAPT-AS1 gene.

42. A method of producing a lncRNA that is capable of modulating the expression of a protein-coding gene, the method comprising;
(a) identifying a population of genes that encode a lncRNA, wherein each member of the population comprises a sequence that overlaps a 5′ untranslated region (UTR), an intron, a coding sequence (CDS), and/or a 3′ UTR of a protein-coding gene, and wherein each member of the population is in antisense orientation with respect to the respective protein-coding gene,
(b) identifying members of the population of genes that encode a lncRNA identified in step (a) that further comprise a MIR domain,
(c) selecting a gene from the population identified in (b), and
(d) causing or allowing a transcript of the selected gene to be expressed, which transcript is the produced lncRNA.

43. The method of claim 42, wherein said modulation is suppression of expression of the respective protein-coding gene that overlaps the gene that encodes the lncRNA if the MIR domain of the lncRNA is in inverse orientation, or wherein said modulation is enhancement of expression of the respective protein-coding gene that overlaps the gene that encodes the lncRNA if the MIR domain of the lncRNA is in direct orientation.

44. The method of claim 42 or claim 43, further comprising the step of isolating the produced lncRNA.

45. A method of selecting a target gene, the method comprising the method of steps (a) and (b) of claim 42, and then
(c) selecting the target gene from a population of protein coding genes that comprises a 5′ untranslated region (UTR), an intron, a coding sequence (CDS), and/or a 3′ UTR that overlaps with a member of the population of genes that encode a lncRNA and comprise a MIR domain, identified in step (b) of claim 42.

46. The method of claim 45, wherein expression of the target gene is identified as being susceptible to being suppressed by a therapeutic RNA if the MIR domain of the overlapping lncRNA gene is in inverse orientation, or wherein the expression of the target gene is identified as being susceptible to being enhanced by a therapeutic RNA if the MIR domain of the overlapping lncRNA gene is in direct orientation.

47. The method of claim 45 or claim 46, wherein any one or more of steps (a)-(c) are be performed in silico.

48. The method of any one of claims 45-47, further comprising the step of providing a therapeutic RNA molecule comprising one or more sequences that correspond with one or more sequences of the overlapping lncRNA.

49. The method of claim 48, wherein the method further comprises a step of modulating the expression of the target gene by contacting a cell that comprises the target gene with the therapeutic RNA.
